(19) United States
(12) Patent Application Publication
(10) Pub. No.: US 2023/0078678 A1
(43) Pub. Date: Mar. 16, 2023

(54) FORMULATIONS OF HUMAN ANTI-TSLP ANTIBODIES AND METHODS OF TREATING ATOPIC DERMATITIS

(71) Applicant: AMGEN INC., Thousand Oaks, CA (US)

(72) Inventors: Alexis Lueras, Camarillo, CA (US); Christopher Sloey, Newbury Park, CA (US)

(21) Appl. No.: 17/760,427

(22) PCT Filed: Feb. 12, 2021
(86) PCT No.: PCT/US21/17880

§ 371 Date: Aug. 9, 2022

Related U.S. Application Data
(60) Provisional application No. 63/148,105, filed on Feb. 10, 2021.
(60) Provisional application No. 62/976,007, filed on Feb. 13, 2020.

(51) Int. Cl. C07K 16/24; A61P 17/00

(52) U.S. Cl. CPC C07K 16/24; A61P 17/00; C07K 2317/94

(57) ABSTRACT

Provided herein are aqueous compositions comprising (a) an anti-TSLP antibody at a concentration greater than about 140 mg/mL, (b) a surfactant, and (c) at least one basic amino acid or a salt thereof. Also provided are aqueous compositions comprising aqueous compositions comprising (a) an anti-TSLP antibody at a concentration greater than about 140 mg/mL, (b) a surfactant, and (c) at least one calcium salt or magnesium salt. Related articles of manufacture, pre-filled syringes, and vials comprising the compositions of the present disclosure are also provided. Uses of the compositions for treating an inflammatory disease, e.g., atopic dermatitis, are provided herein. Also, methods of making a stable, liquid antibody comprising having a viscosity of less than about 100 cP is provided herein.

### CROSS REFERENCE TO RELATED APPLICATIONS

The present application is a national stage of PCT/US2021/17880, filed Feb. 12, 2021 which claims the priority benefit of U.S. Provisional Patent Application No. 62/976,007, filed Feb. 13, 2020, and U.S. Provisional Patent Application No. 63/148,105, filed Feb. 10, 2021, herein incorporated by reference in their entireties.

### FIELD OF THE DISCLOSURE

The disclosure relates to human anti-TSLP monoclonal antibodies, including high-concentration aqueous formulations of tezepelumab and biosimilars thereof.

### INCORPORATION BY REFERENCE OF MATERIAL SUBMITTED ELECTRONICALLY

Incorporated by reference in its entirety is a computer-readable nucleotide/amino acid sequence listing submitted concurrently herewith and identified as follows: 9961 byte ASCII (Text) file named “54250_Seqlisting.txt”; created on Aug. 8, 2022.

### BACKGROUND

### Brief Description of Related Technology

In a recent phase 2, randomized, double-blind, placebo-controlled clinical trial, tezepelumab (also known as AMG 157 and MED9929) was administered to humans at doses ranging from 70 mg to 280 mg. Subjects who received tezepelumab demonstrated lower rates of clinically significant asthma exacerbations than those who received placebo.

Increasing concentrations of protein in drug formulations can cause problems. For example, formulations comprising high concentrations of protein may lead to aggregation resulting in the formation of high molecular weight species (HMWS). HMWS, can be of particular concern in some protein formulations. Aggregation can also potentially affect the subcutaneous bioavailability and pharmacokinetics of a therapeutic protein and also can cause a loss of bioactivity and increase in immunogenicity of the protein. High concentration protein formulations may result in elevated viscosity that can adversely impact drug product filling and administration.

Therefore there is a need in the art for high concentration formulations of tezepelumab with reduced viscosity, high stability and low levels of aggregation.

### SUMMARY

Provided herein for the first time are data demonstrating the viscosity-lowering effects of certain excipients for high concentration antibody formulation. The data support the viscosity-lowering effects of basic amino acids, or a salt thereof, as well as calcium salts or magnesium salts. The data also support the stability of such high concentration antibody formulations.

Accordingly, the present disclosure provides a composition, e.g., aqueous composition, comprising (a) an anti-TSLP antibody at a concentration greater than about 140 mg/mL, (b) a surfactant, and (c) at least one basic amino acid or a salt thereof. Also contemplated is an aqueous composition, comprising (a) an anti-TSLP antibody at a concentration greater than about 140 mg/mL, (b) a surfactant, and (c) at least one basic amino acid or a salt thereof, wherein the composition comprises about 10 mM to about 200 mM basic amino acid or a salt thereof. In exemplary instances, the basic amino acid is arginine. Optionally, the salt is an organic salt of arginine. In various aspects, the arginine salt is arginine acetate, arginine aspartate, arginine glutamate, arginine glycolate, arginine lactate, arginine methanesulfonate, arginine propionate, or a combination thereof. In exemplary instances, the basic amino acid is histidine. Optionally, the salt is an organic salt of histidine. In exemplary aspects, the histidine salt is histidine acetate, histidine aspartate, histidine glutamate, histidine glycolate, histidine lactate, histidine methanesulfonate, histidine propionate, or a combination thereof. In various instances, the basic amino acid is lysine. Optionally the salt is an organic salt of lysine. In various aspects, the lysine salt is lysine acetate, lysine aspartate, lysine glutamate, lysine glycolate, lysine lactate, lysine methanesulfonate, lysine propionate, or a combination thereof.

The present disclosure also provides a composition, e.g., aqueous composition, comprising (a) an anti-TSLP antibody at a concentration greater than about 140 mg/mL, (b) a surfactant, and (c) at least one calcium salt or magnesium salt. Also contemplated is an aqueous composition, comprising (a) an anti-TSLP antibody at a concentration greater than about 140 mg/mL, (b) a surfactant, and (c) at least one calcium salt or magnesium salt, wherein the composition comprises about 15 mM to about 150 mM calcium salt or magnesium salt. In exemplary aspects, the calcium salt or magnesium salt comprises a counterion lacking chloride. Optionally, the counterion is acetate, aspartate, glutamate, glycolate, lactate, methanesulfonate, propionate, or a combination thereof. In various aspects, the calcium salt is calcium acetate, calcium aspartate, calcium glutamate, calcium glycolate, calcium lactate, calcium methanesulfonate, calcium propionate, or a combination thereof. In exemplary instances, the magnesium salt is magnesium acetate, magnesium aspartate, magnesium glutamate, magnesium glycolate, magnesium lactate, magnesium methanesulfonate, magnesium propionate, or a combination thereof. In various instances, the presently disclosed composition comprises about 50 mM to about 150 mM basic amino acid or a salt thereof or about 50 mM to about 150 mM calcium salt or magnesium salt.

In various aspects, the presently disclosed composition further comprises N-acetyl arginine (NAR), N-acetyl lysine, methionine, glycine, proline, sodium acetate, tris acetate, a histidine salt, or a calcium salt, optionally, in an amount of about 50 mM to about 150 mM, or about 50 mM to about 250 mM. In various aspects, the presently disclosed composition comprises (i) an arginine salt and (ii) NAR and/or methionine. In various aspects, the arginine salt is arginine glutamate. In various instances, the presently disclosed composition comprises (i) a calcium salt and (ii) NAR and/or methionine. In exemplary aspects, the calcium salt is calcium glutamate. In exemplary aspects, the presently disclosed composition comprises the anti-TSLP antibody at a concentration of about 160 mg/mL to about 250 mg/mL, optionally, about 160 mg/mL to about 225 mg/mL, e.g., about 170 mg/mL to about 200 mg/mL, optionally, about 175 mg/mL to about 185 mg/mL, e.g., 180 mg/mL. In exemplary instances, the presently disclosed composition has a pH of about 4.5 to about 6.75, optionally, about 4.8 to about 6.0. In exemplary aspects, the viscosity of the presently disclosed composition is less than 100 cP at 23° C., 1000 s⁻¹, optionally, less than 75 cP at 23° C., 1000 s⁻¹, e.g., less than 60 cP or less than 50 cP.

The presently disclosed composition in exemplary instances comprise a surfactant which is amphipathic and/or nonionic. In various aspects, the surfactant is a polysorbate, e.g., polysorbate 20 or polysorbate 80 or a mixture thereof. Optionally, the surfactant is present at a concentration less than or about 0.005% (w/v) to about 0.015% (w/v), optionally, about 0.010% (w/v)±0.0025% (w/v) surfactant, e.g., about 0.005% (w/v), 0.010% (w/v), or 0.015% (w/v) surfactant.

In various embodiments, the aqueous composition comprises 25-190 mM arginine base and 25-200 mM glutamic acid. In various embodiments, the aqueous composition comprises 140 mM arginine base and 150 mM glutamic acid. In various embodiments, the aqueous composition comprising arginine and glutamate comprises proline from 0 to 250 mM. In various embodiments, the aqueous composition comprises 80 mM arginine base, 85 mM glutamic acid and 100 mM L-Proline. In various embodiments, the aqueous composition comprising arginine and glutamate, and optionally proline, comprises 0.01% (w/v) polysorbate 80. In various embodiments, the aqueous composition comprises 140 mM arginine base, 150 mM glutamic acid, 0.01% (w/v) polysorbate 80. In various embodiments, the aqueous composition comprises 80 mM arginine base, 85 mM glutamic acid, 100 mM L-Proline, 0.01% (w/v) polysorbate 80.

In various embodiments, the aqueous composition comprises 10-125 mM arginine base and 25-225 mM glutamic acid. In various embodiments, the aqueous composition comprises 95 mM arginine base and 170 mM glutamic acid. In various embodiments, the aqueous composition comprising arginine and glutamate comprises proline from 0 to 220 mM. In various embodiments, the aqueous composition comprises 50 mM arginine base, 95 mM glutamic acid and 85 mM L-Proline. In various embodiments, the aqueous composition comprising arginine and glutamate comprises 0.01% (w/v) polysorbate 80. In various embodiments, the aqueous composition comprises 95 mM arginine base, 170 mM glutamic acid, 0.01% (w/v) polysorbate 80. In various embodiments, the aqueous composition comprises 50 mM arginine base, 95 mM glutamic acid, 85 mM L-Proline, 0.01% (w/v) polysorbate 80.

In various embodiments, the aqueous composition comprises 15-130 mM calcium and 30-300 mM glutamate. In various embodiments, the aqueous composition comprises 100 mM calcium and 230 mM glutamate. In various embodiments, the aqueous composition comprising calcium and glutamate comprises proline from 0 to 250 mM. In various embodiments, the aqueous composition comprises 60 mM calcium, 140 mM glutamate and 70 mM L-Proline. In various embodiments, the aqueous composition comprises 15-195 mM calcium and 25-320 mM glutamate. In various embodiments, the aqueous composition comprises 110 mM calcium and 240 mM glutamate. In various embodiments, the aqueous composition comprises proline from 0 to 220 mM. In various embodiments, the aqueous composition comprises 70 mM calcium, 145 mM glutamate and 60 mM L-Proline. In various embodiments, the aqueous composition comprising calcium and glutamate comprises 0.01% (w/v) polysorbate 80. In various embodiments, the aqueous composition comprises 100 mM calcium, 230 mM glutamate, 0.01% (w/v) polysorbate 80. In various embodiments, the aqueous composition comprises 60 mM calcium, 140 mM glutamate, 70 mM L-Proline, 0.01% (w/v) polysorbate 80. In various embodiments, the aqueous composition comprises 110 mM calcium, 240 mM glutamate, 0.01% (w/v) Polysorbate 80. In various embodiments, the aqueous composition comprises 70 mM calcium, 145 mM glutamate, 60 mM L-Proline, 0.01% (w/v) polysorbate 80.

In various embodiments, the aqueous composition described herein has a pH of about 4.5 to about 6.75. In various embodiments, the aqueous composition has a pH of about 4.7 to about 6.0. In various embodiments, the aqueous composition has a pH of about 5.1 to about 5.7. In various embodiments, the aqueous composition has a pH of about 4.7 to about 5.3. In various embodiments, the aqueous composition described herein has a pH of about 4.7, 4.8, 4.9, 5.0, 5.1, 5.2, 5.3, 5.4, 5.5, 5.6 or 5.7.

In various instances, the composition is isotonic or has an osmolality in a range of about 200 mOsm/kg to about 500 mOsm/kg, or about 225 mOsm/kg to about 400 mOsm/kg, or about 250 mOsm/kg to about 350 mOsm/kg. Optionally, the composition is isotonic or has an osmolality greater than about 350 mOsm/kg.

In exemplary instances, the composition is suitable for short term storage at 25° C., 30° C., or at 40° C., or long term storage at about −30° C. or about 2° C. to about 8° C. For example, less than 0.5% of the therapeutic protein is degraded after 6 months of storage at 2° C. to 8° C. as determined by Size Exclusion Chromatography (SEC), optionally, wherein the therapeutic protein is contained in glass vials or syringes. In various instances, less than about 5% of the antibody is degraded after storage at about 2° C. to about 8° C. for at least or about 12 months, as determined by Size Exclusion Chromatography (SEC). In various aspects, less than about 5% of the antibody is degraded after storage at about 2° C. to about 8° C. for about 20 months to about 26 months, as determined by Size Exclusion Chromatography (SEC). In exemplary instances, less than about 5% of the antibody is degraded after storage at about 2° C. to about 8° C. for about 30 to about 40 months, as determined by Size Exclusion Chromatography (SEC). In exemplary instances, less than about 5% of the antibody is degraded after storage at about 2° C. to about 8° C. for about 2 years to about 3 years, as determined by Size Exclusion Chromatography (SEC). Also, for example, less than 5% of the antibody is degraded after about 24 months to about 36 months of storage at 2° C. to 8° C. as determined by Size Exclusion Chromatography (SEC), optionally, wherein less than 2% of the antibody is degraded after 24 months or 36 months of storage at 2° C. to 8° C. In various aspects, less than 5% of the antibody is degraded after at least 2 weeks (optionally, after at least 1 month, after at least 2 months, after at least 3 months, after at least 4 months, after at least 5 months or after at least 6 months) of storage at about room temperature (e.g., 25° C.), as determined by SEC. In various instances, less than 5% of the antibody is degraded after about 24 months to about 36 months of storage at 2° C. to 8° C. followed by at least 2 weeks or at least about 1 month or at least about 2 months of storage at about room temperature (e.g., 25° C.), as determined by SEC. Optionally, less than about 5% of the antibody is degraded after storage at a temperature greater than about 20° C. for at least or about 2 weeks, as determined by Size Exclusion Chromatography (SEC), optionally, for at least or about 4 weeks or about 8 weeks. In various aspects, the temperature is greater than or about 25° C. or greater than or about 30° C. or greater than or about 40° C.

An article of manufacture is further provided herein. In exemplary embodiments, the article comprises the composition of the present disclosure, optionally, comprising about 1 mL to about 5 mL (e.g., about 1 mL to about 3 mL) of the aqueous composition.

A pre-filled syringe comprising the presently disclosed composition, optionally, comprising about 1 mL to about 5 mL (e.g., about 1 mL to about 3 mL) of the composition, is additionally provided herein.

Further provided is a vial comprising the presently disclosed composition, optionally, comprising about 1 mL to about 5 mL (e.g., about 1 mL to about 3 mL) of the aqueous composition.

Also provided is an autoinjector containing the aqueous composition described herein. In various embodiments, the auto-injector is an Ypsomed YpsoMate®. In various embodiments, the auto-injector is disclosed in WO 2018/226565, WO 2019/094138, WO 2019/178151, WO 2012/072577, WO2020/081479, WO 2020/081480, PCT/US20/70590, PCT/US20/70591, PCT/US20/53180, PCT/US20/53179, PCT/US20/53178, or PCT/US20/53176.

Use of the presently disclosed composition for treating an inflammatory disease is provided herein. In exemplary aspects, the inflammatory disease is selected from the group consisting of: asthma, atopic dermatitis, chronic obstructive pulmonary disease (COPD), eosinophilic esophagitis (EoE), nasal polyps, chronic spontaneous urticaria, Ig-driven disease (such as IgA nephropathy & lupus nephritis), eosinophilic gastritis, chronic sinusitis without nasal polyps and idiopathic pulmonary fibrosis (IPF). Optionally, the inflammatory disease is atopic dermatitis. Optionally, the inflammatory disease is COPD.

The present disclosure provides a method for treating an inflammatory disease in a subject. In exemplary embodiments, the method comprises administering to the subject a therapeutically effective amount of the presently disclosed composition. In various aspects, the inflammatory disease is selected from the group consisting of: asthma, atopic dermatitis, chronic obstructive pulmonary disease (COPD), eosinophilic esophagitis (EoE), nasal polyps, chronic spontaneous urticarial, Ig-driven disease (such as IgA nephropathy & lupus nephritis), eosinophilic gastritis, chronic sinusitis without nasal polyps and idiopathic pulmonary fibrosis (IPF). Optionally, the inflammatory disease is atopic dermatitis. Optionally, the inflammatory disease is COPD. In various instances, the presently disclosed composition is administered to the subject by subcutaneous administration. In exemplary instances, about 1 mL to about 5 mL (e.g., about 1 mL to about 3 mL) of the aqueous composition is administered to the subject.

Further provided is a method of making a stable, liquid antibody composition having a viscosity of less than about 100 cP and comprising (A) an anti-TSLP antibody at a concentration greater than about 140 mg/mL, (B) a surfactant, and (C) a basic amino acid or a salt thereof, a calcium salt, a magnesium salt, or a combination thereof. In exemplary embodiments, the method comprises: (i) combining the antibody with an aqueous solution comprising about 50 mM to about 150 mM basic amino acid or a salt thereof, a calcium salt, a magnesium salt, or a combination thereof and (ii) adding a surfactant to achieve a final concentration of about 0.01% (w/v)±0.005% (w/v) surfactant.

Further aspects and advantages will be apparent to those of ordinary skill in the art from a review of the following detailed description, taken in conjunction with the drawings. While the compositions, articles, and methods are susceptible of embodiments in various forms, the description hereafter includes specific embodiments with the understanding that the disclosure is illustrative, and is not intended to limit the invention to the specific embodiments described herein. For the compositions, articles, and methods described herein, optional features, including but not limited to components, compositional ranges thereof, substituents, conditions, and steps, are contemplated to be selected from the various aspects, embodiments, and examples provided herein.

### BRIEF DESCRIPTION OF THE DRAWINGS

FIG. 1 is a graph of the viscosity (cP) of two different formulations comprising a therapeutic protein plotted as a function of protein concentration (mg/mL).

FIG. 2 is a graph of the viscosity (cP) of several different tezepelumab formulations comprising the indicated excipient (at the indicated amount of 100 mM or 150 mM). The viscosity of a control comprising no excipient is also provided. Each formulation comprised tezepelumab at a concentration of about 210 mg/mL.

FIG. 3 is a graph of the viscosity (cP) of several different tezepelumab formulations comprising the indicated excipient (at the indicated amount, 60 mM). The viscosity of a control comprising no excipient is also provided. Each formulation comprised tezepelumab at a concentration of about 190 mg/mL.

FIG. 4 is a graph of the viscosity (cP) of several different tezepelumab formulations comprising the indicated excipient(s) (at the indicated amount). The viscosity of a control comprising no excipient is also provided. Each formulation comprised tezepelumab at a concentration of about 210 mg/mL. The bar immediately left of 0.5% PVP is for the formulation comprising 100 mM sodium acetate and 75 mM arginine acetate. The % noted are % (w/v).

FIG. 5 is a graph of the viscosity (cP) of several different tezepelumab formulations comprising the indicated excipient(s) (at the indicated amount (mM)). The viscosity of a control comprising no excipient is also provided. Each formulation comprised tezepelumab at a concentration of about 210 mg/mL.

FIG. 6 is a graph of the viscosity (cP) of several different tezepelumab formulations comprising the indicated excipient (at the indicated amount (60 mM)). The viscosity of a control comprising no excipient is also provided. Each formulation comprised tezepelumab at a concentration of about 190 mg/mL.

FIG. 7 is a graph of the viscosity (cP) of two different tezepelumab formulations comprising the indicated excipient (at the indicated amount (60 mM)). The viscosity of a control comprising no excipient is also provided. Each formulation comprised tezepelumab at a concentration of about 190 mg/mL.

FIG. 8 is a table of the protein concentration, viscosity and pH of several different tezepelumab formulations comprising the indicated excipient(s) (at the indicated amount (mM)).

FIG. 9A is a graph of the viscosity (cP) of several different tezepelumab formulations comprising the indicated excipient (at the indicated amount (150 mM)). The viscosity of a control comprising no excipient is also provided. Each formulation comprised tezepelumab at a concentration of about 210 mg/mL.

FIG. 9B is a graph of the viscosity (cP) of several different tezepelumab formulations comprising the indicated excipient (at the indicated amount (90 mM)). The viscosity of a control comprising no excipient is also provided. Each formulation comprised tezepelumab at a concentration of about 195 mg/mL.

FIG. 10 is a graph of the viscosity (cP) of several different tezepelumab formulations comprising the indicated excipient (at the indicated amount (mM)). The viscosity of a control comprising no excipient is also provided. Each formulation comprised tezepelumab at a concentration of about 210 mg/mL.

FIG. 11 is a graph of the viscosity (cP) of several different tezepelumab formulations comprising the indicated excipient (at the indicated amount (150 mM)). The viscosity of a control comprising no excipient is also provided. Each formulation comprised tezepelumab at a concentration of about 210 mg/mL.

FIG. 12 is a graph of the viscosity (cP) of several different tezepelumab formulations comprising the indicated excipient (at the indicated amount (50 mM-150 mM)). The viscosity of a control comprising no excipient is also provided. Each formulation comprised tezepelumab at a concentration of about 210 mg/mL.

FIG. 13A is a graph of the viscosity (cP) of several different tezepelumab formulations comprising 150 mM arginine acetate (at the indicated pH (4.75-5.7)). The viscosities of a control comprising no excipient and a formulation comprising 150 mM proline are also provided. Each formulation comprised tezepelumab at a concentration of about 210 mg/mL

FIG. 13B is a graph of the viscosity (cP) of several different tezepelumab formulations comprising 60 mM Histidine acetate (at the indicated pH (5.5-6.5)). Each formulation comprised tezepelumab at a concentration of about 210 mg/mL.

FIG. 14 is a graph of the viscosity (cP) of several different tezepelumab formulations comprising the indicated excipient(s) (at the indicated amount (33 mM-150 mM)). The viscosity of a control comprising no excipient is also provided. Each formulation comprised tezepelumab at a concentration of about 210 mg/mL.

FIGS. 15A-15D show size exclusion chromatography (SEC) analysis of different anti-TSLP formulations during stress conditions: FIG. 15A, −30° C.; FIG. 15B, 5° C., FIG. 15C, 25° C., FIG. 15D, 40° C.

FIG. 16 shows cation exchange chromatography (CEX) analysis (Main Peak %) of different anti-TSLP formulations during stress conditions.

FIG. 17 shows RCE-SDS analysis of heavy chain and light chain release and stability at various storage conditions over 6 months.

FIG. 18 shows viscosity of different anti-TSLP formulations during stress conditions.

### DETAILED DESCRIPTION

### Definitions

The foregoing description is given for clearness of understanding only, and no unnecessary limitations should be understood therefrom, as modifications within the scope of the invention may be apparent to those having ordinary skill in the art.

Throughout this specification and the claims which follow, unless the context requires otherwise, the word “comprise” and variations such as “comprises” and “comprising” will be understood to imply the inclusion of a stated integer or step or group of integers or steps but not the exclusion of any other integer or step or group of integers or steps.

Throughout the specification, where compositions are described as including components or materials, it is contemplated that the compositions can also consist essentially of, or consist of, any combination of the recited components or materials, unless described otherwise. Likewise, where methods are described as including particular steps, it is contemplated that the methods can also consist essentially of, or consist of, any combination of the recited steps, unless described otherwise. The invention illustratively disclosed herein suitably may be practiced in the absence of any element or step which is not specifically disclosed herein.

The practice of a method disclosed herein, and individual steps thereof, can be performed manually and/or with the aid of or automation provided by electronic equipment. Although processes have been described with reference to particular embodiments, a person of ordinary skill in the art will readily appreciate that other ways of performing the acts associated with the methods may be used. For example, the order of various of the steps may be changed without departing from the scope or spirit of the method, unless described otherwise. In addition, some of the individual steps can be combined, omitted, or further subdivided into additional steps.

The compositions and methods are contemplated to include embodiments including any combination of one or more of the additional optional elements, features, and steps further described below (including those shown in the figures), unless stated otherwise.

In jurisdictions that forbid the patenting of methods that are practiced on the human body, the meaning of “administering” of a composition to a human subject shall be restricted to prescribing a controlled substance that a human subject will self-administer by any technique (e.g., orally, inhalation, topical application, injection, insertion, etc.). The broadest reasonable interpretation that is consistent with laws or regulations defining patentable subject matter is intended. In jurisdictions that do not forbid the patenting of methods that are practiced on the human body, the “administering” of compositions includes both methods practiced on the human body and also the foregoing activities.

It should be understood that every maximum numerical limitation given throughout this specification includes as alternative aspects ranges formed with every corresponding lower numerical limitation, as if such ranges were expressly written. Every minimum numerical limitation given throughout this specification will include as alternative aspects ranges formed with every higher numerical limitation, as if such ranges were expressly written. Every numerical range given throughout this specification will include every narrower numerical range that falls within such broader numerical range, as if such narrower numerical ranges were all expressly written herein. The dimensions and values disclosed herein should be understood to include disclosure of both the recited value and the corresponding exact numerical, e.g., a value described as “about 10 mM” should be understood to include, as an alternative disclosure, “10 mM.”

All patents, publications and references cited herein are hereby fully incorporated by reference. In case of conflict between the present disclosure and incorporated patents, publications and references, the present disclosure should control.

Unless otherwise stated, the following terms used in this application, including the specification and claims, have the definitions given below.

As used in the specification and the appended claims, the indefinite articles “a” and “an” and the definite article “the” include plural as well as singular referents unless the context clearly dictates otherwise.

Unless otherwise defined, all technical and scientific terms used herein have the same meaning as commonly understood by one of ordinary skill in the art to which the present disclosure belongs. The following references provide one of skill with a general definition of many of the terms used in this disclosure include, but are not limited to: Singleton et al., DICTIONARY OF MICROBIOLOGY AND MOLECULAR BIOLOGY (2d Ed. 1994); THE CAMBRIDGE DICTIONARY OF SCIENCE AND TECHNOLOGY (Walker Ed., 1988); THE GLOSSARY OF GENETICS, 5th Ed., R. Rieger et al. (Eds.), Springer Verlag (1991); and Hale & Marham, THE HARPER COLLINS DICTIONARY OF BIOLOGY (1991).

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the term “about” or “approximately” means within 30%, 25%, 20%, 15%, 10%, 9%, 8%, 7%, 6%, 5%, 4%, 3%, 2%, 1%, 0.5%, or 0.05% of a given value or range. Whenever the term “about” or “approximately” precedes the first numerical value in a series of two or more numerical values, it is understood that the term “about” or “approximately” applies to each one of the numerical values in that series.

The term “specifically binds” is “antigen specific”, is “specific for”, “selective binding agent”, “specific binding agent”, “antigen target” or is “immunoreactive” with an antigen refers to an antibody or polypeptide that binds an target antigen with greater affinity than other antigens of related proteins. It is contemplated herein that the agent specifically binds target proteins, for example, a surface antigen (e.g., T cell receptor, CD3), a cytokine (e.g., TSLP, IL-4, IL-5, IL-13, IL-17, IFN-g, TNF-a) and the like.

The term “antibody” or “immunoglobulin” refers to the canonical tetrameric glycoprotein that consists of two substantially full-length heavy chains and two substantially full-length light chains, each comprising a variable region and a substantially full-length constant region. Antigen-binding portions may be produced by recombinant DNA techniques or by enzymatic or chemical cleavage of intact antibodies. The term “antibody” includes monoclonal antibodies, polyclonal antibodies, chimeric antibodies, human antibodies, and humanized antibodies.

Antibody variants include antibody fragments and antibody like proteins with changes to structure of canonical tetrameric antibodies. Typically antibody variants include V regions with a change to the constant regions, or, alternatively, adding V regions to constant regions, optionally in a non-canonical way. Examples include multispecific antibodies (e.g., bispecific antibodies with extra V regions), antibody fragments that can bind an antigen (e.g., Fab′, F′(ab)2, Fv, single chain antibodies, diabodies), biparatopic and recombinant peptides comprising the forgoing as long as they exhibit the desired biological activity.

Antibody fragments include antigen-binding portions of the antibody including, inter alia, Fab, Fab′, F(ab′)2, Fv, domain antibody (dAb), complementarity determining region (CDR) fragments, CDR-grafted antibodies, single-chain antibodies (scFv), single chain antibody fragments, chimeric antibodies, diabodies, triabodies, tetrabodies, minibody, linear antibody; chelating recombinant antibody, a tribody or bibody, an intrabody, a nanobody, a small modular immunopharmaceutical (SMIP), an antigen-binding-domain immunoglobulin fusion protein, single domain antibodies (including camelized antibody), a VHH containing antibody, or a variant or a derivative thereof, and polypeptides that contain at least a portion of an immunoglobulin that is sufficient to confer specific antigen binding to the polypeptide, such as one, two, three, four, five or six CDR sequences, as long as the antibody retains the desired biological activity.

“Valency” refers to the number of antigen binding sites on each antibody or antibody fragment that targets an epitope. A typical full length IgG molecule, or F(ab)2 is “bivalent” in that it has two identical target binding sites. A “monovalent’ antibody fragment such as a F(ab)′ or scFc with a single antigen binding site. Trivalent or tetravalent antigen binding proteins can also be engineered to be multivalent.

“Monoclonal antibody” refers to an antibody obtained from a population of substantially homogeneous antibodies, i.e., the individual antibodies comprising the population are identical except for possible naturally occurring mutations that may be present in minor amounts.

The term “inhibits TSLP activity” includes inhibiting any one or more of the following:—
binding of TSLP to its receptor;
proliferation, activation, or differentiation of cells expressing TSLPR in the presence of TSLP;
inhibition of Th2 cytokine production in a polarization assay in the presence of TSLP;
dendritic cell activation or maturation in the presence of TSLP;
mast cell cytokine release in the presence of TSLP.
   See, e.g., U.S. Pat. No. 7,982,016 B2, column 6 and example 8 and US 2012/0020988 A1, Examples 7-10.

The term “sample” or “biological sample” refers to a specimen obtained from a subject for use in the present methods, and includes urine, whole blood, plasma, serum, saliva, sputum, tissue biopsies, cerebrospinal fluid, peripheral blood mononuclear cells with in vitro stimulation, peripheral blood mononuclear cells without in vitro stimulation, gut lymphoid tissues with in vitro stimulation, gut lymphoid tissues without in vitro stimulation, gut lavage, bronchioalveolar lavage, nasal lavage, and induced sputum.

The terms “treat”, “treating” and “treatment” refer to eliminating, reducing, suppressing or ameliorating, either temporarily or permanently, either partially or completely, a clinical symptom, manifestation or progression of an event, disease or condition associated with an inflammatory disorder described herein. As is recognized in the pertinent field, drugs employed as therapeutic agents may reduce the severity of a given disease state, but need not abolish every manifestation of the disease to be regarded as useful therapeutic agents. Similarly, a prophylactically administered treatment need not be completely effective in preventing the onset of a condition in order to constitute a viable prophylactic agent. Simply reducing the impact of a disease (for example, by reducing the number or severity of its symptoms, or by increasing the effectiveness of another treatment, or by producing another beneficial effect), or reducing the likelihood that the disease will occur or worsen in a subject, is sufficient. One embodiment of the disclosure is directed to a method for determining the efficacy of treatment comprising administering to a patient therapeutic agent in an amount and for a time sufficient to induce a sustained improvement over baseline of an indicator that reflects the severity of the particular disorder.

The term “therapeutically effective amount” refers to an amount of therapeutic agent that is effective to ameliorate or lessen symptoms or signs of disease associated with a disease or disorder.

The term “cytokine” as used herein refers to one or more small (5-20 kD) proteins released by cells that have a specific effect on interactions and communications between cells or on the behavior of cells, such as immune cell proliferation and differentiation. Functions of cytokines in the immune system include, promoting influx of circulating leukocytes and lymphocytes into the site of immunological encounter; stimulating the development and proliferation of B cells, T cells, peripheral blood mononuclear cells (PBMCs) and other immune cells; and providing antimicrobial activity. Exemplary immune cytokines, include but are not limited to, IL-1, IL-2, IL-3, IL-4, IL-5, IL-6, IL-7, IL-9, IL-10, IL-12, IL-13, IL-15, IL17A, IL-17F, IL-18, IL-21, IL-22, interferon (including IFN alpha, beta, and gamma), tumor necrosis factor (including TNF alpha, beta), transforming growth factor (including TGF alpha, beta), granulocyte colony stimulating factor (GCSF), granulocyte macrophage colony stimulating factor (GMCSF) and thymic stromal lymphopoietin (TSLP).

A “T helper (Th) 1 cytokine” or “Th1-specific cytokine” refers to cytokines that are expressed (intracellularly and/or secreted) by Th1 T cells, and include IFN-g, TNF-α, IL-12. A “Th2 cytokine” or “Th2-specific cytokine” refers to cytokines that are expressed (intracellularly and/or secreted) by Th2 T cells, including IL-4, IL-5, IL-13, and IL-10. A “Th17 cytokine” or “Th17-specific cytokine” refers to cytokines that are expressed (intracellularly and/or secreted) by Th17 T cells, including IL-17A, IL-17F, IL-22 and IL-21. Certain populations of Th17 cells express IFN-g and/or IL-2 in addition to the Th17 cytokines listed herein. A polyfunctional CTL cytokine includes IFN-g, TNF-α, IL-2 and IL-17.

Low-Viscosity Anti-TSLP Antibody Compositions

Tezepelumab has shown effectiveness at strengths ranging from 70 mg to 280 mg and the anti-TSLP antibody, in some instances, will be formulated at doses of 110 mg/mL or 140 mg/mL. Formulations with high protein concentrations may exhibit increased viscosity to a point where the functionality of the device used to administer the antibody to the patient may be negatively impacted. Similarly, the ability of a health care provider to manually inject the drug into the patient may be compromised. High viscosity can additionally be prohibitive during manufacturing. Formulations with high protein concentrations also are challenging from the standpoint of protein stability. For example, aggregation resulting in the formation of high molecular weight species (HMWS) can occur in formulations comprising high concentrations of protein. It is therefore desirable to provide a low viscosity, isotonic, liquid formulation of an anti-TSLP antibody, such as tezepelumab, suitable for parenteral administration that can be stored long term at cold temperatures (e.g., at 2-8° C. and −30° C.) or short term at room temperature (e.g., 20-25° C., for patient convenience).

To overcome the high viscosity issue of formulations with high protein concentrations, the present disclosure provides a composition, e.g., aqueous composition, comprising (a) an anti-TSLP antibody at a concentration greater than about 140 mg/mL, (b) a surfactant, and (c) at least one basic amino acid or a salt thereof. The present disclosure also provides a composition, e.g., aqueous composition, comprising (a) an anti-TSLP antibody at a concentration greater than about 140 mg/mL, (b) a surfactant, and (c) at least one calcium salt or magnesium salt. Based at least in part on data provided herein, and without being bound to any particular theory, the presently disclosed compositions represent low viscosity compositions comprising a high concentration of the therapeutic protein which may be administered to a patient in need thereof without any complications due to high viscosity. Based at least in part on data provided herein, and without being bound to any particular theory, the presently disclosed compositions are highly stable inasmuch as the presently disclosed compositions exhibit minimal degradation after storage (short and/or long-term storage) at cold temperatures (e.g., at 2-8° C. and −30° C.) or short term at room temperature (e.g., approximately 20-25° C.).

Basic Amino Acids

In exemplary embodiments, the presently disclosed composition comprises a basic amino acid, or a salt thereof. As used herein, the term “basic amino acid” refers to an amino acid having a basic side chain at neutral pH. The pKa of a basic amino acid is high enough that they tend to bind protons, gaining a positive charge in the process. The basic amino acid in exemplary aspects comprises a side chain comprising a nitrogen which bind to protons (and become protonated) or release binding to a proton (and become deprotonated). In exemplary aspects, the basic amino acid may equilibrate between NH2 (deprotonated) and NH3+ (protonated) forms or between NH (deprotonated) and NH2+ (protonated) forms or between N (deprotonated) and NH+ (protonated) forms. For a basic amino acid at physiological pH, e.g., about pH 7.0, the protonated forms dominate. In exemplary aspects, the basic amino acid is arginine (Arg; R) or lysine (Lys, K) or histidine (His, H). While the basic amino acid may be either the D-isomer of the L-isomer, in exemplary instances, the basic amino acid is the L-isomer of the amino acid, e.g., L-Arg, L-Lys, L-His. In exemplary instances, the basic amino acid is arginine. In exemplary instances, the basic amino acid is histidine. In various instances, the basic amino acid is lysine.

In exemplary aspects, the basic amino acid is a derivative of arginine, e.g., L-2-amino-3-guanidinopropionic acid, 4-guanidinobutyric acid. In exemplary aspects, the basic amino acid comprises a structure of Formula I:

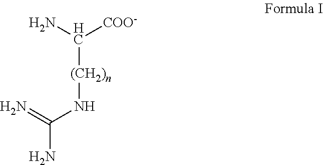

wherein n is 1 to 16, or 1 to 10, or 1 to 7, or 1 to 6, or 2 to 6, or 2 or 3 or 4 or 5.

In exemplary aspects, the basic amino acid is a derivative of lysine, e.g., 5-hydoxylysine, ornithine, N-acetyl-L-lysine, 2,4-diaminobutyric acid.

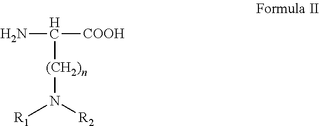

wherein n is 1 to 16, or 1 to 10, or 1 to 7, or 1 to 6, or 2 to 6, or 2 or 3 or 4 or 5, each of R₁ and R₂ is independently selected from the group consisting of H, C₁-C₁₈ alkyl, (C₁-C₁₈ alkyl)OH, (C₁-C₁₈ alkyl)NH₂, (C₁-C₁₈ alkyl)SH, (C₀-C₄ alkyl)(C₃-C₆)cycloalkyl, (C₀-C₄ alkyl)(C₂-C₆ heterocyclic), (C₀-C₄ alkyl)(C₆-C₁₀ aryl)R₇, and (C₁-C₄ alkyl)(C₃-C₉ heteroaryl), wherein R₇ is H or OH.

In exemplary aspects, the basic amino acid is a derivative of histidine, e.g., desaminohistidine, hydroxyl-histidine, acetyl-histidine, homo-histidine, N-methyl histidine, alpha-methyl histidine, imidazole acetic acid, or alpha, alpha-dimethyl imidiazole acetic acid (DMIA).

In various aspects, the presently disclosed composition comprises a salt of a basic amino acid. In exemplary instances, the salt is a pharmaceutically acceptable salt. As used herein the term “pharmaceutically acceptable salt” refers to salts of compounds that retain the biological activity of the parent compound, and which are not biologically or otherwise undesirable. Such salts can be prepared in situ during the final isolation and purification of the analog, or separately prepared by reacting a free base function with a suitable acid. Many of the compounds disclosed herein are capable of forming acid and/or base salts by virtue of the presence of amino and/or carboxyl groups or groups similar thereto.

Pharmaceutically acceptable acid addition salts may be prepared from inorganic and organic acids. Representative acid addition salts include, but are not limited to acetate, adipate, alginate, citrate, aspartate, benzoate, benzenesulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate, and undecanoate. Salts derived from inorganic acids include hydrochloric acid, hydrobromic acid, sulfuric acid, nitric acid, phosphoric acid, and the like. Salts derived from organic acids include acetic acid, propionic acid, glycolic acid, pyruvic acid, oxalic acid, malic acid, malonic acid, succinic acid, maleic acid, fumaric acid, tartaric acid, citric acid, benzoic acid, cinnamic acid, mandelic acid, methanesulfonic acid, ethanesulfonic acid, p-toluene-sulfonic acid, salicylic acid, and the like. Examples of acids which can be employed to form pharmaceutically acceptable acid addition salts include, for example, an inorganic acid, e.g., hydrochloric acid, hydrobromic acid, sulphuric acid, and phosphoric acid, and an organic acid, e.g., oxalic acid, maleic acid, succinic acid, and citric acid.

Basic addition salts also can be prepared in situ during the final isolation and purification of the source of salicylic acid, or by reacting a carboxylic acid-containing moiety with a suitable base such as the hydroxide, carbonate, or bicarbonate of a pharmaceutically acceptable metal cation or with ammonia or an organic primary, secondary, or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylammonium, dimethylammonium, trimethylammonium, triethylammonium, diethylammonium, and ethylammonium, amongst others. Other representative organic amines useful for the formation of base addition salts include, for example, ethylenediamine, ethanolamine, diethanolamine, piperidine, piperazine, and the like. Salts derived from organic bases include, but are not limited to, salts of primary, secondary and tertiary amines.

Further, basic nitrogen-containing groups can be quaternized with the analog of the present disclosure as lower alkyl halides such as methyl, ethyl, propyl, and butyl chlorides, bromides, and iodides; long chain halides such as decyl, lauryl, myristyl, and stearyl chlorides, bromides, and iodides; arylalkyl halides like benzyl and phenethyl bromides and others. Water or oil-soluble or dispersible products are thereby obtained.

In exemplary aspects, the basic amino acid is arginine and the presently disclosed composition comprises an arginine salt. In various aspects, the arginine salt is an organic salt of arginine. In various aspects, the arginine salt is arginine acetate, arginine aspartate, arginine glutamate, arginine glycolate, arginine lactate, arginine methanesulfonate, arginine propionate, or a combination thereof.

In exemplary aspects, the basic amino acid is histidine and the presently disclosed composition comprises a histidine salt. In various aspects, the histidine salt is an organic salt of histidine. In exemplary aspects, the histidine salt is histidine acetate, histidine aspartate, histidine glutamate, histidine glycolate, histidine lactate, histidine methanesulfonate, histidine propionate, or a combination thereof.

In exemplary aspects, the basic amino acid is lysine and the presently disclosed composition comprises a lysine salt. In various aspects, the lysine salt is an organic salt of lysine. In various aspects, the lysine salt is lysine acetate, lysine aspartate, lysine glutamate, lysine glycolate, lysine lactate, lysine methanesulfonate, lysine propionate, or a combination thereof.

In various instances, the presently disclosed composition comprises about 10 mM to about 300 mM, or about 50 mM to about 300 mM basic amino acid or a salt thereof. In exemplary aspects, the presently disclosed composition comprises about 10 mM to about 200 mM, about 50 mM to about 250 mM, about 50 mM to about 200 mM, about 50 mM to about 150 mM, about 50 mM to about 100 mM, about 50 mM to about 90 mM, about 50 mM to about 80 mM, about 50 mM to about 70 mM, about 50 mM to about 60 mM, about 50 mM to about 55 mM, about 55 mM to about 200 mM, about 60 mM to about 200 mM, about 70 mM to about 200 mM, about 80 mM to about 200 mM, about 90 mM to about 200 mM, about 100 mM to about 200 mM, about 150 mM to about 200 mM, about 160 mM to about 200 mM, about 170 mM to about 200 mM, about 180 mM to about 200 mM, or about 190 mM to about 200 mM basic amino acid or a salt thereof. In various instances, the presently disclosed composition comprises about 50 mM to about 100 mM (e.g., about 50 mM, about 60 mM, about 70 mM, about 80 mM, about 90 mM, about 100 mM) or about 100 mM to about 200 mM (e.g., about 100 mM, about 110 mM, about 120 mM, about 130 mM, about 140 mM, about 150 mM, about 160 mM, about 170 mM, about 180 mM, about 190 mM, about 200 mM) basic amino acid or a salt thereof. In various instances, the presently disclosed composition comprises about 50 mM to about 100 mM or about 50 mM to about 75 mM or about 75 mM to about 100 mM basic amino acid or a salt thereof. In various instances, the presently disclosed composition comprises about 100 mM to about 200 mM or about 100 mM to about 150 mM or about 150 mM to about 200 mM basic amino acid or a salt thereof. In exemplary aspects, the presently disclosed composition comprises about 10 mM to about 200 mM basic amino acid or salt thereof.

In various instances, the anti-TSLP antibody, e.g., tezepelumab, is formulated with about 25 mM to about 190 mM arginine and about 25 mM to about 200 mM glutamate. In various aspects, the anti-TSLP antibody, e.g., tezepelumab, is formulated with 100 mM to about 180 mM arginine (e.g., about 100 mM to about 170 mM, about 100 mM to about 160 mM, about 100 mM to about 150 mM, about 100 mM to about 140 mM, about 100 mM to about 130 mM, about 100 mM to about 120 mM, about 100 mM to about 110 mM, about 110 mM to about 180 mM, about 120 mM to about 180 mM, about 130 mM to about 180 mM, about 140 mM to about 180 mM, about 150 mM to about 180 mM, about 160 mM to about 180 mM, about 170 mM to about 180 mM, about 120 mM to about 170 mM, about 130 mM to about 160 mM, about 135 mM to about 155 mM) and about 110 mM to about 240 mM glutamate (e.g., about 110 mM to about 180 mM, about 110 mM to about 170 mM, about 110 mM to about 160 mM, about 110 mM to about 150 mM, about 110 mM to about 140 mM, about 110 mM to about 130 mM, about 110 mM to about 120 mM, about 120 mM to about 180 mM, about 130 mM to about 180 mM, about 140 mM to about 180 mM, about 150 mM to about 180 mM, about 160 mM to about 180 mM, about 170 mM to about 180 mM, about 120 mM to about 170 mM, about 130 mM to about 160 mM, about 140 mM to about 160 mM, about 145 mM to about 155 mM glutamate). In various instances, the anti-TSLP antibody, e.g., tezepelumab, is formulated with about 135 mM to about 145 mM arginine and about 145 mM to about 155 mM glutamate. In various instances, the anti-TSLP antibody, e.g., tezepelumab, is formulated in about 10 mM to about 125 mM arginine and about 25 mM to about 225 mM glutamate. In various aspects, the anti-TSLP antibody, e.g., tezepelumab, is formulated in about 55 mM to about 135 mM arginine (e.g., about 55 mM to about 125 mM, about 55 mM to about 115 mM, about 55 mM to about 105 mM, about 55 mM to about 95 mM, about 55 mM to about 85 mM, about 55 mM to about 75 mM, about 55 mM to about 65 mM, about 65 mM to about 135 mM, about 75 mM to about 135 mM, about 85 mM to about 135 mM, about 95 mM to about 135 mM, about 105 mM to about 135 mM, about 115 mM to about 145 mM, about 125 mM to about 135 mM, about 75 mM to about 115 mM, about 85 mM to about 105 mM arginine) and about 130 mM to about 210 mM glutamate (e.g., about 130 mM to about 200 mM, about 130 mM to about 240 mM, about 130 mM to about 180 mM, about 130 mM to about 170 mM, about 130 mM to about 160 mM, about 130 mM to about 150 mM, about 130 mM to about 140 mM, about 140 mM to about 210 mM, about 150 mM to about 210 mM, about 160 mM to about 210 mM, about 170 mM to about 210 mM, about 180 mM to about 210 mM, about 190 mM to about 210 mM, about 200 mM to about 210 mM, about 150 mM to about 190 mM, about 160 mM to about 180 mM, about 165 mM to about 175 mM glutamate). In exemplary embodiments, the presently disclosed composition comprises greater than 140 mg/mL tezepelumab, about 85.5 mM to about 104.5 mM arginine, 153 mM to about 187 mM glutamate, and 0.01% (w/v) polysorbate 80. In exemplary embodiments, the presently disclosed composition comprises greater than 140 mg/mL tezepelumab, about 95 mM arginine, 170 mM glutamate, and 0.01% (w/v) polysorbate 80. Optionally, the pH is about 5.4±0.2, or about 5.4±0.1.

Calcium Salts and Magnesium Salts

In exemplary embodiments, the presently disclosed composition comprises a calcium salt or magnesium salt. In various aspects, the calcium salt or the magnesium salt comprises any counterion. In exemplary aspects, the calcium salt or magnesium salt comprises a counterion lacking chloride. In exemplary instances, the counterion is acetate, aspartate, glutamate, glycolate, lactate, methanesulfonate, propionate, or a combination thereof. In various aspects, the calcium salt is calcium acetate, calcium aspartate, calcium glutamate, calcium glycolate, calcium lactate, calcium methanesulfonate, calcium propionate, or a combination thereof. In exemplary instances, the magnesium salt is magnesium acetate, magnesium aspartate, magnesium glutamate, magnesium glycolate, magnesium lactate, magnesium methanesulfonate, magnesium propionate, or a combination thereof.

In various instances, the presently disclosed composition comprises about 15 mM to about 300 mM, about 15 mM to about 200 mM, or about 50 mM to about 150 mM calcium salt or magnesium salt. In various instances, the presently disclosed composition comprises about 15 mM to about 300 mM, or about 50 mM to about 300 mM calcium salt or magnesium salt. In exemplary aspects, the presently disclosed composition comprises about 15 mM to about 200 mM, about 15 to about 150 mM, about 50 mM to about 250 mM, about 50 mM to about 200 mM, about 50 mM to about 150 mM, about 50 mM to about 100 mM, about 50 mM to about 90 mM, about 50 mM to about 80 mM, about 50 mM to about 70 mM, about 50 mM to about 60 mM, about 50 mM to about 55 mM, about 55 mM to about 200 mM, about 60 mM to about 200 mM, about 70 mM to about 200 mM, about 80 mM to about 200 mM, about 90 mM to about 200 mM, about 100 mM to about 200 mM, about 150 mM to about 200 mM, about 160 mM to about 200 mM, about 170 mM to about 200 mM, about 180 mM to about 200 mM, or about 190 mM to about 200 mM calcium salt or magnesium salt. In various instances, the presently disclosed composition comprises about 50 mM to about 100 mM (e.g., about 50 mM, about 60 mM, about 70 mM, about 80 mM, about 90 mM, about 100 mM) or about 100 mM to about 200 mM (e.g., about 100 mM, about 110 mM, about 120 mM, about 130 mM, about 140 mM, about 150 mM, about 160 mM, about 170 mM, about 180 mM, about 190 mM, about 200 mM) calcium salt or magnesium salt. In various instances, the presently disclosed composition comprises about 15 mM to about 130 mM, about 50 mM to about 100 mM or about 50 mM to about 75 mM or about 75 mM to about 100 mM calcium salt or magnesium salt. In various instances, the presently disclosed composition comprises about 100 mM to about 200 mM or about 100 mM to about 150 mM or about 150 mM to about 200 mM calcium salt or magnesium salt.

In various instances, the anti-TSLP antibody, e.g., tezepelumab, is formulated with about 15 mM to about 130 mM calcium and about 30 mM to about 300 mM glutamate. In various aspects, the anti-TSLP antibody, e.g., tezepelumab, is formulated with about 70 mM to about 130 mM calcium (e.g., about 80 mM to about 130 mM, about 90 mM to about 130 mM, about 100 mM to about 130 mM, about 110 mM to about 130 mM, about 120 mM to about 130 mM, about 70 mM to about 120 mM, about 70 mM to about 110 mM, about 70 mM to about 100 mM, about 70 mM to about 90 mM, about 70 mM to about 80 mM, about 80 mM to about 110 mM, about 90 mM to about 110 mM, about 95 mM to about 105 mM calcium) and about 190 mM to about 270 mM glutamate (e.g., about 190 mM to about 260 mM, about 190 mM to about 250 mM, about 190 mM to about 240 mM, about 190 mM to about 230 mM, about 190 mM to about 220 mM, about 190 mM to about 210 mM, about 190 mM to about 200 mM, about 200 mM to about 270 mM, about 210 mM to about 270 mM, about 220 mM to about 270 mM, about 230 mM to about 270 mM, about 240 mM to about 270 mM, about 250 mM to about 270 mM, about 260 mM to about 270 mM, about 210 mM to about 250 mM, about 220 mM to about 240 mM, about 230 mM to about 240 mM, about 225 mM to about 235 mM, or about 235 to 245 mM glutamate). In exemplary aspects, the anti-TSLP antibody, e.g., tezepelumab, is formulated with about 95 mM to about 105 mM calcium and about 225 mM to about 235 mM glutamate, or about 235 to 245 mM glutamate. In various instances, the anti-TSLP antibody, e.g., tezepelumab, is formulated in about 15 mM to about 195 mM calcium and about 25 mM to about 320 mM glutamate. In various aspects, the anti-TSLP antibody, e.g., tezepelumab, is formulated in about 70 mM to about 150 mM calcium (e.g., about 80 mM to about 150 mM, about 90 mM to about 150 mM, about 100 mM to about 150 mM, about 110 mM to about 150 mM, about 120 mM to about 150 mM, about 130 mM to about 150 mM, about 140 mM to about 150 mM, about 70 mM to about 140 mM, about 70 mM to about 130 mM, about 70 mM to about 120 mM, about 70 mM to about 110 mM, about 70 mM to about 100 mM, about 70 mM to about 90 mM, about 70 mM to about 80 mM, about 90 mM to about 130 mM, about 100 mM to about 120 mM, about 105 mM to about 115 mM calcium) and about 150 mM to about 230 mM glutamate (e.g., about 150 mM to about 220 mM, about 150 mM to about 210 mM, about 150 mM to about 200 mM, about 150 mM to about 190 mM, about 150 mM to about 180 mM, about 150 mM to about 170 mM, about 150 mM to about 160 mM, about 160 mM to about 230 mM, about 170 mM to about 230 mM, about 180 mM to about 230 mM, about 190 mM to about 230 mM, about 200 mM to about 230 mM, about 210 mM to about 230 mM, about 220 mM to about 230 mM, about 170 mM to about 210 mM, about 180 mM to about 200 mM, about 185 mM to about 195 mM glutamate). In exemplary aspects, the anti-TSLP antibody, e.g., tezepelumab, is formulated in about 105 mM to about 115 mM calcium and about 225 mM to about 235 mM glutamate, or about 235 to 245 mM glutamate. In exemplary embodiments, the presently disclosed composition comprises greater than 140 mg/mL tezepelumab, about 99 mM to about 121 mM calcium, 171 mM to about 209 mM glutamate, and 0.01% (w/v) polysorbate 80. In exemplary embodiments, the presently disclosed composition comprises greater than 140 mg/mL tezepelumab, about 110 mM calcium, 240 mM glutamate, and 0.01% (w/v) polysorbate 80. Optionally, the pH is about 5.0±0.2, or about 5.0±0.1.

Combined Excipients

The presently disclosed compositions in various aspects comprises more than one excipient which reduces the viscosity of the high protein concentration formulation. In various aspects, the presently disclosed composition further comprises one or more of: N-acetyl arginine (NAR), N-acetyl lysine, methionine, glycine, proline, sodium acetate, tris acetate, a histidine salt, or a calcium salt. In various aspects, the presently disclosed composition comprises (i) an arginine salt and (ii) NAR and/or methionine. In various aspects, the arginine salt is arginine glutamate. In various instances, the presently disclosed composition comprises (i) a calcium salt and (ii) NAR and/or methionine. In exemplary aspects, the calcium salt is calcium glutamate.

In various aspect, one or more of N-acetyl arginine (NAR), N-acetyl lysine, methionine, glycine, proline, sodium acetate, tris acetate, a histidine salt, and a calcium salt is present in the composition in an amount of about 15 mM to about 300 mM, or about 50 mM to about 300 mM. In exemplary aspects, the presently disclosed composition comprises one or more of N-acetyl arginine (NAR), N-acetyl lysine, methionine, glycine, proline, sodium acetate, tris acetate, a histidine salt, and a calcium salt is present in the composition in an amount of about 15 mM to about 200 mM, about 15 to about 150 mM, about 50 mM to about 250 mM, about 50 mM to about 200 mM, about 50 mM to about 150 mM, about 50 mM to about 100 mM, about 50 mM to about 90 mM, about 50 mM to about 80 mM, about 50 mM to about 70 mM, about 50 mM to about 60 mM, about 50 mM to about 55 mM, about 55 mM to about 200 mM, about 60 mM to about 200 mM, about 70 mM to about 200 mM, about 80 mM to about 200 mM, about 90 mM to about 200 mM, about 100 mM to about 200 mM, about 150 mM to about 200 mM, about 160 mM to about 200 mM, about 170 mM to about 200 mM, about 180 mM to about 200 mM, or about 190 mM to about 200 mM. In various instances, the presently disclosed composition comprises one or more of N-acetyl arginine (NAR), N-acetyl lysine, methionine, glycine, proline, sodium acetate, tris acetate, a histidine salt, and a calcium salt is present in the composition in an amount of about 50 mM to about 100 mM (e.g., about 50 mM, about 60 mM, about 70 mM, about 80 mM, about 90 mM, about 100 mM) or about 100 mM to about 200 mM (e.g., about 100 mM, about 110 mM, about 120 mM, about 130 mM, about 140 mM, about 150 mM, about 160 mM, about 170 mM, about 180 mM, about 190 mM, about 200 mM). In various instances, the presently disclosed composition comprises one or more of N-acetyl arginine (NAR), N-acetyl lysine, methionine, glycine, proline, sodium acetate, tris acetate, a histidine salt, and a calcium salt is present in the composition in an amount of about 15 mM to about 200 mM, about 50 mM to about 100 mM or about 50 mM to about 75 mM or about 75 mM to about 100 mM. In various instances, the presently disclosed composition comprises one or more of N-acetyl arginine (NAR), N-acetyl lysine, methionine, glycine, proline, sodium acetate, tris acetate, a histidine salt, and a calcium salt is present in the composition in an amount of about 100 mM to about 200 mM or about 100 mM to about 150 mM or about 150 mM to about 200 mM.

In various aspects, the amount of the basic amino acid or salt thereof, or calcium salt or magnesium salt, may be reduced when combined with each other or another viscosity-reducing excipient. In various aspects, the amount of the basic amino acid or salt thereof, or calcium salt or magnesium salt, is reduced ˜50% when combined with each other or another viscosity-reducing excipient, such as proline, compared to the formulation not comprising the other viscosity reducing excipient, e.g., proline. In exemplary instances, the viscosity-lowering excipients present in the composition are present in a total amount of about 0 mM to about 250 mM, about 0 mM to about 220 mM, about 50 mM to about 250 mM, about 50 mM to about 200 mM, about 50 mM to about 150 mM, about 50 mM to about 100 mM, about 50 mM to about 90 mM, about 50 mM to about 80 mM, about 50 mM to about 70 mM, about 50 mM to about 60 mM, about 50 mM to about 55 mM, about 55 mM to about 200 mM, about 60 mM to about 200 mM, about 70 mM to about 200 mM, about 80 mM to about 200 mM, about 90 mM to about 200 mM, about 100 mM to about 200 mM, about 150 mM to about 200 mM, about 160 mM to about 200 mM, about 170 mM to about 200 mM, about 180 mM to about 200 mM, or about 190 mM to about 200 mM.

Optionally, the compositions of the present disclosure comprise one or more of a basic amino acid, or a salt thereof, and/or a calcium salt and/or magnesium salt and/or one or more of: N-acetyl arginine (NAR), N-acetyl lysine, methionine, glycine, proline (e.g., L-proline), sodium acetate, tris acetate, a histidine salt, or a calcium salt, and are present in a total amount of about 50 mM to about 250 mM, about 50 mM to about 200 mM, about 50 mM to about 150 mM, about 50 mM to about 100 mM, about 50 mM to about 90 mM, about 50 mM to about 80 mM, about 50 mM to about 70 mM, about 50 mM to about 60 mM, about 50 mM to about 55 mM, about 55 mM to about 200 mM, about 60 mM to about 200 mM, about 70 mM to about 200 mM, about 80 mM to about 200 mM, about 90 mM to about 200 mM, about 100 mM to about 200 mM, about 150 mM to about 200 mM, about 160 mM to about 200 mM, about 170 mM to about 200 mM, about 180 mM to about 200 mM, or about 190 mM to about 200 mM. In various embodiments the compositions described herein comprise proline (e.g., L-proline), optionally in a total amount of from about 0 mM to about 250 mM, about 0 mM to about 220 mM, about 25 mM to about 200 mM, about 50 mM to about 150 mM, or about 50 mM to about 100 mM, In various embodiments, the proline is in an amount of about 40 mM, about 50 mM, about 60 mM, about 65 mM, about 70 mM, about 75 mM, about 80 mM, about 85 mM, about 90 mM, about 95 mM, about 100 mM, about 105 mM, about 110 mM, about 115 mM, about 120 mM, about 125 mM, about 130 mM, about 135 mM, about 140 mM, about 145 mM, about 150 mM, about 160 mM, about 170 mM, about 180 mM, about 190 mM, about 200 mM, about 210 mM, about 220 mM, about 230 mM, about 240 mM, or about 250 mM.

In various instances, the composition of the present disclosure comprises greater than 140 mg/mL anti-TSLP antibody, e.g., tezepelumab, a surfactant, a basic amino acid, or a salt thereof, and proline. In various instances, greater than 140 mg/mL anti-TSLP antibody, e.g., tezepelumab, is formulated with about 10 mM to 200 mM, or about 50 mM to about 150 mM basic amino acid, or a salt thereof, and about 50 mM to about 250 mM proline, or about 50 mM to about 150 mM proline. Optionally, greater than 140 mg/mL anti-TSLP antibody, e.g., tezepelumab, is formulated with about 50 mM to about 100 mM basic amino acid, or a salt thereof, and about 90 mM to about 150 mM proline. In various aspects, the salt of the basic amino acid is arginine glutamate and an amount of arginine is added and an amount of glutamate is added to arrive at a solution comprising arginine glutamate. In various aspects, greater than 140 mg/mL anti-TSLP antibody, e.g., tezepelumab, is formulated with about 40 mM to about 120 mM arginine and about 45 mM to about 125 mM glutamate and about 60 mM to about 140 mM proline. Optionally, the anti-TSLP antibody, e.g., tezepelumab is formulated with about 50 mM to about 110 mM, about 60 mM to about 100 mM, about 70 mM to about 90 mM, or about 75 mM to about 85 mM arginine, and about 55 mM to about 115 mM, about 65 mM to about 105 mM, about 75 mM to about 95 mM, about 80 mM to or about 90 mM glutamate, and about 70 mM to about 130 mM, about 80 mM to about 120 mM, about 90 mM to about 110 mM, or about 95 mM to about 105 mM proline. In various aspects, the anti-TSLP antibody is formulated in about 10 mM to about 90 mM arginine and about 55 mM to about 135 mM glutamate and about 45 mM to about 125 mM proline. Optionally, the anti-TSLP antibody, e.g., tezepelumab, is formulated in about 20 mM to about 80 mM, about 30 mM to about 70 mM, about 40 mM to about 60 mM, about 45 mM to about 55 mM arginine, and about 65 mM to about 125 mM, about 75 mM to about 115 mM, about 85 mM to about 105 mM, about 90 mM to about 100 mM glutamate, and about 55 mM to about 115 mM, about 65 mM to about 105 mM, about 75 mM to about 95 mM, about 80 mM to about 90 mM, or about 85 mM proline. In various aspects, the composition comprises greater than 140 mg/mL anti-TSLP antibody, about 10 mM to about 90 mM arginine and about 55 mM to about 135 mM glutamate and about 45 mM to about 125 mM proline. Optionally, the composition comprises the anti-TSLP antibody, e.g., tezepelumab, about 20 mM to about 80 mM, about 30 mM to about 70 mM, about 40 mM to about 60 mM, about 45 mM to about 55 mM arginine, and about 65 mM to about 125 mM, about 75 mM to about 115 mM, about 85 mM to about 105 mM, about 90 mM to about 100 mM glutamate, and about 55 mM to about 115 mM, about 65 mM to about 105 mM, about 75 mM to about 95 mM, about 80 mM to about 90 mM, about 85 mM proline. In various instances, the composition of the present disclosure comprises greater than 140 mg/mL tezepelumab and about 45 mM to about 55 mM arginine and about 85.5 mM to about 104.5 mM glutamate and about 76.5 mM to about 93.5 mM proline.

In various instances, the composition of the present disclosure comprises greater than 140 mg/mL anti-TSLP antibody, e.g., tezepelumab, a surfactant, a calcium salt or a magnesium salt, and proline, e.g., L-proline. In various instances, greater than 140 mg/mL anti-TSLP antibody, e.g., tezepelumab, is formulated with about 15 mM to about 150 mM calcium salt or magnesium salt, and about 50 mM to about 150 mM proline. Optionally, greater than 140 mg/mL anti-TSLP antibody, e.g., tezepelumab, is formulated with about 50 mM to about 100 mM basic amino acid, or a salt thereof, and about 90 mM to about 150 mM proline. In various aspects, the calcium is calcium glutamate and an amount of calcium is added and an amount of glutamate is added to arrive at a solution comprising calcium glutamate. In various instances, the anti-TSLP antibody is formulated with about 20 mM to about 100 mM calcium and about 100 mM to about 180 mM glutamate and about 30 mM to about 110 mM proline. Optionally, the anti-TSLP antibody, e.g., tezepelumab, is formulated with about 30 mM to about 90 mM, about 40 mM to about 80 mM, about 50 mM to about 70 mM, about 55 mM to about 65 mM, or about 60 mM calcium, and about 110 mM to about 170 mM, about 120 mM to about 160 mM, about 130 mM to about 150 mM, about 135 mM to about 145 mM, or about 140 mM glutamate, and about 40 mM to about 100 mM, about 50 mM to about 90 mM, about 60 mM to about 80 mM, about 65 mM to about 75 mM, or about 70 mM proline. In various instances, the anti-TSLP antibody is formulated in about 30 mM to about 110 mM calcium, about 105 mM to about 185 mM glutamate, and about 20 mM to about 100 mM proline. Optionally, the anti-TSLP antibody, e.g., tezepelumab is formulated in about 40 mM to about 100 mM, about 50 mM to about 90 mM, about 60 mM to about 80 mM, about 65 mM to about 75 mM, or about 75 mM calcium, and about 115 mM to about 175 mM, about 125 mM to about 165 mM, about 135 mM to about 155 mM, about 140 mM to about 150 mM, or about 145 mM glutamate, and about 30 mM to about 90 mM, about 40 mM to about 80 mM, about 50 mM to about 70 mM, about 55 mM to about 65 mM, or about 60 mM proline. In various instances, the composition comprises greater than 140 mg/mL anti-TSLP antibody, about 30 mM to about 110 mM calcium, about 105 mM to about 185 mM glutamate, and about 20 mM to about 100 mM proline. Optionally, the composition comprises the anti-TSLP antibody, e.g., tezepelumab, about 40 mM to about 100 mM, about 50 mM to about 90 mM, about 60 mM to about 80 mM, about 65 mM to about 75 mM, or about 75 mM calcium, and about 115 mM to about 175 mM, about 125 mM to about 165 mM, about 135 mM to about 155 mM, about 140 mM to about 150 mM, or about 145 mM glutamate, and about 30 mM to about 90 mM, about 40 mM to about 80 mM, about 50 mM to about 70 mM, about 55 mM to about 65 mM, or about 60 mM proline. In various instances, the composition of the present disclosure comprises greater than 140 mg/mL tezepelumab and about 63 mM to about 77 mM calcium and about 130.5 mM to about 159.5 mM glutamate and about 54 mM to about 66 mM proline.

Surfactant

The compositions of the present disclosure in various aspects comprise a surfactant. Surfactants are surface active agents that are amphipathic (having a polar head and hydrophobic tail). Surfactants preferentially accumulate at interfaces, resulting in reduced interfacial tension. Use of a surfactant can also help to mitigate formation of large proteinaceous particles. In some aspects, the surfactant present in the compositions of the present disclosure is an amphipathic and/or nonionic surfactant. Exemplary surfactants include polyoxyethylene sorbitan fatty acid esters (e.g. polysorbate 20, polysorbate 80), alkylaryl polyethers, e.g. oxyethylated alkyl phenol (e.g. Triton™ X-100), and poloxamers (e.g. Pluronics®, e.g. Pluronic® F68), and combinations of any of the foregoing, either within a class of surfactants or among classes of surfactants. Polysorbate 20 and polysorbate 80 (and optionally mixtures thereof) are particularly contemplated. The surfactant in exemplary instances is present in the composition at a concentration of less than or about 0.015% (w/v)±0.005% (w/v). For instance, the formulation may comprise about 0.005% (w/v) to about 0.015% (w/v) surfactant, e.g., about 0.005% (w/v), about 0.006% (w/v), about 0.007% (w/v), about 0.008% (w/v), about 0.009% (w/v), about 0.010% (w/v), about 0.011% (w/v), about 0.012% (w/v), about 0.013% (w/v), about 0.014% (w/v), about 0.015% (w/v). In exemplary aspects, the formulation comprises about 0.005% (w/v), 0.010% (w/v), or 0.015% (w/v) surfactant. In various aspects, the surfactant is a polysorbate, e.g., polysorbate 20 or polysorbate 80 or a mixture thereof. Optionally, the surfactant is present at a concentration less than or about 0.005% (w/v) to about 0.015% (w/v), optionally, about 0.010% (w/v)±0.0025% (w/v) surfactant, e.g., about 0.005% (w/v), 0.010% (w/v), or 0.015% (w/v) surfactant.

Antibody Concentration

In exemplary aspects, the presently disclosed composition comprises the anti-TSLP antibody at a concentration greater than about 100 mg/mL and less than about 300 mg/mL or less than about 250 mg/mL, optionally, at about 160 mg/mL to about 250 mg/mL, e.g., about 180 mg/mL to about 225 mg/mL, or about 180 mg/mL to about 200 mg/mL. In some aspects, the anti-TSLP antibody is present in the composition at a concentration of about 160 mg/mL to about 250 mg/mL, about 160 mg/mL to about 240 mg/mL, about 160 mg/mL to about 230 mg/mL, about 160 mg/mL to about 220 mg/mL, about 160 mg/mL to about 210 mg/mL, about 160 mg/mL to about 200 mg/mL, about 160 mg/mL to about 190 mg/mL, about 160 mg/mL to about 180 mg/mL, about 160 mg/mL to about 170 mg/mL, about 160 mg/mL to about 165 mg/mL, about 165 mg/mL to about 250 mg/mL, about 170 mg/mL to about 250 mg/mL, about 180 mg/mL to about 250 mg/mL, about 190 mg/mL to about 200 mg/mL, about 210 mg/mL to about 250 mg/mL, about 220 mg/mL to about 250 mg/mL, about 230 mg/mL to about 250 mg/mL, about 240 mg/mL to about 250 mg/mL. In some aspects, the anti-TSLP antibody is present in the composition at a concentration of about 180 mg/mL or about 190 mg/mL, about 200 mg/mL or about 210 mg/mL.

In exemplary aspects, the composition comprises about 160 mg/mL to about 250 mg/mL of an anti-TSLP antibody, optionally, about 160 mg/mL to about 225 mg/mL or about 160 mg/mL to about 200 mg/mL. In various aspects, the composition comprises about 175 mg/mL to about 185 mg/mL of an anti-TSLP antibody, optionally, about 175 mg/mL, about 176 mg/mL, about 177 mg/mL, about 178 mg/mL, about 179 mg/mL, about 180 mg/mL, about 181 mg/mL, about 182 mg/mL, about 183 mg/mL, about 184 mg/mL, about 185 mg/mL). In various aspects, the composition comprises about 180 mg/mL anti-TSLP antibody. In various instances, the concentration of the anti-TSLP antibody is about 189 mg/mL or about 190 mg/mL to about 230 mg/mL or about 231 mg/mL. Optionally, the concentration of the anti-TSLP antibody is about 205 mg/mL to about 215 mg/mL, optionally, about 210 mg/mL or about 205 mg/mL, about 206 mg/mL, about 207 mg/mL, about 208 mg/mL, about 209 mg/mL, about 210 mg/mL, about 211 mg/mL, about 212 mg/mL, about 213 mg/mL, about 214 mg/mL, about 215 mg/mL.

In exemplary aspects, the anti-TSLP antibody is present in the composition at a concentration of about 140 mg/mL to about 210 mg/mL, e.g., about 180 mg/mL±10%, about 200 mg/mL±10%, about 210 mg/mL±10%.

Additional Excipients

In exemplary aspects, the composition of the present disclosure may comprise additional components. The composition, in various aspects, comprises any pharmaceutically acceptable ingredient, including, for example, acidifying agents, additives, adsorbents, aerosol propellants, air displacement agents, alkalizing agents, anticaking agents, anticoagulants, antimicrobial preservatives, antioxidants, antiseptics, bases, binders, buffering agents, chelating agents, coating agents, coloring agents, desiccants, detergents, diluents, disinfectants, disintegrants, dispersing agents, dissolution enhancing agents, dyes, emollients, emulsifying agents, emulsion stabilizers, fillers, film forming agents, flavor enhancers, flavoring agents, flow enhancers, gelling agents, granulating agents, humectants, lubricants, mucoadhesives, ointment bases, ointments, oleaginous vehicles, organic bases, pastille bases, pigments, plasticizers, polishing agents, preservatives, sequestering agents, skin penetrants, solubilizing agents, solvents, stabilizing agents, suppository bases, surface active agents, surfactants, suspending agents, sweetening agents, therapeutic agents, thickening agents, tonicity agents, toxicity agents, viscosity-increasing agents, water-absorbing agents, water-miscible cosolvents, water softeners, or wetting agents. See, e.g., the Handbook of Pharmaceutical Excipients, Third Edition, A. H. Kibbe (Pharmaceutical Press, London, U K, 2000), which is incorporated by reference in its entirety. Remington's Pharmaceutical Sciences, Sixteenth Edition, E. W. Martin (Mack Publishing Co., Easton, Pa., 1980), which is incorporated by reference in its entirety.

pH, Viscosity, and Osmolality

In alternative aspects, the composition of the present disclosure is a liquid. In certain aspects, the liquid has a pH which is less than about 6.0, optionally, less than about 5.7, or less than about 5.5. In some aspects, the pH is about 4.5 to about 5.7, about 4.5 to about 5.5, about 4.7 to about 5.3, about 4.8 to about 5.4, or about 5.0 to about 5.7 e.g., about 4.7, about 4.8, about 4.9, about 5.0, about 5.1, about 5.2, about 5.3, about 5.4, about 5.5, about 5.6, about 5.7. In some aspects, the pH is about 4.9, 5.0, 5.1, 5.2, 5.3, 5.4, 5.5, 5.6 or 5.7. In exemplary instances, the presently disclosed composition has a pH of about 4.5 to about 6.75, optionally, about 4.8 to about 6.0.

In some aspects, the composition is characterized by a reduced viscosity, relative to a liquid composition not comprising proline. In exemplary instances, the composition is characterized by a viscosity of less than about 24 centiPoise (cP) at 23° C. when the concentration of the anti-TSLP antibody is less than 155 mg/mL, optionally, ˜6 cP when the concentration of the anti-TSLP antibody is about 110 mg/mL or about 15 cP when the concentration of the anti-TSLP antibody is about 140 mg/mL. In certain aspects, the composition is characterized by a viscosity of about 5 cP to about 20 cP, e.g., about 5 cP to about 15 cP, about 5 cP to about 10 cP, about 10 cP to about 20 cP, about 15 cP to about 20 cP, or about 5 cP, about 6 cP, about 7 cP, about 8 cP, about 9 cP, about 10 cP, about 11 cP, about 12 cP, about 13 cP, about 14 cP, about 15 cP, about 16 cP, about 17 cP, about 18 cP, about 19 cP, about 20 cP, about 21 cP, about 22 cP, when the concentration of the anti-TSLP antibody is less than 155 mg/mL (e.g., about 110 mg/mL, about 140 mg/mL).

In certain aspects, the composition is characterized by a viscosity of about 5 cP to about 25 cP, e.g., about 5 cP to about 20 cP, about 5 cP to about 15 cP, about 5 cP to about 10 cP, about 10 cP to about 25 cP, about 15 cP to about 20 cP, or about 5 cP, about 6 cP, about 7 cP, about 8 cP, about 9 cP, about 10 cP, about 11 cP, about 12 cP, about 13 cP, about 14 cP, about 15 cP, about 16 cP, about 17 cP, about 18 cP, about 19 cP, about 20 cP, about 21 cP, about 22 cP, about 23 cP, about 24 cP, about 25 cP, when the concentration of the anti-TSLP antibody is 180 mg/ml or greater (e.g., about 180 mg/ml, about 210 mg/mL, about 240 mg/mL). In exemplary aspects, the composition has a viscosity that is about 15 cP±5 cP or about 20 cP±5 cP when the concentration of the antibody is about 100 mg/mL to about 180 mg/mL. Unless noted otherwise, all viscosities disclosed herein refers to a viscosity measured using a rotational viscometer at 23° C. and at a shear rate of about 1000 1/s.

In exemplary aspects, the viscosity of the presently disclosed composition is less than 100 cP at 23° C., 1000 s⁻¹, optionally, less than 75 cP at 23° C., 1000 s⁻¹, e.g., less than 60 cP or less than 50 cP.

In exemplary aspects, the composition is intended for subcutaneous administration to a subject, and thus the composition is isotonic with the intended site of administration. For example, the osmolality of the composition is in some aspects, in a range of about 270 to about 350 mOsm/kG, or about 285 to about 345 mOsm/kG, or about 300 to about 315 mOsm/kG. For example, if the solution is in a form intended for administration parenterally, it can be isotonic with blood (about 300 mOsm/kG osmolality). In exemplary aspects, the aqueous pharmaceutical formulation has an osmolality in a range of about 200 mOsm/kg to about 500 mOsm/kg, or about 225 mOsm/kg to about 400 mOsm/kg, or about 250 mOsm/kg to about 350 mOsm/kg. Optionally, the composition is isotonic or has an osmolality greater than about 350 mOsm/kg.

Stability

In various instances, less than about 5% of the antibody is degraded after storage at about 2° C. to about 8° C. for at least or about 12 months, as determined by Size Exclusion Chromatography (SEC). In various aspects, less than about 5% of the antibody is degraded after storage at about 2° C. to about 8° C. for about 20 months to about 26 months, as determined by Size Exclusion Chromatography (SEC). In exemplary instances, less than about 5% of the antibody is degraded after storage at about 2° C. to about 8° C. for about 30 to about 40 months, as determined by Size Exclusion Chromatography (SEC). In exemplary instances, less than about 5% of the antibody is degraded after storage at about 2° C. to about 8° C. for about 2 years to about 3 years, as determined by Size Exclusion Chromatography (SEC). Also, for example, less than 5% of the antibody is degraded after about 24 months to about 36 months of storage at 2° C. to 8° C. as determined by Size Exclusion Chromatography (SEC), optionally, wherein less than 2% of the antibody is degraded after 24 months or 36 months of storage at 2° C. to 8° C. In various aspects, less than 5% of the antibody is degraded after at least 2 weeks (optionally, after at least 1 month, after at least 2 months, after at least 3 months, after at least 4 months, after at least 5 months or after at least 6 months) of storage at about room temperature (e.g., 25° C.), as determined by SEC. In various instances, less than 5% of the antibody is degraded after about 24 months to about 36 months of storage at 2° C. to 8° C. followed by at least 2 weeks or at least about 1 month or at least about 2 months of storage at about room temperature (e.g., 25° C.), as determined by SEC. Optionally, less than about 5% of the antibody is degraded after storage at a temperature greater than about 20° C. for at least or about 2 weeks, as determined by Size Exclusion Chromatography (SEC), optionally, for at least or about 4 weeks or about 8 weeks. In various aspects, the temperature is greater than or about 25° C. or greater than or about 30° C. or greater than or about 40° C.

Articles of Manufacture, Syringes, and Vials

An article of manufacture is further provided herein. In exemplary embodiments, the article comprises the composition of the present disclosure, optionally, comprising about 1 mL to about 5 mL, e.g., about 1 mL to about 3 mL of the aqueous composition. In exemplary aspects of the disclosure, the composition is provided for storage or use, e.g. in a single-use vial, single-use syringe, or glass, glass-lined, glass-coated primary container or auto-injector. In exemplary aspects, the composition is provided in a single use system bag or a polycarbonate carboy for frozen storage. In alternative aspects, the composition is contained in glass vials or syringes for storage at 2° C. to 8° C. A pre-filled syringe comprising the presently disclosed composition, optionally, comprising about 1 mL to about 5 mL, e.g., about 1 mL to about 3 mL of the composition, is additionally provided herein. Further provided is a vial comprising the presently disclosed composition, optionally, comprising about 1 mL to about 5 mL, e.g., about 1 mL to about 3 mL of the aqueous composition. In various aspects, the article, prefilled syringe, or vial comprises about 2 mL to about 3 mL, e.g., about 2.1 mL, about 2.2 mL, about 2.3 mL, about 2.4 mL, about 2.5 mL, about 2.6 mL, about 2.7 mL, about 2.8 mL, about 2.9 mL, of the composition of the present disclosure and in various aspects, the composition comprises tezepelumab at a concentration of about 180 mg/mL to provide about 420 mg tezepelumab.

In exemplary instances, the composition is provided for use in a delivery system which is off-the-shelf and/or designed for self-administration. In exemplary aspects, the composition is provided in a pre-filled syringe or an autoinjector, a pen injector, a dual-chamber pen, and the like. Such products are known in the art and are commercially available. See, e.g., Shire, Steven, Monoclonal Antibodies: Meeting the Challenges in Manufacturing, Formulation, Delivery and Stability of Final Drug Product, Chapter 8: Development of delivery device technology to deal with the challenges of highly viscous mAb formulations at high concentration, Woodhead Publishing, Cambridge, UK, pages 153-162 (2015). In exemplary aspects, the composition is provided for use in an YpsoMate™ autoinjection, an YpsoMate™ 2.25 autoinjector, or a VarioJect™ (YpsoMed, Burgdorf, Switzerland). Other autoinjectors include, e.g., Self Dose™ Patient-Controlled Injector, BD Physioject™ disposable autoinjector, Autoject® II Syringe Injector (Owen Mumford, Oxfordshire, UK). In various embodiments, the auto-injector is an Ypsomed YpsoMate® auto-injector. Additional auto-injectors contemplated for in the methods are disclosed in International Patent Publications WO 2018/226565, WO 2019/094138, WO 2019/178151, WO 20120/072577, WO2020/081479, WO 2020/081480, and International Patent Application Nos. PCT/US20/70590, PCT/US20/70591, PCT/US20/53180, PCT/US20/53179, PCT/US20/53178, and PCT/US20/53176, incorporated by reference herein.

The composition of the present disclosure can be suitable for administration by any acceptable route, including parenteral, and specifically subcutaneous. For example, the subcutaneous administration can be to the upper arm, upper thigh, or abdomen. Other routes include intravenous, intradermal, intramuscular, intraperitoneal, intranodal and intrasplenic, for example. The subcutaneous route is preferred.

If the composition is in a form intended for administration to a subject, it can be made to be isotonic with the intended site of administration. For example, if the solution is in a form intended for administration parenterally, it can be isotonic with blood. The composition typically is sterile. In certain embodiments, this may be accomplished by filtration through sterile filtration membranes. In certain embodiments, parenteral compositions generally are placed into a container having a sterile access port, for example, an intravenous solution bag, or vial having a stopper pierceable by a hypodermic injection needle, or a prefilled syringe. In certain embodiments, the composition may be stored in a ready-to-use form.

Anti-TSLP Antibodies

The composition of the present disclosure comprises an anti-TSLP antibody. In exemplary embodiments, the anti-TSLP antibody specifically binds to a TSLP polypeptide as set forth in amino acids 29-159 of SEQ ID NO: 2. Thymic stromal lymphopoietin (TSLP) is an epithelial cell-derived cytokine that is produced in response to pro-inflammatory stimuli and drives allergic inflammatory responses primarily through its activity on dendritic cells (Gilliet, J Exp Med. 197:1059-1067, 2003; Soumelis, Nat Immunol. 3:673-680, 2002; Reche, J Immunol. 167:336-343, 2001), mast cells (Allakhverdi, J Exp Med. 204:253-258, 2007) and CD34+ progenitor cells. Swedin et al., Pharmacol Ther 169: 13-34 (2017). TSLP signals through a heterodimeric receptor consisting of the interleukin (IL)-7 receptor alpha (IL-7Rα) chain and a common γ chain-like receptor (TSLPR) (Pandey, Nat Immunol. 1:59-64, 2000; Park, J Exp Med. 192:659-669, 2000).

Human TSLP mRNA (Brightling et al., J Allergy Clin Immunol 121:5-10 quiz 1-2 (2008); Ortega et al., NEJM 371:1198-1207 (2014)) and protein levels (Ortega et al., (2014), supra) are increased in the airways of asthmatic individuals compared to controls, and the magnitude of this expression correlates with disease severity. Brightling et al, (2008), supra. Recent studies have demonstrated association of a single nucleotide polymorphism in the human TSLP locus with protection from asthma, atopic asthma and airway hyperresponsiveness, suggesting that differential regulation of TSLP gene expression might influence disease susceptibility. (To et al., BMC Public Health 12: 204 (2012); XOLAIR® (omalizumab): Highlights of Prescribing Information 2016. (at https://www.gene.com/download/pdf/xolair_prescribing.pdf); Bleecker et al., The Lancet 388: 2115-2127 (2016). These data suggest that targeting TSLP may inhibit multiple biological pathways involved in asthma.

Earlier non-clinical studies of TSLP suggested that after TSLP is released from airway epithelial cells or stromal cells, it activates mast cells, dendritic cells, and T cells to release Th2 cytokines (e.g., IL-4/13/5). Recently published human data demonstrated a good correlation between tissue TSLP gene and protein expression, a Th2 gene signature score, and tissue eosinophils in severe asthma. Therefore, an anti-TSLP target therapy may be effective in asthmatic patients with Th2-type inflammation (Shikotra et al, J Allergy Clin Immunol. 129(1):104-11, 2012).

Data from other studies suggest that TSLP may promote airway inflammation through Th2 independent pathways such as the crosstalk between airway smooth muscle and mast cells (Allakhverdi et al, J Allergy Clin Immunol. 123(4):958-60, 2009; Shikotra et al, supra). TSLP can also promote induction of T cells to differentiate into Th-17-cytokine producing cells with a resultant increase in neutrophilic inflammation commonly seen in more severe asthma (Tanaka et al, Clin Exp Allergy. 39(1):89-100, 2009). These data and other emerging evidence suggest that blocking TSLP may serve to suppress multiple biologic pathways including but not limited to those involving Th2 cytokines (IL-4/13/5).

It is contemplated that antibodies specific for TSLP are useful in the treatment of asthma, including severe asthma, eosinophlic asthma, no-eosinophilic/low-eosinophilic and other forms of asthma described herein.

Specific binding agents such as antibodies and antibody variants or fragments that bind to their target antigen, e.g., TSLP, are useful in the methods of the disclosure. In one embodiment, the specific binding agent is an antibody. The antibodies may be monoclonal (MAbs); recombinant; chimeric; humanized, such as complementarity-determining region (CDR)-grafted; human; antibody variants, including single chain; and/or bispecific; as well as fragments; variants; or derivatives thereof. Antibody fragments include those portions of the antibody that bind to an epitope on the polypeptide of interest. Examples of such fragments include Fab and F(ab′) fragments generated by enzymatic cleavage of full-length antibodies. Other binding fragments include those generated by recombinant DNA techniques, such as the expression of recombinant plasmids containing nucleic acid sequences encoding antibody variable regions.

Monoclonal antibodies may be modified for use as therapeutics or diagnostics. One embodiment is a “chimeric” antibody in which a portion of the heavy (H) and/or light (L) chain is identical with or homologous to a corresponding sequence in antibodies derived from a particular species or belonging to a particular antibody class or subclass, while the remainder of the chain(s) is/are identical with or homologous to a corresponding sequence in antibodies derived from another species or belonging to another antibody class or subclass. Also included are fragments of such antibodies, so long as they exhibit the desired biological activity. See U.S. Pat. No. 4,816,567; Morrison et al., 1985, Proc. Natl. Acad. Sci. 81:6851-55.

In another embodiment, a monoclonal antibody is a “humanized” antibody. Methods for humanizing non-human antibodies are well known in the art. See U.S. Pat. Nos. 5,585,089 and 5,693,762. Generally, a humanized antibody has one or more amino acid residues introduced into it from a source that is non-human. Humanization can be performed, for example, using methods described in the art (Jones et al., 1986, Nature 321:522-25; Riechmann et al., 1998, Nature 332:323-27; Verhoeyen et al., 1988, Science 239:1534-36), by substituting at least a portion of a rodent complementarity-determining region for the corresponding regions of a human antibody.

Also encompassed by the disclosure are human antibodies and antibody variants (including antibody fragments) that bind TSLP. Using transgenic animals (e.g., mice) that are capable of producing a repertoire of human antibodies in the absence of endogenous immunoglobulin production such antibodies are produced by immunization with a polypeptide antigen (i.e., having at least 6 contiguous amino acids), optionally conjugated to a carrier. See, e.g., Jakobovits et al., 1993, Proc. Natl. Acad. Sci. 90:2551-55; Jakobovits et al., 1993, Nature 362:255-58; Bruggermann et al., 1993, Year in Immuno. 7:33. See also PCT App. Nos. PCT/US96/05928 and PCT/US93/06926. Additional methods are described in U.S. Pat. No. 5,545,807, PCT App. Nos. PCT/US91/245 and PCT/GB89/01207, and in European Patent Nos. 546073B1 and 546073A1. Human antibodies can also be produced by the expression of recombinant DNA in host cells or by expression in hybridoma cells as described herein.

Chimeric, CDR grafted, and humanized antibodies and/or antibody variants are typically produced by recombinant methods. Nucleic acids encoding the antibodies are introduced into host cells and expressed using materials and procedures described herein. In a preferred embodiment, the antibodies are produced in mammalian host cells, such as CHO cells. Monoclonal (e.g., human) antibodies may be produced by the expression of recombinant DNA in host cells or by expression in hybridoma cells as described herein.

Antibodies and antibody variants (including antibody fragments) useful in the present methods comprise an anti-TSLP antibody comprising (A) a light chain variable domain comprising: (i) a light chain CDR1 sequence comprising the amino acid sequence set forth in SEQ ID NO:3; (ii) a light chain CDR2 sequence comprising the amino acid sequence set forth in SEQ ID NO:4; and (iii) a light chain CDR3 sequence comprising the amino acid sequence set forth in SEQ ID NO:5; and (B) a heavy chain variable domain comprising: (i) a heavy chain CDR1 sequence comprising the amino acid sequence set forth in SEQ ID NO:6; (ii) a heavy chain CDR2 sequence comprising the amino acid sequence set forth in SEQ ID NO:7, and (iii) a heavy chain CDR3 sequence comprising the amino acid sequence set forth in SEQ ID NO:8.

Also contemplated is an antibody or antibody variant comprising (A) a light chain variable domain selected from the group consisting of: (i) a sequence of amino acids at least 80% (e.g., about 85%, about 90%, about 95%, greater than 95%) identical to SEQ ID NO:12; (ii) a sequence of amino acids encoded by a polynucleotide sequence that is at least 80% (e.g., about 85%, about 90%, about 95%, greater than 95%) identical to SEQ ID NO:11; (iii) a sequence of amino acids encoded by a polynucleotide that hybridizes under moderately stringent conditions to the complement of a polynucleotide consisting of SEQ ID NO:11; and (B) a heavy chain variable domain selected from the group consisting of: (i) a sequence of amino acids that is at least 80% (e.g., about 85%, about 90%, about 95%, greater than 95%) identical to SEQ ID NO:10; (ii) a sequence of amino acids encoded by a polynucleotide sequence that is at least 80% (e.g., about 85%, about 90%, about 95%, greater than 95%) identical to SEQ ID NO:9; (iii) a sequence of amino acids encoded by a polynucleotide that hybridizes under moderately stringent conditions to the complement of a polynucleotide consisting of SEQ ID NO:9; or (C) a light chain variable domain of (A) and a heavy chain variable domain of (A), wherein the antibody or antibody variant specifically binds to a TSLP polypeptide as set forth in amino acids 29-159 of SEQ ID NO:2.

In exemplary instances, the anti-TSLP antibody comprises a heavy chain comprising the amino acid sequence of SEQ ID NO: 13, a light chain comprising the amino acid sequence of SEQ ID NO: 14, or a heavy chain comprising the amino acid sequence of SEQ ID NO: 13 and a light chain comprising the amino acid sequence of SEQ ID NO: 14.

Tezepelumab is an exemplary anti-TSLP antibody having (A) a light chain variable domain comprising: (i) a light chain CDR1 sequence comprising the amino acid sequence set forth in SEQ ID NO:3; (ii) a light chain CDR2 sequence comprising the amino acid sequence set forth in SEQ ID NO:4; and (iii) a light chain CDR3 sequence comprising the amino acid sequence set forth in SEQ ID NO:5; and (B) a heavy chain variable domain comprising: (i) a heavy chain CDR1 sequence comprising the amino acid sequence set forth in SEQ ID NO:6; (ii) a heavy chain CDR2 sequence comprising the amino acid sequence set forth in SEQ ID NO:7, and (iii) a heavy chain CDR3 sequence comprising the amino acid sequence set forth in SEQ ID NO:8.

Tezepelumab also comprises:

(A) a light chain variable domain selected from the group consisting of:
(i) a sequence of amino acids at least 80% identical to SEQ ID NO:12;
(ii) a sequence of amino acids encoded by a polynucleotide sequence that is at least 80% identical to SEQ ID NO:11;
(iii) a sequence of amino acids encoded by a polynucleotide that hybridizes under moderately stringent conditions to the complement of a polynucleotide consisting of SEQ ID NO:11; and

(B) a heavy chain variable domain selected from the group consisting of:
(i) a sequence of amino acids that is at least 80% identical to SEQ ID NO:10;
(ii) a sequence of amino acids encoded by a polynucleotide sequence that is at least 80% identical to SEQ ID NO:9;
(iii) a sequence of amino acids encoded by a polynucleotide that hybridizes under moderately stringent conditions to the complement of a polynucleotide consisting of SEQ ID NO:9; or

(C) a light chain variable domain of (A) and a heavy chain variable domain of (B).

Other exemplary anti-TSLP antibodies are known in the art. See, e.g., International Patent Application Publication Nos. WO2017/042701, WO2016/142426, WO2010/017468, U.S. Patent Application Publication No. US2012/0020988, and U.S. Pat. No. 8,637,019. In exemplary aspects, the anti-TSLP antibody is an antibody disclosed in one of these publications.

In various embodiments, the anti-TSLP antibody or antibody variant thereof is bivalent and selected from the group consisting of a human antibody, a humanized antibody, a chimeric antibody, a monoclonal antibody, a recombinant antibody, an antigen-binding antibody fragment, a single chain antibody, a monomeric antibody, a diabody, a triabody, a tetrabody, a Fab fragment, an IgG1 antibody, an IgG2 antibody, an IgG3 antibody, and an IgG4 antibody. In exemplary aspects, the anti-TSLP antibody is an IgG2 antibody.

In various embodiments, the anti-TSLP antibody variant is selected from the group consisting of a diabody, a triabody, a tetrabody, a Fab fragment, single domain antibody, scFv, wherein the dose is adjusted such that the binding sites to be equimolar to the those dosed by bivalent antibodies. In exemplary aspects, both binding sites of the antibody have identical binding to TSLP.

It is contemplated that the antibody or antibody variant is an IgG2 antibody. Exemplary sequences for a human IgG2 constant region are available from the Uniprot database as Uniprot number P01859, incorporated herein by reference. Information, including sequence information for other antibody heavy and light chain constant regions is also publicly available through the Uniprot database as well as other databases well-known to those in the field of antibody engineering and production.

In certain embodiments, derivatives of antibodies include tetrameric glycosylated antibodies wherein the number and/or type of glycosylation site has been altered compared to the amino acid sequences of a parent polypeptide. In certain embodiments, variants comprise a greater or a lesser number of N-linked glycosylation sites than the native protein. Alternatively, substitutions which eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created. Additional preferred antibody variants include cysteine variants wherein one or more cysteine residues are deleted from or substituted for another amino acid (e.g., serine) as compared to the parent amino acid sequence. Cysteine variants may be useful when antibodies must be refolded into a biologically active conformation such as after the isolation of insoluble inclusion bodies. Cysteine variants generally have fewer cysteine residues than the native protein, and typically have an even number to minimize interactions resulting from unpaired cysteines.

Desired amino acid substitutions (whether conservative or non-conservative) can be determined by those skilled in the art at the time such substitutions are desired. In certain embodiments, amino acid substitutions can be used to identify important residues of antibodies to human TSLP, or to increase or decrease the affinity of the antibodies to human TSLP described herein.

According to certain embodiments, preferred amino acid substitutions are those which: (1) reduce susceptibility to proteolysis, (2) reduce susceptibility to oxidation, (3) alter binding affinity for forming protein complexes, (4) alter binding affinities, and/or (4) confer or modify other physiochemical or functional properties on such polypeptides. According to certain embodiments, single or multiple amino acid substitutions (in certain embodiments, conservative amino acid substitutions) may be made in the naturally-occurring sequence (in certain embodiments, in the portion of the polypeptide outside the domain(s) forming intermolecular contacts). In certain embodiments, a conservative amino acid substitution typically may not substantially change the structural characteristics of the parent sequence (e.g., a replacement amino acid should not tend to break a helix that occurs in the parent sequence, or disrupt other types of secondary structure that characterizes the parent sequence). Examples of art-recognized polypeptide secondary and tertiary structures are described in Proteins, Structures and Molecular Principles (Creighton, Ed., W. H. Freeman and Company, New York (1984)); Introduction to Protein Structure (C. Branden and J. Tooze, eds., Garland Publishing, New York, N.Y. (1991)); and Thornton et al. Nature 354:105 (1991), which are each incorporated herein by reference.

Consistent with the foregoing, in some aspects, the composition of the present disclosure comprises about 110 mg/mL to about 140 mg/mL anti-TSLP antibody, about 0.01% (w/v)±0.005% (w/v) polysorbate 80, greater than about 2.5% (w/v) and less than about 3.0% (w/v) L-proline, and about 20 mM to about 30 mM acetate, wherein the viscosity of the composition is less than about 20 cP (e.g., 15 cP) at 23° C. and the pH is less than about 5.5, optionally, about 5.2. Optionally, the anti-TSLP antibody comprises (A) a light chain variable domain comprising: (i) a light chain CDR1 sequence comprising the amino acid sequence set forth in SEQ ID NO:3; (ii) a light chain CDR2 sequence comprising the amino acid sequence set forth in SEQ ID NO:4; and (iii) a light chain CDR3 sequence comprising the amino acid sequence set forth in SEQ ID NO:5; and (B) a heavy chain variable domain comprising: (i) a heavy chain CDR1 sequence comprising the amino acid sequence set forth in SEQ ID NO:6; (ii) a heavy chain CDR2 sequence comprising the amino acid sequence set forth in SEQ ID NO:7, and (iii) a heavy chain CDR3 sequence comprising the amino acid sequence set forth in SEQ ID NO:8. In exemplary instances, the composition comprises about 110 mg/mL of an anti-TSLP antibody, e.g., tezepelumab, 0.01% (w/v) polysorbate 80, about 2.5% (w/v) to about 3.0% (w/v) L-proline, and about 20 mM to about 22 mM acetate, wherein the composition has a pH of about 5.2, wherein the anti-TSLP antibody optionally comprises (A) a light chain variable domain comprising: (i) a light chain CDR1 sequence comprising the amino acid sequence set forth in SEQ ID NO:3; (ii) a light chain CDR2 sequence comprising the amino acid sequence set forth in SEQ ID NO:4; and (iii) a light chain CDR3 sequence comprising the amino acid sequence set forth in SEQ ID NO:5; and (B) a heavy chain variable domain comprising: (i) a heavy chain CDR1 sequence comprising the amino acid sequence set forth in SEQ ID NO:6; (ii) a heavy chain CDR2 sequence comprising the amino acid sequence set forth in SEQ ID NO:7, and (iii) a heavy chain CDR3 sequence comprising the amino acid sequence set forth in SEQ ID NO:8. In alternative instances, the composition comprises about 140 mg/mL of an anti-TSLP antibody, e.g., tezepelumab, 0.01% (w/v) polysorbate 80, about 2.6% (w/v) to about 2.7% (w/v) L-proline, and about 23 mM to about 25 mM acetate, wherein the composition has a pH of about 5.2, wherein the anti-TSLP antibody optionally comprises (A) a light chain variable domain comprising: (i) a light chain CDR1 sequence comprising the amino acid sequence set forth in SEQ ID NO:3; (ii) a light chain CDR2 sequence comprising the amino acid sequence set forth in SEQ ID NO:4; and (iii) a light chain CDR3 sequence comprising the amino acid sequence set forth in SEQ ID NO:5; and (B) a heavy chain variable domain comprising: (i) a heavy chain CDR1 sequence comprising the amino acid sequence set forth in SEQ ID NO:6; (ii) a heavy chain CDR2 sequence comprising the amino acid sequence set forth in SEQ ID NO:7, and (iii) a heavy chain CDR3 sequence comprising the amino acid sequence set forth in SEQ ID NO:8.

Consistent with the foregoing, in some aspects, the composition of the present disclosure comprises about 180 mg/mL to about 210 mg/mL anti-TSLP antibody, about 0.01% (w/v)±0.005% (w/v) polysorbate 80, 15-190 mM arginine base, 25-200 mM glutamic acid and 0-250 mM proline, wherein the viscosity of the composition is less than about 22 cP (e.g., 15, 17 or 20 cP) at 23° C. and the pH is less than about 5.7, optionally, about 5.4. Optionally, the anti-TSLP antibody comprises (A) a light chain variable domain comprising: (i) a light chain CDR1 sequence comprising the amino acid sequence set forth in SEQ ID NO:3; (ii) a light chain CDR2 sequence comprising the amino acid sequence set forth in SEQ ID NO:4; and (iii) a light chain CDR3 sequence comprising the amino acid sequence set forth in SEQ ID NO:5; and (B) a heavy chain variable domain comprising: (i) a heavy chain CDR1 sequence comprising the amino acid sequence set forth in SEQ ID NO:6; (ii) a heavy chain CDR2 sequence comprising the amino acid sequence set forth in SEQ ID NO:7, and (iii) a heavy chain CDR3 sequence comprising the amino acid sequence set forth in SEQ ID NO:8. In various embodiments, the aqueous composition comprises 140 mM arginine base and 150 mM glutamic acid. In various embodiments, the aqueous composition comprises 140 mM Arginine base, 150 mM glutamic acid, 0.01% (w/v) Polysorbate 80, pH 5.4. In various embodiments, the aqueous composition comprises 80 mM Arginine base, 85 mM glutamic acid and 100 mM L-Proline. In various embodiments, the aqueous composition comprises 80 mM Arginine base, 85 mM glutamic acid, 100 mM L-Proline, 0.01% (w/v) Polysorbate 80, pH 5.4.

Consistent with the foregoing, in some aspects, the composition of the present disclosure comprises about 180 mg/mL to about 210 mg/mL anti-TSLP antibody, about 0.01% (w/v)±0.005% (w/v) polysorbate 80, 10-125 mM arginine base, 25-225 mM glutamic acid and 0-250 mM proline, wherein the viscosity of the composition is less than about 22 cP (e.g., 15, 17 or 20 cP) at 23° C. and the pH is less than about 5.7, optionally, about 5.4. Optionally, the anti-TSLP antibody comprises (A) a light chain variable domain comprising: (i) a light chain CDR1 sequence comprising the amino acid sequence set forth in SEQ ID NO:3; (ii) a light chain CDR2 sequence comprising the amino acid sequence set forth in SEQ ID NO:4; and (iii) a light chain CDR3 sequence comprising the amino acid sequence set forth in SEQ ID NO:5; and (B) a heavy chain variable domain comprising: (i) a heavy chain CDR1 sequence comprising the amino acid sequence set forth in SEQ ID NO:6; (ii) a heavy chain CDR2 sequence comprising the amino acid sequence set forth in SEQ ID NO:7, and (iii) a heavy chain CDR3 sequence comprising the amino acid sequence set forth in SEQ ID NO:8. In various embodiments, the aqueous composition comprises 95 mM arginine base and 170 mM glutamic acid. In various embodiments, the aqueous composition comprises 50 mM arginine base, 95 mM glutamic acid and 85 mM L-Proline. In various embodiments, the aqueous composition comprises 95 mM arginine base, 170 mM glutamic acid, 0.01% (w/v) polysorbate 80, pH 5.4. In various embodiments, the aqueous composition comprises 50 mM arginine base, 95 mM glutamic acid and 85 mM L-Proline, 0.01% (w/v) polysorbate 80, pH 5.4.

Consistent with the foregoing, in some aspects, the composition of the present disclosure comprises about 180 mg/mL to about 210 mg/mL anti-TSLP antibody, about 0.01% (w/v)±0.005% (w/v) polysorbate 80, comprises 15-130 mM calcium, 30-300 mM glutamate and 0-250 mM proline, wherein the viscosity of the composition is less than about 20 cP (e.g., 15 or 17 cP) at 23° C. and the pH is less than about 5.5, optionally, about 5.0. Optionally, the anti-TSLP antibody comprises (A) a light chain variable domain comprising: (i) a light chain CDR1 sequence comprising the amino acid sequence set forth in SEQ ID NO:3; (ii) a light chain CDR2 sequence comprising the amino acid sequence set forth in SEQ ID NO:4; and (iii) a light chain CDR3 sequence comprising the amino acid sequence set forth in SEQ ID NO:5; and (B) a heavy chain variable domain comprising: (i) a heavy chain CDR1 sequence comprising the amino acid sequence set forth in SEQ ID NO:6; (ii) a heavy chain CDR2 sequence comprising the amino acid sequence set forth in SEQ ID NO:7, and (iii) a heavy chain CDR3 sequence comprising the amino acid sequence set forth in SEQ ID NO:8. In various embodiments, the aqueous composition comprises 100 mM calcium and 230 mM glutamate. In various embodiments, the aqueous composition comprises 60 mM calcium, 140 mM glutamate and 70 mM L-Proline. In various embodiments, the aqueous composition comprises 100 mM calcium, 230 mM glutamate, 0.01% (w/v) polysorbate 80, pH 5.0. In various embodiments, the aqueous composition comprising 60 mM calcium, 140 mM glutamate, 70 mM L-Proline, 0.01% (w/v) polysorbate 80, pH 5.0.

Consistent with the foregoing, in some aspects, the composition of the present disclosure comprises about 180 mg/mL to about 210 mg/mL anti-TSLP antibody, about 0.01% (w/v)±0.005% (w/v) polysorbate 80, 15-195 mM calcium and 25-320 mM and 0-220 mM proline, wherein the viscosity of the composition is less than about 20 cP (e.g., 15 or 17 cP) at 23° C. and the pH is less than about 5.5, optionally, about 5.0. Optionally, the anti-TSLP antibody comprises (A) a light chain variable domain comprising: (i) a light chain CDR1 sequence comprising the amino acid sequence set forth in SEQ ID NO:3; (ii) a light chain CDR2 sequence comprising the amino acid sequence set forth in SEQ ID NO:4; and (iii) a light chain CDR3 sequence comprising the amino acid sequence set forth in SEQ ID NO:5; and (B) a heavy chain variable domain comprising: (i) a heavy chain CDR1 sequence comprising the amino acid sequence set forth in SEQ ID NO:6; (ii) a heavy chain CDR2 sequence comprising the amino acid sequence set forth in SEQ ID NO:7, and (iii) a heavy chain CDR3 sequence comprising the amino acid sequence set forth in SEQ ID NO:8. In various embodiments, the aqueous composition comprises 110 mM calcium and 240 mM glutamic acid. In various embodiments, the aqueous composition comprises 70 mM calcium, 145 mM glutamate and 60 mM L-Proline. In various embodiments, the aqueous composition comprising 110 mM calcium, 240 mM glutamate, 0.01% (w/v) polysorbate 80, pH 5.0. In various embodiments, the aqueous composition comprises 70 mM calcium, 145 mM glutamate, 60 mM L-Proline, 0.01% (w/v) polysorbate 80, pH 5.0.

Methods of Use

Without being bound to a particular theory and based at least in part on the data presented herein, the presently disclosed compositions are particularly well-suited for treatment of patient suffering from an inflammatory disease. As used herein, “inflammatory disease” refers to a medical condition involving abnormal inflammation caused by the immune system attacking the body's own cells or tissues, which may result in chronic pain, redness, swelling, stiffness, and damage to normal tissues. Inflammatory diseases include, for example, asthma, chronic peptic ulcer, tuberculosis, periodontitis, sinusitis, active hepatitis, ankylosing spondylitis, rheumatoid arthritis, chronic obstructive pulmonary disease (COPD), Crohn's disease, ulcerative colitis, osteoarthritis, atherosclerosis, systemic lupus erythematosus, atopic dermatitis, eosinophilic esophagitis (EoE), nasal polyps, chronic spontaneous urticaria, Ig-driven disease (such as IgA nephropathy & lupus nephritis), eosinophilic gastritis, chronic sinusitis without nasal polyps and idiopathic pulmonary fibrosis (IPF) and the like. In exemplary aspects, the inflammatory disease is asthma, atopic dermatitis, COPD, eosinophilic esophagitis (EoE), nasal polyps and chronic spontaneous urticaria, Ig-driven disease (such as IgA nephropathy & lupus nephritis), eosinophilic gastritis, chronic sinusitis without nasal polyps and idiopathic pulmonary fibrosis (IPF). In exemplary aspects, the inflammatory disease is atopic dermatitis (AD). In various aspects, the inflammatory disease is asthma. In various aspects, the inflammatory disease is COPD.

Accordingly, use of the presently disclosed composition for treating an inflammatory disease is provided herein. In exemplary aspects, the inflammatory disease is selected from the group consisting of: asthma, atopic dermatitis, chronic obstructive pulmonary disease (COPD), eosinophilic esophagitis (EoE), nasal polyps, chronic spontaneous urticaria, Ig-driven disease (such as IgA nephropathy & lupus nephritis), eosinophilic gastritis, chronic sinusitis without nasal polyps and idiopathic pulmonary fibrosis (IPF). Optionally, the inflammatory disease is atopic dermatitis. In various aspects, the inflammatory disease is asthma. In various aspects, the inflammatory disease is COPD. Also the present disclosure provides a method for treating an inflammatory disease in a subject. In exemplary embodiments, the method comprises administering to the subject a therapeutically effective amount of the presently disclosed composition. In various aspects, the inflammatory disease is selected from the group consisting of: asthma, atopic dermatitis, chronic obstructive pulmonary disease (COPD), eosinophilic esophagitis (EoE), nasal polyps, chronic spontaneous urticaria, Ig-driven disease (such as IgA nephropathy & lupus nephritis), eosinophilic gastritis, chronic sinusitis without nasal polyps and idiopathic pulmonary fibrosis (IPF). Optionally, the inflammatory disease is atopic dermatitis. In various instances, the presently disclosed composition is administered to the subject by subcutaneous administration. In exemplary instances, about 1 mL to about 5 mL, e.g., about 1 mL to about 3 mL of the aqueous composition is administered to the subject.

Asthma

The term “asthma” as used herein refers to allergic, non-allergic, eosinophilic, and non-eosinophillic asthma.

The term “allergic asthma” as used herein refers to asthma that is triggered by one or more inhaled allergens. Such patients have a positive IgE fluorescence enzyme immunoassay (FEIA) level to one or more allergens that trigger an asthmatic response.

Typically, most allergic asthma is associated with Th2-type inflammation.

The term “non-allergic asthma” refers to patients that have low eosinophil, low Th2, or low IgE at the time of diagnosis. A patient who has “non-allergic asthma” is typically negative in the IgE fluorescence enzyme immunoassay (FEIA) in response to a panel of allergens, including region-specific allergens. In addition to low IgE, those patients often have low or no eosinophil counts and low Th2 counts at the time of diagnosis.

The term “severe asthma” as used herein refers to asthma that requires high intensity treatment (e.g., GINA Step 4 and Step 5) to maintain good control, or where good control is not achieved despite high intensity treatment (GINA, Global Strategy for Asthma Management and Prevention. Global Initiative for Asthma (GINA) December 2012).

The term “eosinophilic asthma” as used herein refers to an asthma patient having a screening blood eosinophil count of ≥250 cells/μL. “Low eosinophilic” asthma refers to asthma patients having less than 250 cells/uL blood or serum.

The term “Th2-type inflammation” as used herein refers to a subject having a screening blood eosinophil count ≥140 cells/μL and a screening total serum IgE level of >100 IU/mL (Corren et al, N Engl J Med. 22; 365(12)1088-98, 2011). A “Th2 high” asthma population or profile refers to a subject having IgE >100 IU/mL and Blood Eosinophil Count ≥140 cells/μL. A “Th2 low” asthma population refers to a subject having IgE <100 IU/mL and Blood Eosinophil Count ≤140 cells/μL

Atopic Dermatitis

In various instances, the inflammatory disease is atopic dermatitis (AD), which is a common allergic inflammatory skin disease, also known as eczema. AD is the most common skin disorder in children and is characterized by strong itchy and inflamed skin and chronic lichenified, more scaly plaques. While the cause of AD is unknown, the current theory is that AD is a condition in which the primary skill barrier is defected leading to other atopic conditions. AD is reviewed in Kapur et al., Atopic dermatitis. Allergy Asthma Clin Immunol 14, 52 (2018) doi:10.1186/s13223-018-0281-6. AD, like asthma and allergic rhinitis, involve a T helper type 2 (Th2) cell-mediated allergic inflammation caused by a secretion of IL-4, IL-5, IL-13 and TNFα by CD4+ T-cells. These cytokines cause increase IgE antibody production by B-cells and IgG binds to mast cells, facilitating the initiation of allergic reactions and driving of leukocytes into the skin dermis. Indra, Exper Rev Proteomics 10(4): 309-311 (2013). In exemplary aspects, the AD is characterized by higher expression of TSLP. In various aspects, the AD is characterized by TSLP secretion by epidermal keratinocytes which triggers TH2 cytokine associated inflammation.

In exemplary aspects, the AD is chronic and may flare periodically. In various aspects, the patient experiences one or more of the following symptoms of AD:
dry skin, itching, which can be severe, especially at night,
red to brown or gray colored patches (e.g., located on the hands, feet, ankles, wrists, neck, upper chest, eyelids, inside the elbow or knee, face or scalp),
small, raised bumps,
thickened, cracked scaly skin, and
raw, sensitive swollen skin from scratching.
   (From mayoclinic.org/diseases-conditions/atopic-dermatitis-eczema/symptoms-causes/syc-20353273)

In various aspects, the AD exists with one or more of asthma, hay fever, chronic itchy, scaly skin, skin infection, irritant hand dermatitis, allergic rhinitis, allergic contact dermatitis, or sleep problems.

In various embodiments, use of the presently disclosed composition eliminates the need for corticosteroid therapy or other medication used to treat AD.

In alternative embodiments, the methods comprises administering the presently disclosed composition in combination with another anti-inflammatory or anti-AD treatment. For instance, in various aspects, the method further comprises administering a corticosteroid (e.g., prednisone) or calcineurin inhibitor (e.g., tacrolimus, pimecrolimus), an antibiotic or a biologic (e.g., dupilumab). In exemplary aspects, the method further comprises light therapy treatment, e.g., phototherapy with sunlight, UVA or UVB.

Subjects

It is contemplated that the subject is human. The subject may be an adult, an adolescent or a child.

In various aspects, the subject shows signs or symptoms of the inflammatory disease, e.g., AD or COPD, such as any one or more of those described above. In various aspects, the subject also suffers from one or more of asthma, hay fever, chronic itchy, scaly skin, skin infection, irritant hand dermatitis, allergic contact dermatitis, or sleep problems. In various aspects, the subject has previously been treated or is currently being treated with an anti-AD or anti-inflammatory treatment, e.g., a corticosteroid (e.g., prednisone) or calcineurin inhibitor (e.g., tacrolimus, pimecrolimus), an antibiotic or a biologic (e.g., dupilumab). In exemplary aspects, the method further comprises light therapy treatment, e.g., phototherapy with sunlight, UVA or UVB. Optionally, the subject has never been treated with any of: a corticosteroid (e.g., prednisone) or calcineurin inhibitor (e.g., tacrolimus, pimecrolimus), an antibiotic or a biologic (e.g., dupilumab). In exemplary aspects, the method further comprises light therapy treatment, e.g., phototherapy with sunlight, UVA or UVB.

Therapeutic Regimens, Dosages, and Routes of Administration

Therapeutic antibody (or antibody variant) compositions may be delivered to the patient at multiple sites. The multiple administrations may be rendered simultaneously or may be administered over a period of time. In certain cases it is beneficial to provide a continuous flow of the therapeutic composition. Additional therapy may be administered on a period basis, for example, hourly, daily, weekly, every 2 weeks, every 3 weeks, monthly, or at a longer interval.

In various embodiments, the amounts of therapeutic agent, such as a bivalent antibody having two TSLP binding sites, in a given dosage may vary according to the size of the individual to whom the therapy is being administered as well as the characteristics of the disorder being treated.

In exemplary treatments, the composition provides a dose of the anti-TSLP antibody or antibody variant within the range of about 210 mg to about 420 mg per daily dose. For example, the dose provided may be about 210 mg, 280 mg or 420 mg. In various embodiments, the composition comprising the anti-TSLP antibody or antibody variant may be administered at a dose of about 210, 220, 230, 240, 250, 260, 270, 280, 290, 300, 310, 320, 330, 340, 350, 360, 370, 380, 390, 400, 410 or 420 mg per dose. These concentrations may be administered as a single dosage form or as multiple doses. The above doses are given every two weeks or every four weeks. In various embodiments, the anti-TSLP antibody or antibody variant is administered at a single dose of 280 mg or 420 mg every two weeks or every four weeks. In various embodiments, the anti-TSLP antibody or antibody variant is administered at a single dose of 210 mg every two weeks or every four weeks. In various embodiments, the composition comprising greater than about 100 mg/mL, or greater than about 140 mg/ml, anti-TSLP antibody as described herein is administered to the subject at an interval of every two weeks or every four weeks.

For antibody variants, the amount of antibody variant should be such that the number of TSLP binding sites that are in the dose have an equimolar number of TSLP binding sites to canonical bivalent antibody described above.

It is contemplated that the composition of the present disclosure comprising the anti-TSLP antibody or antibody variant is administered every 2 weeks or every 4 weeks for a period of at least 4 months, 6 months, 9 months, 1 year or more. In various embodiments, the administration is subcutaneous or intravenous.

Methods of Manufacture

Methods of making the composition of the present disclosure are further provided herein. Accordingly, methods of making a stable, liquid antibody composition having a viscosity of less than about 100 cP and comprising (A) an anti-TSLP antibody at a concentration greater than about 140 mg/mL, (B) a surfactant, and (C) a basic amino acid or a salt thereof, a calcium salt, a magnesium salt, or a combination thereof, are further provided. In exemplary embodiments, the method comprises: (i) combining the antibody with an aqueous solution comprising about 10 mM to about 200 mM or about 50 mM to about 150 mM basic amino acid or a salt thereof, a calcium salt, a magnesium salt, or a combination thereof and (ii) adding a surfactant to achieve a final concentration of about 0.01% (w/v)±0.005% (w/v) surfactant.

In exemplary aspects, the composition comprises about 160 mg/mL to about 250 mg/mL of an anti-TSLP antibody, optionally, about 180 mg/mL to about 225 mg/mL or about 180 mg/mL to about 200 mg/mL. Optionally, the composition comprises about 160 mg/mL to about 250 mg/mL of an anti-TSLP antibody, optionally, about 165 mg/mL to about 225 mg/mL or about 165 mg/mL to about 200 mg/mL. In various aspects, the composition comprises about 175 mg/mL to about 185 mg/mL of an anti-TSLP antibody, optionally, about 175 mg/mL, about 176 mg/mL, about 177 mg/mL, about 178 mg/mL, about 179 mg/mL, about 180 mg/mL, about 181 mg/mL, about 182 mg/mL, about 183 mg/mL, about 184 mg/mL, about 185 mg/mL). In various aspects, the composition comprises about 180 mg/mL anti-TSLP antibody. In various instances, the concentration of the anti-TSLP antibody is about 189 mg/mL or about 190 mg/mL to about 230 mg/mL or about 231 mg/mL. Optionally, the concentration of the anti-TSLP antibody is about 205 mg/mL to about 215 mg/mL, optionally, about 210 mg/mL or about 205 mg/mL, about 206 mg/mL, about 207 mg/mL, about 208 mg/mL, about 209 mg/mL, about 210 mg/mL, about 211 mg/mL, about 212 mg/mL, about 213 mg/mL, about 214 mg/mL, about 215 mg/mL. In exemplary aspects, the aqueous solution comprises an organic salt of arginine, lysine, or histidine. In exemplary aspects, the aqueous solution comprises arginine acetate, arginine aspartate, arginine glutamate, arginine glycolate, arginine lactate, arginine methanesulfonate, arginine propionate, histidine acetate, histidine aspartate, histidine glutamate, histidine glycolate, histidine lactate, histidine methanesulfonate, histidine propionate, lysine acetate, lysine aspartate, lysine glutamate, lysine glycolate, lysine lactate, lysine methanesulfonate, lysine propionate, calcium acetate, calcium aspartate, calcium glutamate, calcium glycolate, calcium lactate, calcium methanesulfonate, calcium propionate, magnesium acetate, magnesium aspartate, magnesium glutamate, magnesium glycolate, magnesium lactate, magnesium methanesulfonate, magnesium propionate, or a combination thereof. In various aspects, the aqueous solution comprises about 15 mM to about 200 mM or about 50 mM to about 150 mM salt (basic amino acid salt, calcium salt, magnesium salt). In some aspects, the surfactant is polysorbate 80 or polysorbate 20. In exemplary instances, the surfactant is polysorbate 80 and the final concentration of PS80 is about 0.01% (w/v). In exemplary embodiments, the anti-TSLP antibody is tezepelumab.

Kits

The present disclosure also provides a kit including a composition described herein together with a package insert, package label, instructions, or other labeling directing or disclosing any of the methods or embodiments disclosed herein. In certain embodiments, the present disclosure provides kits for producing a single-dose administration unit. In certain embodiments of this disclosure, kits containing single and multi-chambered pre-filled syringes (e.g., liquid syringes) are included.

The following examples are given merely to illustrate the present invention and not in any way to limit its scope.

### EXAMPLES

Throughout the examples presented herein, the following abbreviations are used: DF, diafiltration; PS80, polysorbate 80; SEC, size exclusion chromatography, F#, formulation number. Additionally, throughout these examples, the composition of the DF buffer used to make the final formulation comprising tezepelumab, as well as estimated concentrations of the components of the final formulation, are provided.

The final concentrations of certain components of the final formulations analyzed (e.g., for stability, viscosity, optionally, after storage) differ from the concentrations of the DF or dialysis buffer, depending on the presence or absence of a counterion. Without a counterion, formulations have low ionic strength. In such instances, acetate co-concentrates with tezepelumab, such that final formulations comprise a higher concentration of acetate, relative to the concentration of the DF or dialysis buffer. For example, use of a DF buffer comprising 10 mM acetate leads to about 20 mM to about 22 mM acetate in a formulation (pH 5.2) comprising 110 mg/mL tezepelumab when neither the DF buffer nor the final formulation comprises a salt (e.g., Arginine HCl) and thus is of low ionic strength. Similarly, a DF buffer comprising 10 mM acetate leads to about 23 mM to about 25 mM acetate a formulation (pH 5.2) comprising 140 mg/mL tezepelumab, without a salt (e.g., Arginine HCl). When a salt (e.g., Arginine HCl) is present, acetate does not co-concentrate with tezepelumab, and therefore the acetate concentration of the DF buffer and the acetate concentration of the final composition are generally equivalent. Additionally, excipients can be volumetrically excluded, or may be impacted by non-specific interactions. For instance, in a 110 mg/mL tezepelumab formulation, the proline concentration may be up to about 16.67% lower than what is indicated in the DF buffer, and in a 140 mg/mL tezepelumab formulation, the proline concentration may be up to about 10% to about 13.3% lower than what is indicated in the DF buffer. In view of the foregoing, throughout the following examples concentrations of the components of the final formulations are provided, taking into consideration the above described excipient exclusion and acetate co-concentration effects.

### Example 1

This example demonstrates an exemplary method of producing a high concentration tezepelumab formulation

A series of studies were carried out to develop a formulation containing a high concentration of tezepelumab (e.g., >70 mg/mL). Because the formulation was intended for subcutaneous administration, the formulation needed to be isotonic and demonstrate a viscosity suitable for this route of administration. For example, a formulation of tezepelumab (110 mg/mL) needed to have a viscosity of about 15 cP at 23° C. It was also desirable for the formulation to be stable following storage for more than 1 year (e.g., at least 2 or 3 years) at 2° C. to 8° C.

To prepare high concentration formulations of tezepelumab, an initial solution containing tezepelumab (70 mg/mL) in acetate (pH 5.2) was dialyzed against a diafiltration (DF) buffer. A total of 10 buffer changes was used to achieve complete buffer exchange. Using a centrifuge-concentrator, the buffer-exchanged tezepelumab solution was over-concentrated to a tezepelumab concentration that was ˜110% of the target tezepelumab concentration. For example, the buffer-exchanged tezepelumab solution was over-concentrated to ˜200 mg/mL in order to achieve a target tezepelumab concentration of 180 mg/mL. The over-concentrated solutions were diluted to the target tezepelumab concentration using the same DF buffer used in the buffer exchange step.

Following the above procedures, a series of tezepelumab formulations having varied tezepelumab concentrations (ranging from ˜150 mg/mL to about 250 mg/mL) were made for a viscosity study. Two DF buffers were used in making these formulations: a first DF buffer comprising an arginine salt (arginine glutamate) and a second DF buffer comprising proline. The arginine was present in the DF buffer at 150 mM and the proline was present in the DF buffer at 3% (w/v). Samples of each formulation were tested for viscosity using a rotational viscometer at 23° C. Reported viscosity values are at a shear rate of 1000 s⁻¹.

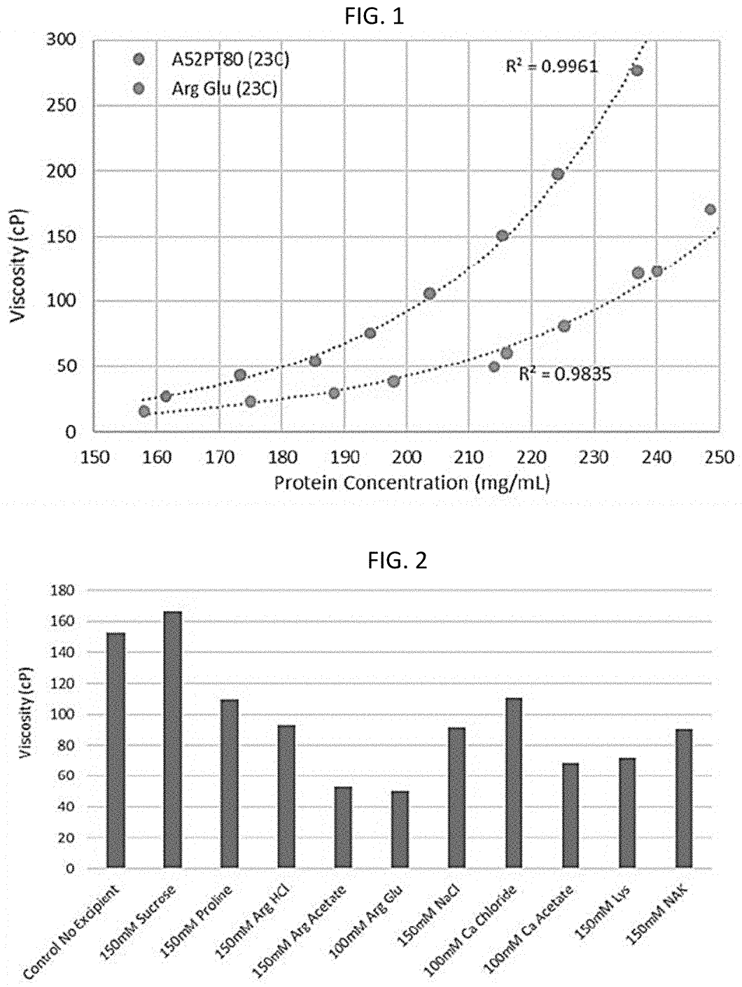

FIG. 1 provides the graphical results of this assay. FIG. 1 is a graph which plots the viscosity of each formulation as a function of tezepelumab concentration. As shown in this figure, the viscosity increases as the tezepelumab concentration increases, regardless of DF buffer used. The viscosities were overall lower for the formulations made using the first DF buffer, supporting the use of an arginine salt in low viscosity formulations of tezepelumab.

Since lysine is structurally similar to arginine inasmuch as both are basic amino acids, lysine glutamate was tested as an excipient for lowering viscosity and compared to a formulation comprising arginine glutamate. Each formulation comprised ˜195 mg/mL tezepelumab and the DF buffer used to make the final formulation comprised 100 mM arginine glutamate or 100 mM lysine glutamate. Samples of each formulation were tested for viscosity using a rotational viscometer at 23° C. Reported viscosity values are at a shear rate of 1000 s⁻¹. The viscosity of the formulation comprising lysine glutamate was 48.9 cP, while the viscosity of the formulation comprising arginine glutamate was 35.3 cP. These data suggested that both basic amino acids, or a salt thereof, performed well to reduce the viscosity of formulations comprising high concentrations of tezepelumab.

### Example 2

This example demonstrates the viscosity-lowering effects of different excipients for tezepelumab formulations.

As arginine and lysine worked well to reduce the viscosity of formulations comprising high concentrations of tezepelumab, various arginine salts and a lysine analog were used to spike tezepelumab samples and then these samples were tested for viscosity. The arginine salts used in this study were arginine hydrochloride, arginine acetate, arginine glutamate. The lysine analog, N-acetyl lysine (NAK), was tested alongside a lysine-containing sample. Also, the effect on viscosity of calcium salts and a sodium salt was tested. Calcium chloride, calcium acetate, and sodium chloride, in particular, were used.

A sample comprising one of the above excipients was generated by precisely spiking a concentrated (5× or 10×) stock solution comprising one of the excipients into an aliquot of a concentrated tezepelumab solution. The concentrated tezepelumab solution was made by dialyzing an aqueous solution comprising 110 mg/mL tezepelumab against 10 mM Sodium Acetate, pH 4.4, using dialysis tubing having a 10,000 molecular weight cut-off, and then concentrating the dialyzed solution using Amicon Ultra centrifugal concentrators to provide an aqueous composition comprising 230 mg/mL tezepelumab. This method created sample sets with matching concentrations.

Prior to testing for viscosity, the concentration of each sample was confirmed by UV absorbance slope spectroscopy using Solo-VPE (C-Technologies) following a 5-fold gravimetric dilution. Each sample was determined to have a tezepelumab concentration of about 210 mg/mL. The concentration of each excipient in the sample was about 100 mM or about 150 mM. FIG. 2 indicates the concentration of each excipient. The final pH of samples was determined using a Seven Easy pH meter (Mettler Toledo).

Each sample was tested for viscosity as essentially described in Example 1 and compared to a control sample comprising no excipients or comprising proline or sucrose.

The results are shown in FIG. 2. As shown in FIG. 2, all samples comprising an arginine salt, lysine or NAK exhibited lower viscosities than controls. Though the calcium acetate-containing sample demonstrated a lower viscosity, relative to controls, the calcium chloride-containing sample exhibited a viscosity that was the same as one of the controls (proline control). Only slightly better in viscosity-reducing performance was sodium chloride and arginine HCl. While the samples with a chloride-containing excipient exhibited a viscosity lower than the controls, these samples did not perform as well as the excipients which did not include chloride.

### Example 3

This example demonstrates the viscosity-reducing effects of various excipients on high concentration tezelpelumab formulations.

An additional study was carried out to test the viscosity-lowering effects of additional arginine salts. In this study, each sample was determined to have a tezepelumab concentration of about 190 mg/mL and the samples comprised 60 mM of one of the following excipients: arginine hydrochloride, arginine acetate, arginine glutamate, arginine propionate, arginine aspartate, arginine methanesulfonate, arginine glycolate, or arginine phosphate. In this study, a control sample comprising no excipients was made and tested. The samples were generated and tested for viscosity as essentially described in Examples 1 and 2.

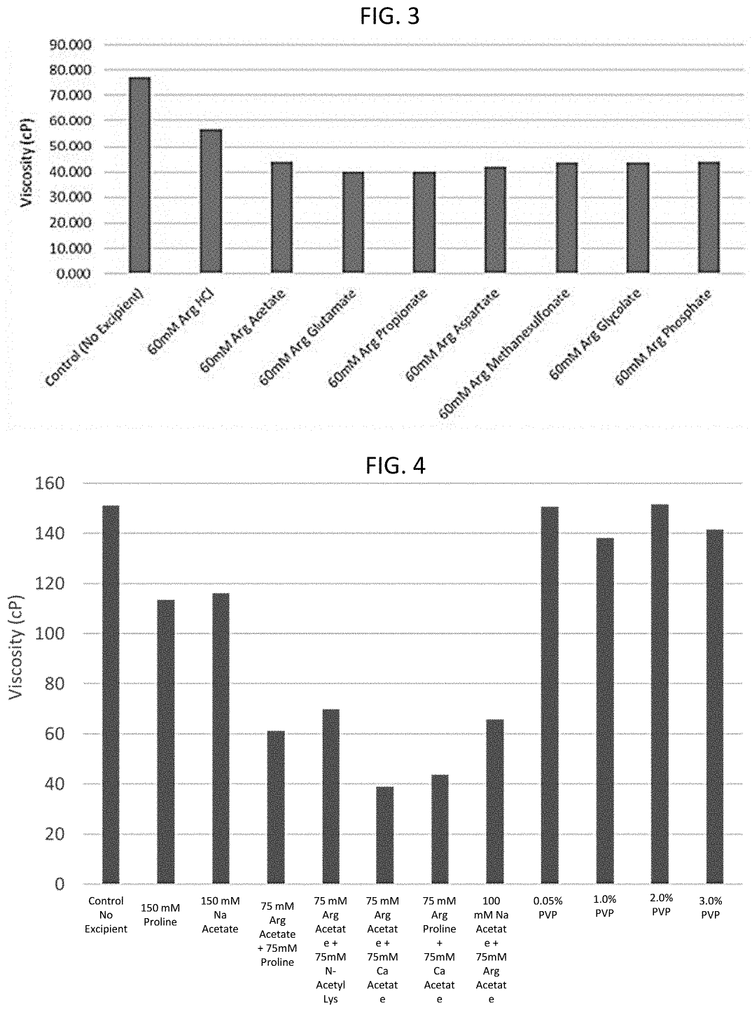

The results are provided in FIG. 3. As shown in FIG. 3, all samples comprising an arginine salt exhibited a lower viscosity relative to the control. Of the arginine-salt containing samples, the sample comprising arginine hydrochloride exhibited the highest viscosity, consistent with the data of FIG. 2, which showed that samples comprising chloride-containing excipients do not generally perform well as a viscosity-lowering excipient.

### Example 4

This example demonstrates the effects of combined excipients on viscosity.

Since arginine acetate and calcium acetate worked well to reduce the viscosity of high concentration tezepelumab samples (FIG. 2), the effect of combining these excipients with each other or with another excipient was assayed. Each of NAK, sodium acetate, and proline were combined with arginine acetate in three different tezepelumab samples, and in yet another sample, calcium acetate was combined with proline. As a control, a sample comprising no excipient or a sample comprising sodium acetate, or proline were used. Additionally, in this study, polyvinylpyrrolidone (PVP) was tested in varied amounts ranging from 0.5% (w/v) to 3% (w/v). Each sample comprised ˜210 mg/mL tezepelumab and was made following the procedure described in Example 2. A summary of the samples are described in Table 1.

TABLE 1

| Sample# | Excipients in Sample |
|---|---|
| 1 | No excipients - Control |
| 2 | Proline (150 mM) |
| 3 | Na Acetate (150 mM) |
| 4 | Arg Acetate (75 mM) + Proline (75 mM) |
| 5 | Arg Acetate (75 mM) + NAK (75 mM) |
| 6 | Arg Acetate (75 mM) + Ca Acetate (75 mM) |
| 7 | Arg Acetate (75 mM) + Na Acetate (100 mM) |
| 8 | PVP (0.5% (w/v)) |
| 9 | PVP (1.0% (w/v)) |
| 10 | PVP (2.0% (w/v)) |
| 11 | PVP (3.0% (w/v)) |

Viscosity was assayed as essentially described in Example 1.

The results are shown in FIG. 4. As shown in FIG. 4, combining proline or sodium acetate with arginine acetate led to a lower viscosity, relative to the sample comprising proline as the only excipient or sodium acetate as the only excipient. Combining proline with calcium acetate also led to a lowered viscosity compared to the sample comprising only proline. Sodium acetate exhibited viscosity-lowering effects, only when it was combined with arginine acetate. The sample comprising both arginine acetate and calcium acetate demonstrated the lowest viscosity of all the samples tested in this study. PVP at any of the tested concentrations did not work at all to reduce viscosity of the tezepelumab samples.

An additional study directed to testing an arginine salt combined with a second excipient was carried out. In this study, glycine and tris acetate were tested with or without arginine acetate. As a control, a sample comprising no excipient or a sample comprising proline or tris hydrochloride were used in this study. Additionally, samples comprising other arginine salts, arginine propionate, and arginine methanesulfonate were tested. Furthermore, a sample comprising tris hydrochloride was made and tested. Each sample comprised ˜210 mg/mL tezepelumab and were made following the procedure described in Example 2. A summary of the excipients and their concentration in each sample is provided in Table 2.

TABLE 2

| Sample# | Excipients in sample |
|---|---|
| 12 | No excipients - Control |
| 13 | Proline (150 mM) |
| 14 | Glycine (150 mM) |
| 15 | Tris HCl (150 mM) |
| 16 | Tris acetate (150 mM) |
| 17 | Arginine propionate (150 mM) |
| 18 | Arginine aspartate (150 mM) |
| 19 | Arginine methanesulfonate (150 mM) |
| 20 | Arginine aspartate (75 mM) + Tris acetate (75 mM) |
| 21 | Arginine aspartate (75 mM) + Glycine (75 mM) |

The formulations were tested for viscosity and compared to a control formulation comprising no excipients or comprising 150 mM proline. Viscosity was assayed as essentially described in Example 1.

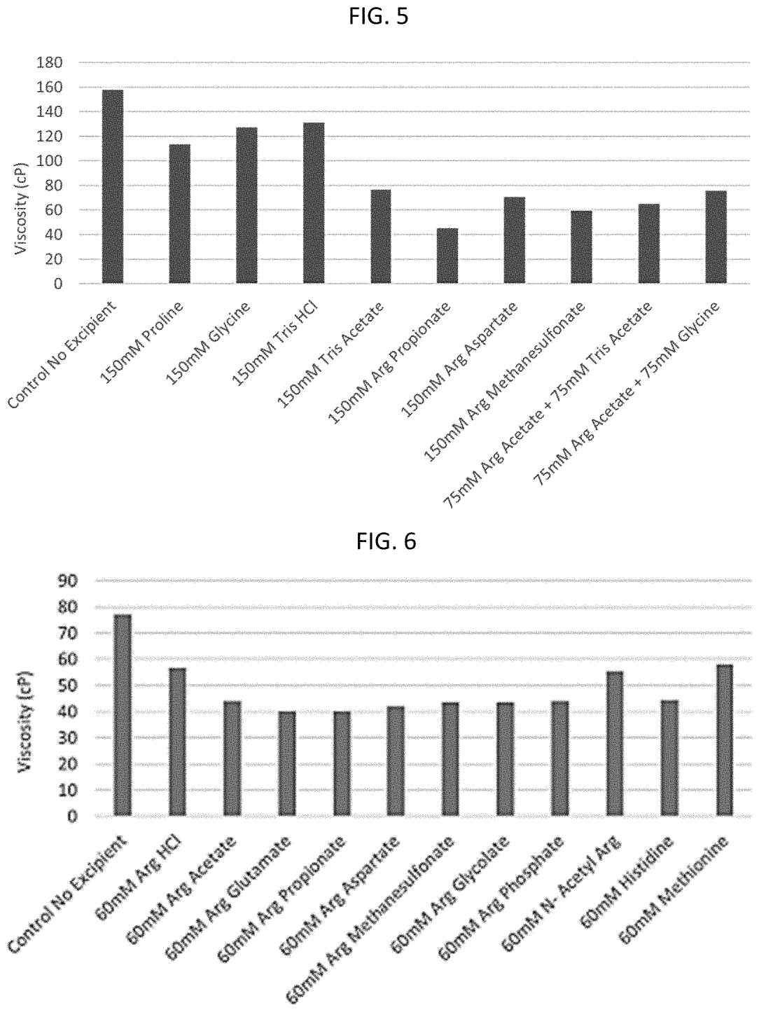

The viscosity results of each formulation are provided in FIG. 5. As shown in the figure, combining arginine acetate with either tris acetate or glycine led to lower viscosities relative to the samples comprising tris acetate alone or glycine alone. Tris hydrochloride did not perform well as a viscosity lowering agent, consistent with the prior studies which suggested that excipients comprising chloride do not perform well to reduce viscosity of high concentration tezepelumab samples.

### Example 5

This example demonstrates the viscosity-lowering effects of several excipients on samples comprising high concentrations of tezepelumab.

The basic amino acids, arginine and lysine, worked well to reduce the viscosity of formulations comprising high concentrations of tezepelumab. In one study, a sample comprising histidine was used and compared to samples comprising an arginine salt. In particular, samples comprising ˜190 mg/mL tezepelumab were made following the procedure described in Example 2. Samples comprised 60 mM histidine, or 60 mM arginine salt (arginine hydrochloride, arginine glutamate, arginine propionate, arginine aspartate, arginine methanesulfonate, arginine glycolate, arginine phosphate). Samples comprising 60 mM N-acetyl arginine (NAR) or 60 mM methionine were also made. The formulations were tested for viscosity as essentially described in Example 1 and compared to a control formulation comprising no excipients.

The results are shown in FIG. 6. As shown in FIG. 6, all arginine salts except arginine hydrochloride worked well to reduce the viscosity of the high concentration tezepelumab sample. The histidine sample worked as well as most arginine salts. NAR- and Met-containing samples worked as well as arginine hydrochloride at reducing viscosity.

### Example 6

This example demonstrates the viscosity-lowering effects on samples comprising high concentrations of tezepelumab of several combinations of excipients comprising either an arginine salt or a calcium salt.

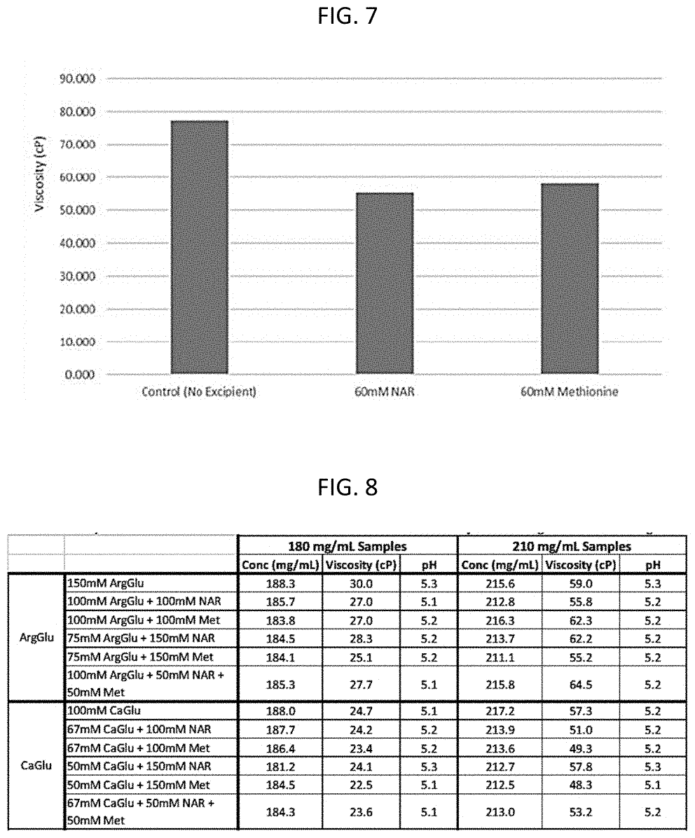

In a preliminary study, a sample comprising about 190 mg/mL tezepelumab and 60 mM NAR and a sample comprising about 190 mg/mL tezepelumab and 60 mM methionine were made. The viscosity of each sample was measured as essentially described in Example 1. As shown in FIG. 7, NAR and methionine reduced the viscosity below 60 cP.

Since arginine acetate and calcium acetate worked well to reduce the viscosity of high concentration tezepelumab formulations (FIG. 2), the effect of combining these excipients with NAR or methionine was assayed. To this end, a series of samples comprising about 180 mg/mL or about 210 mg/mL tezepelumab and arginine glutamate or calcium glutamate, alone or in combination with NAR or methionine or, in some cases, both NAR and methionine, was prepared by ultrafiltration/diafiltration (UF/DF). The pH of each sample ranged between 5.1 to 5.3. FIG. 8 provides the concentrations of each of the aforementioned excipients in each sample as well as the pH and the viscosity for each sample.

As shown in FIG. 8, the viscosities of samples comprising the lower tezepelumab concentration were lower than the viscosities of the samples comprising the higher concentration of tezepelumab. The viscosities of the lower tezepelumab concentration samples ranged from about 22 cP to about 30 cP while the viscosities of the higher tezepelumab samples ranged from about 48 cP to about 64 cP. Generally, the viscosities of the samples comprising the calcium glutamate were lower than the viscosities of the samples comprising arginine glutamate. For the lower tezepelumab concentrations, the addition of NAR, methionine or both further reduced the viscosity of the sample.

### Example 7

This example demonstrates the viscosity-lowering effects on formulations comprising high concentrations of tezepelumab of several excipients.

A sample comprising about 210 mg/mL tezepelumab and an arginine salt or another excipients known for reducing viscosity, namely, beta-alanine, sarcosine, and L-serine, was made following the procedure described in Example 2. Each sample comprised 150 mM of one of the following: beta-alanine, sarcosine, L-serine, proline, or an arginine salt (arginine propionate, arginine asparate, arginine methanesulfonate, arginine glycolate, phosphate). The samples were tested for viscosity and compared to a control sample comprising no excipient or comprising 150 mM proline. Viscosity was assayed as essentially described in Example 1.

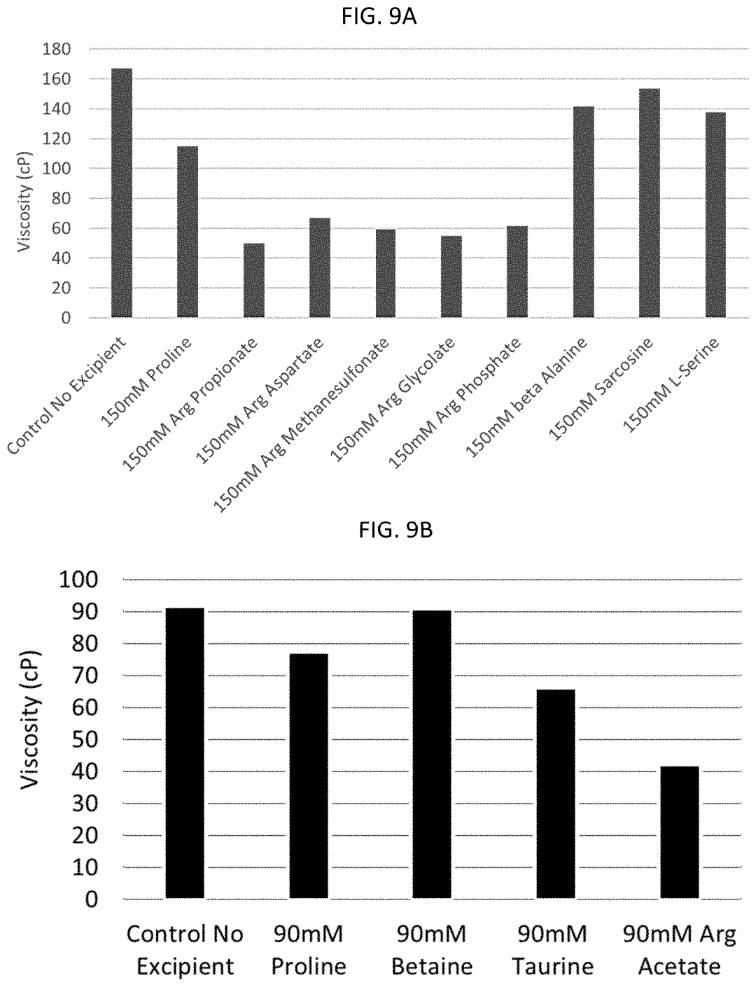

The results are shown in FIG. 9A. Consistent with prior studies, the arginine salts worked well to reduce the viscosity of samples comprising high concentrations of tezepelumab (FIG. 9A). The viscosity ranged from about 45 cP to about 65 cP. None of beta-alanine, sarcosine, and L-serine worked well to reduce the viscosity of the tezepelumab formulation (FIG. 9A).

In another study, a formulation comprising about 195 mg/mL tezepelumab and betaine, taurine, or proline was made following the procedure described in Example 2. The samples were tested for viscosity and compared to a control sample comprising no excipient. Viscosity was assayed as essentially described in Example 1.

The results are shown in FIG. 9B. Consistent with prior studies, the sample comprising the arginine salt reduced viscosity to the greatest degree. The sample comprising taurine had a modest effect on viscosity; the viscosity was slightly lower than that of the sample comprising proline (FIG. 9B). The sample comprising betaine had no effect on viscosity (FIG. 9B).

### Example 8

This example demonstrates the effect on viscosity on a high tezepelumab concentration sample of a magnesium salt.

A sample comprising about 210 mg/mL tezepelumab and 150 mM magnesium acetate was made following the procedure described in Example 2. This sample was tested for viscosity and compared to a control sample comprising no excipient or comprising 150 mM proline. Also, as a positive control, a sample comprising 150 mM arginine acetate was made and tested for viscosity. To determine if higher amounts of proline would work well to reduce the viscosity of the high tezepelumab concentration sample, a formulation comprising 300 mM proline was made and tested. Viscosity was assayed as essentially described in Example 1.

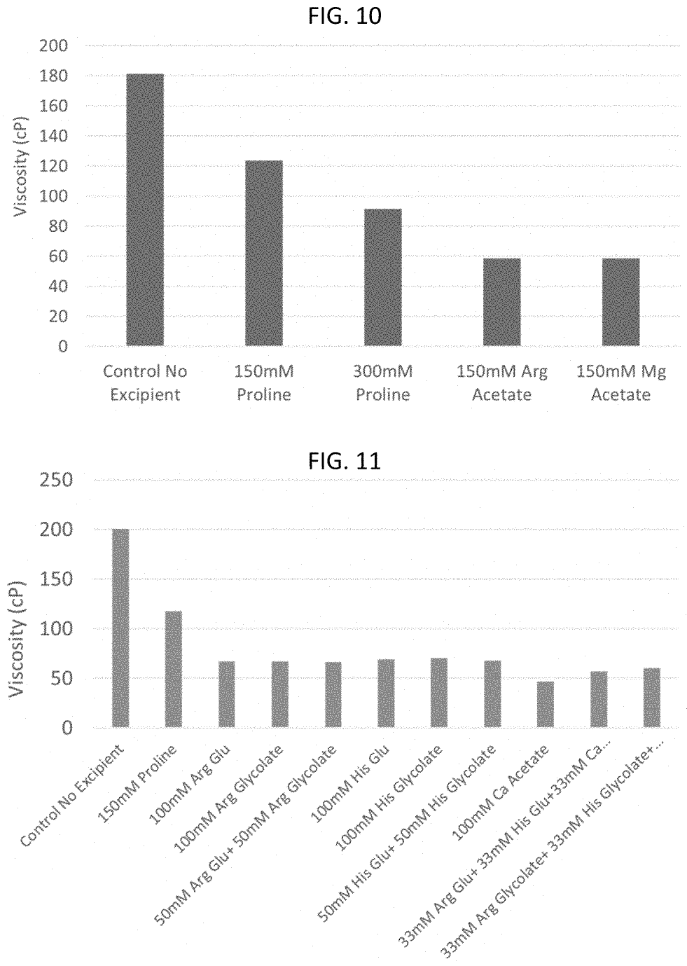

The results are shown in FIG. 10. As shown in this figure magnesium acetate worked as well as arginine acetate to lower the viscosity of the tezepelumab sample to just below 60 cP. Proline at either concentration (150 mM or 300 mM) did not reduce the viscosity of the sample as well as arginine acetate and magnesium acetate but the viscosities of both proline-containing samples were lower than the control comprising no excipients. These data suggest that magnesium salts have a viscosity-lowering effect on tezepelumab formulations.

### Example 9

This example demonstrates the effect on viscosity on a high tezepelumab concentration of histidine salts.

A sample comprising about 210 mg/mL tezepelumab and an arginine salt, a histidine salt, a calcium salt or a combination thereof was made following the procedure described in Example 2. The arginine salts used in this study were arginine glycolate and arginine glutamate. The histidine salts used in this study were histidine glycolate and histidine glutamate. The calcium salt used in this study was calcium acetate. A summary of the excipients of each sample are described in Table 3.

TABLE 3

| Sample# | Excipients in Sample |
|---|---|
| 22 | Control - no excipient |
| 23 | Proline (150 mM) |
| 24 | Arginine Glutamate (100 mM) |
| 25 | Arginine Glycolate (100 mM) |
| 26 | Arginine Glutamate (50 mM) and Arginine Glycolate |
| | (50 mM) |
| 27 | Histidine Glutamate (100 mM) |
| 28 | Histidine Glycolate (100 mM) |
| 29 | Histidine Glutamate (50 mM) and Histidine Glycolate |
| | (50 mM) |
| 30 | Calcium Acetate (100 mM) |
| 31 | Arginine Glutamate (33 mM) and Histidine Glutamate |
| | (33 mM) and Calcium Acetate (33 mM) |
| 32 | Arginine Glycolate (33 mM) and Histidine Glycolate |
| | (33 mM) and Calcium Acetate (33 mM) |

The samples were tested for viscosity and compared to a control formulation comprising no excipients or comprising 150 mM proline. Viscosity was assayed as essentially described in Example 1.

The results are shown in FIG. 11. As shown in this figure, all samples comprising an arginine salt, histidine salt, calcium salt, or a combination thereof, reduced the viscosity to below 75 cP, whereas the controls exhibited a viscosity above 100 cP.

### Example 10

This example demonstrates the effects arginine glycolate and arginine aspartate have on the viscosity of a high tezepelumab concentration formulation.

A sample comprising about 210 mg/mL tezepelumab and arginine glycolate (at a concentration of 50 mM, 75 mM, 125 mM, or 150 mM) or arginine aspartate (at a concentration of 50 mM, 75 mM, 125 mM, or 150 mM) was made following the procedure described in Example 2. The samples were tested for viscosity and compared to a control comprising no excipients or comprising 150 mM proline. Viscosity was assayed as essentially described in Example 1.

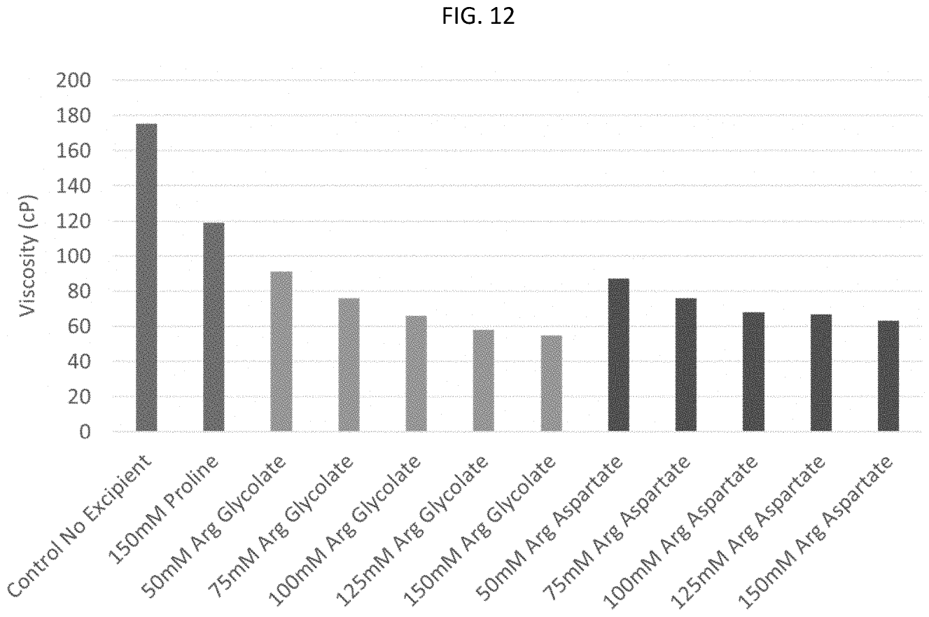

The results are shown in FIG. 12. As shown in this figure, all samples comprising an arginine salt at any concentration reduced the viscosity to below 100 cP, whereas the controls exhibited a viscosity above 110 cP. Increasing the amount of the arginine salt decreased the viscosity, though the effect was greater for arginine glycolate.

This example demonstrated the dose-viscosity-reducing effect of arginine salts.

### Example 11

This example demonstrates the effect of pH on viscosity of a high tezepelumab concentration formulation.

A sample comprising about 210 mg/mL tezepelumab and 150 mM arginine aspartate or 150 mM histidine acetate was made following the procedure described in Example 2. For the arginine aspartate samples, the pH varied from 4.75 to 5.7, and for the histidine acetate samples, the pH varied from 5.5 to 6.5. The samples were tested for viscosity and compared to a control comprising no excipients or comprising 150 mM proline. Viscosity was assayed as essentially described in Example 1.

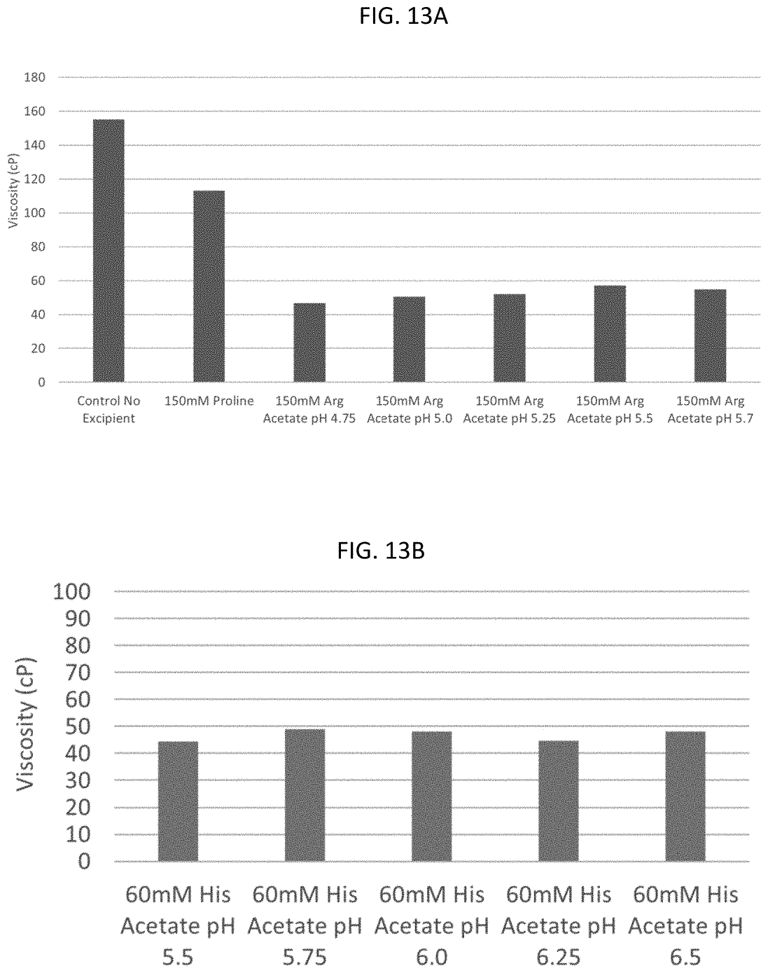

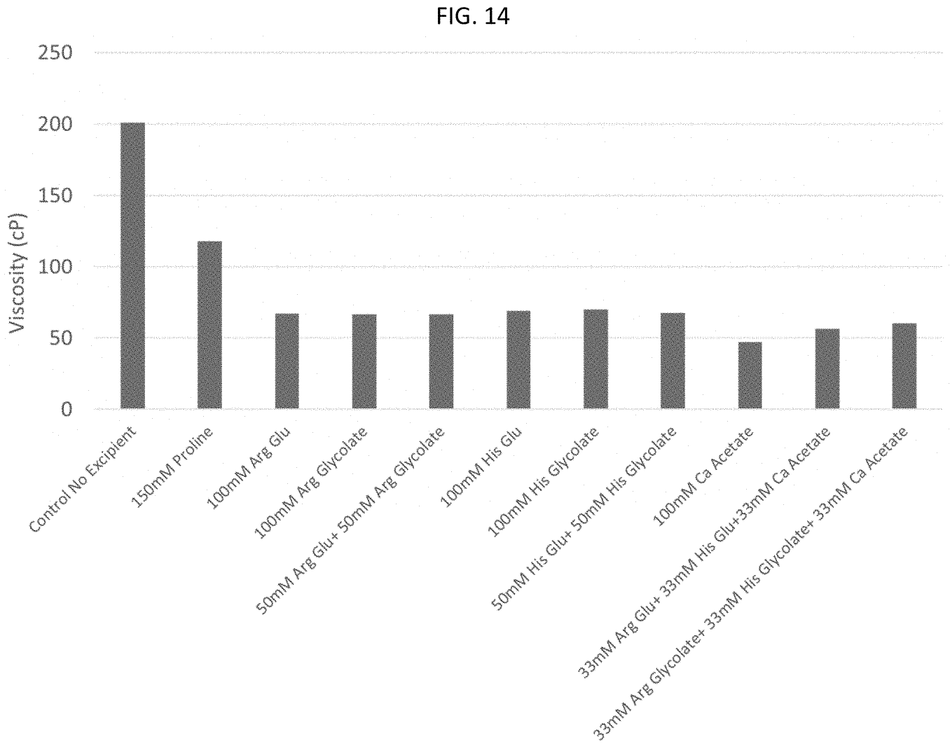

The results are shown in FIGS. 13A and 13B. As shown in these figures, the salts reduced the viscosity to below 50 cP (for the histidine salts) or below 60 cP (for the arginine salts) and the varied pH did not negatively impact the viscosity-reducing effect.

These data support that formulations comprising a high concentration of tezepelumab and an arginine salt or histidine salt and having a pH of about 4.75 to about 6.5 exhibit a reduced viscosity.

### Example 12

This example demonstrates the stability of tezepelumab formulations comprising an arginine salt following storage at 40° C. for 1 week.

A series of samples each comprising about 195 mg/mL tezepelumab and 150 mM arginine salt was made following the procedure described in Example 2. The samples were tested for stability and compared to a control comprising no excipients or comprising 150 mM proline. For stability testing, samples were filled into containers and then stored for 1 week at 40° C. Samples were tested via size exclusion chromatography (SEC) to determine the stability of the sample at various storage time points. Percentage of the high molecular weight (HMW) species for samples was reported and reflected the amount of HMW species that formed after the storage period. The results of the stability assay are shown below in Table 4. A percent high molecular weight (HMW) species is shown for each sample. The lower the % HMW indicated higher stability for the formulation.

TABLE 4

| Excipient | % HMW |
|---|---|
| Control (No excipient) | 3.42 |
| Proline | 3.25 |
| Arg HCl | 10.61 |
| Arg Acetate | 7.93 |
| Arg Glutamate | 4.37 |
| Arg Glycolate | 6.39 |
| Arg Propionate | 16.55 |
| Arg Methanesulfonate | 8.33 |
| Arg Aspartate | 4.47 |
| Arg Phosphate | 3.14 |

These data suggest that counterion identity impacts HMW formation and therefore tezepelumab stability.

### Example 13

This example demonstrates the viscosity and stability of exemplary presently disclosed tezepelumab formulations after storage at −30° C., 5° C. and 25° C. for up to 6 months.

To study the effects on viscosity and stability upon storage at different temperatures and different times of high tezepelumab concentration formulations, as well as evaluating the robustness of the formulation when subjected to drug product manufacturing process and associated process stresses, four formulations comprising a basic amino acid, or salt thereof, a calcium salt or magnesium salt and ˜180 mg/mL tezepelumab were made and then stored and/or subjected to one or more drug product manufacturing process or process-associated stresses, including stresses from one or more freeze/thaw cycles, formulation pool/mix, bioburden reduction filtration, drug product hold, sterile filtration, filling, inspection, simulated labeling/packaging, and simulated transportation.

For each formulation, an initial solution comprising tezepelumab was subject to UF/DF using a unique diafiltration (DF) buffer. After UF/DF, a surfactant was added. The components of each DF buffer used to arrive at the four formulations, as well as the amount of surfactant added post-UF/DF, are set out in Table 5. As a control, a fifth formulation (A5) was made using a DF buffer comprising 10 mM acetate, 261 mM (3.0% (w/v)) L-proline, pH 5.2, and surfactant 0.010% (w/v) polysorbate 80 (PS 80) was added after UF/DF.

TABLE 5

| | | Basic Amino Acid | | | |
|---|---|---|---|---|---|
| | | or salt thereof, | | | |
| Formu- | Tezepelumab | calcium salt or | Additional | Surfac- | Final |
| lation | (mg/mL) | magnesium salt | Excipient | tant | pH |
| A1 | 180 | 120 mM | None | 0.01% | 5.0 |
| | | calcium | | (w/v) | |
| | | glutamate | | PS80 | |
| A2 | 180 | 155 mM | None | 0.01% | 5.4 |
| | | arginine | | (w/v) | |
| | | glutamate | | PS80 | |
| A3 | 180 | 60 mM | 140 mM | 0.01% | 5.0 |
| | | calcium | proline | (w/v) | |
| | | glutamate | | PS80 | |
| A4 | 180 | 80 mM | 100 mM | 0.01% | 5.4 |
| | | arginine | proline | (w/v) | |
| | | glutamate | | PS80 | |
| A5 | 180 | n/a | 10 mM | 0.01% | 5.2 |
| | | | Acetate | (w/v) | |
| | | | 261 mM | PS80 | |
| | | | Proline | | |

Calcium glutamate was made by combining calcium hydroxide with glutamate and arginine glutamate was made by combining arginine base with glutamic acid (glutamate). Final pH was achieved by titrating with glutamic acid and determined using a Seven Easy pH meter (Mettler Toledo). The tezepelumab concentration of each formulation is confirmed by UV absorbance slope spectroscopy using Solo-VPE (C-Technologies) following a 5-fold gravimetric dilution.

The final concentrations of certain components of the final formulations analyzed (e.g., for stability, viscosity, optionally, after storage) differ from the concentrations of the DF buffer. In such instances, components, such as acetate, glutamate, and calcium co-concentrate with tezepelumab, such that final formulations comprise a higher concentration of the component (e.g., acetate, glutamate, calcium) relative to the concentration of the component in the DF buffer. For example, the concentration of glutamate in the DF buffer increases up to 15% in the final formulation comprising 180 mg/mL tezepelumab and the concentration of calcium in the DF buffer increases up to 50% in the final formulation comprising 180 mg/mL tezepelumab.

Additionally, excipients can be volumetrically excluded, or may be impacted by non-specific interactions. For instance, in a 110 mg/mL tezepelumab formulation, the proline concentration may be up to about 16.67% lower than what is indicated in the DF buffer, and in a 140 mg/mL tezepelumab formulation, the proline concentration may be up to about 10% to about 13.3% lower than what is indicated in the DF buffer. Also, for instance, in a 180 mg/mL tezepelumab formulation, the proline concentration may be about 15% lower than what is indicated in the DF buffer and the arginine concentration may be about 40-45% lower than what is indicated in the DF buffer.

The formulations in Table 5 were tested for viscosity, stability upon storage by a range of assays used to assess product quality, or a combination thereof. Viscosity was measured using an AR-G2 cone and plate rheometer (TA instruments) at a shear rate of 1000 1/sec at 23° C. Unless noted otherwise, viscosity was measured in the absence of a surfactant. For stability, samples of each formulation were filled into containers and then stored for up to 6 months (e.g., 1 week, 2 weeks, 4 weeks, 3 months, 6 months) at a temperature of about −30° C. to about 40° C. (e.g., −30° C., 5° C., 25° C., 40° C.). Samples were tested via size exclusion chromatography (SEC) to determine the stability of the formulation at various storage time points. Percentage of high molecular weight (HMW) species and percentage of the main peak for each formulation was reported. The main peak percentage reflected the amount of tezepelumab (in monomer form) that remained after the indicated storage period.

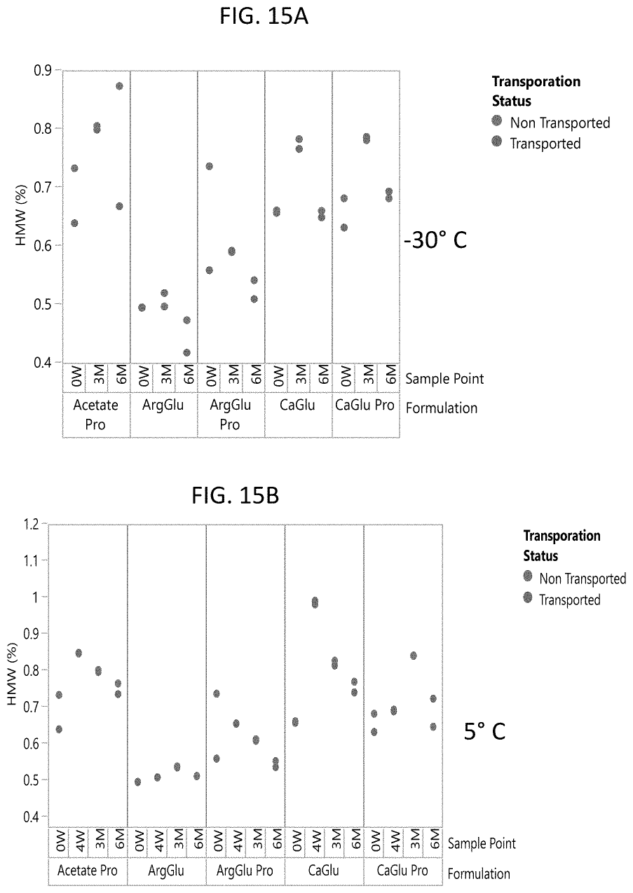

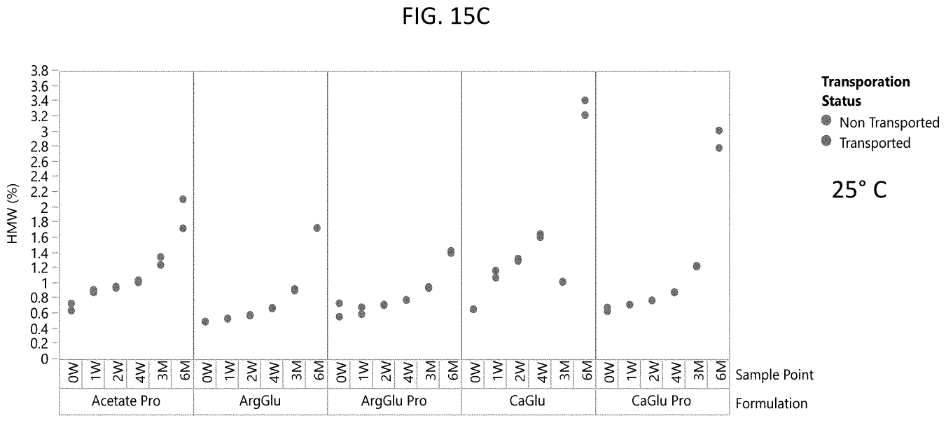

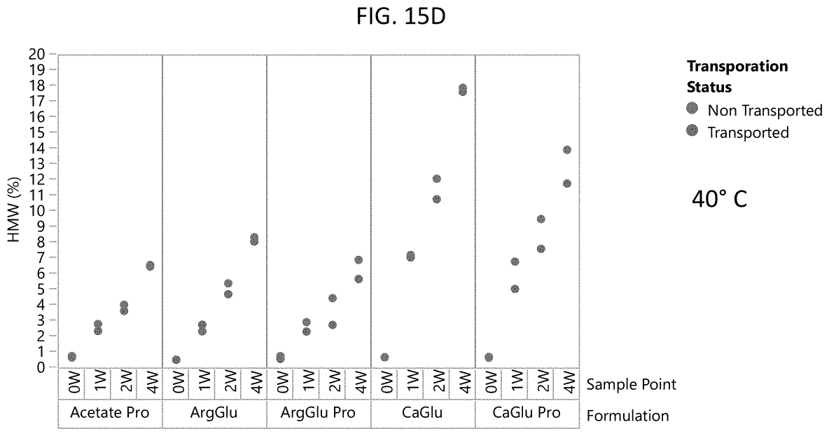

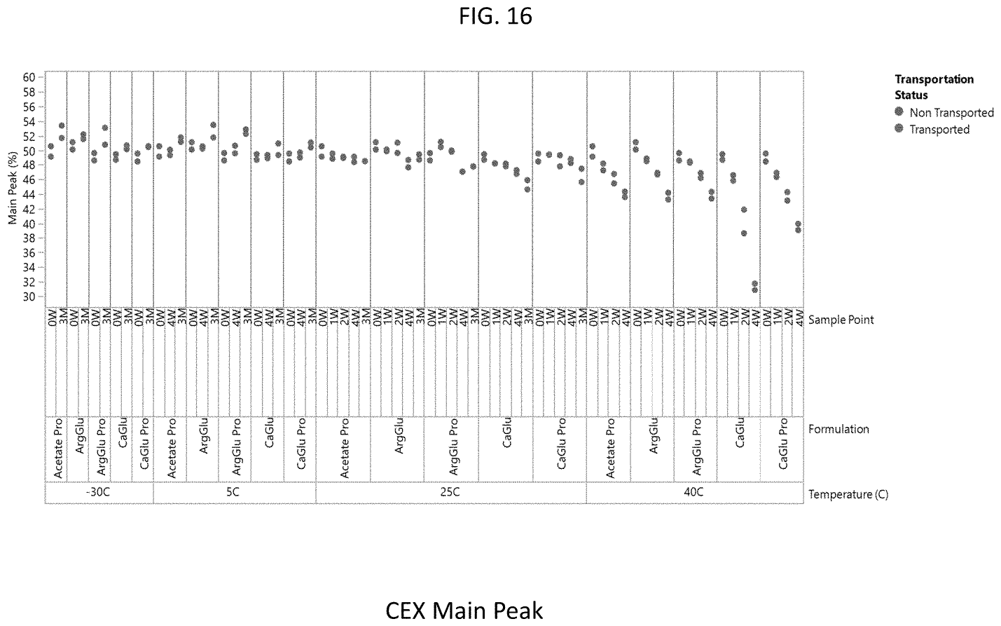

As shown in FIGS. 15A-15D and FIG. 16, arginine glutamate (ArgGlu) and arginine glutamate proline (ArgGluPro) formulations provide the best stability while maintaining a reduced viscosity. Studies out to 6 months, carried out at temperatures of −30° C., 5° C. and 25° C., confirmed that ArgGlu and ArgGluPro formulations exhibited the least protein aggregation. The ArgGlu and ArgGluPro formulations were also more stable than calcium glutamate (CaGlu) or calcium glutamate proline (CaGluPro) formulations under high stress, 40° C. after 4 weeks.

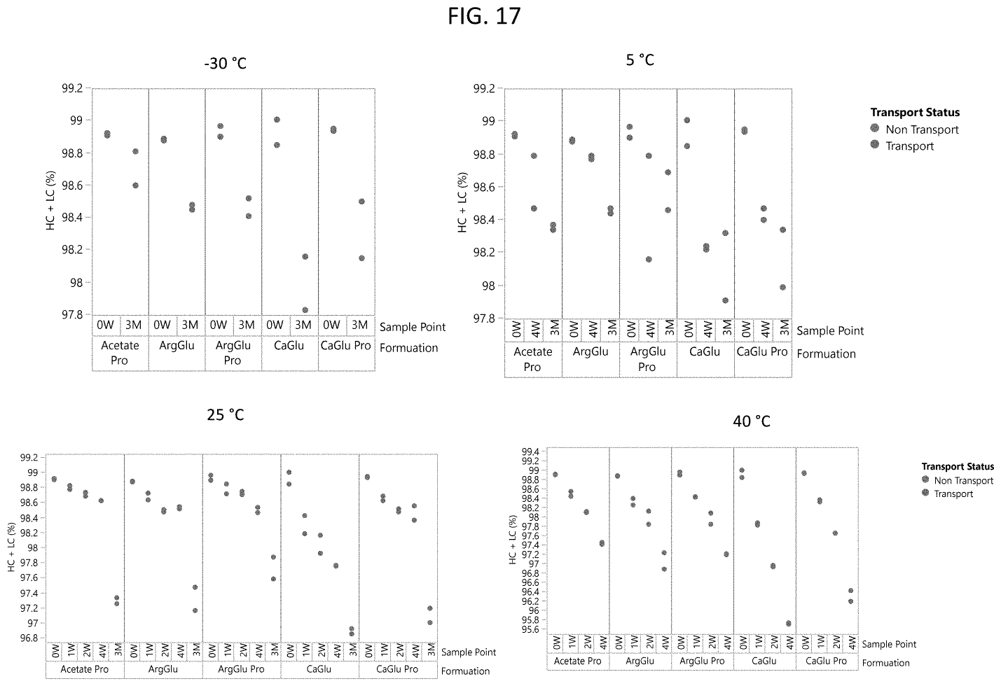

Capillary electrophoresis-sodium dodecyl sulfate (CE-SDS) is used for separation of denatured protein size variants under non-reduced (nr) or reduced conditions (RCE-SDS). Under RCE-SDS, heavy chain and light chain release and stability were measured. Heavy Chain+Light Chain release was 98%, while Heavy Chain+Light Chain stability 96% (FIG. 17) at the conditions tested.

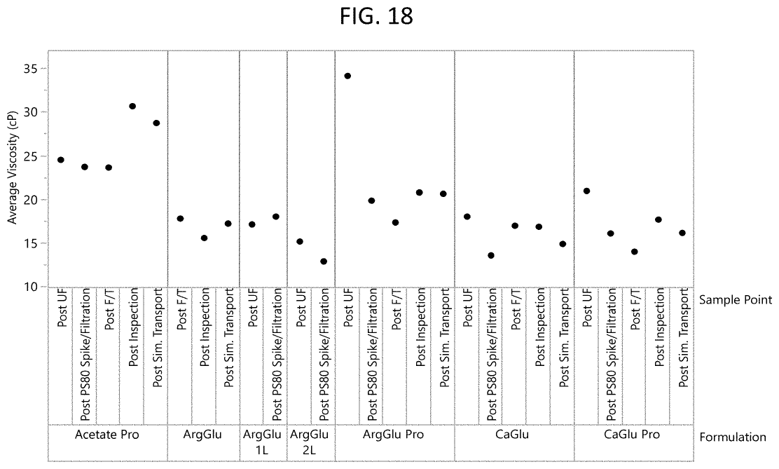

Viscosity analysis showed that ArgGlu and ArgGluPro formulations maintained viscosity below 25 cP, and at approximately 20-22 cP or lower (FIG. 18).

Analysis of stability at the six month timepoint at temperatures of −30° C., 5° C. and 25° C. confirmed that ArgGlu and ArgGluPro formulations exhibited the least protein aggregation. Overall, the conditions show that arginine glutamate and arginine glutamate proline formulations are effective in both reducing viscosity and protein aggregation in anti-TSLP antibody formulations.

Based on the results, exemplary contemplated excipient ranges are set out in Table 6. Surfactant amounts are contemplated as those in Table 5. “Formulated with” refers to amounts of excipients in the diafiltration (DF) buffer. “Formulated in” refers to the final estimated concentrations of excipients after admixture and taking into account any exclusion properties or co-concentration effects resulting from the mixture.

TABLE 6

| | | | Exemplary DF | Exemplary | | | Exemplary |
|---|---|---|---|---|---|---|---|
| | | | Buffer | DF Buffer | | Exemplary Drug | Drug Product |
| | | DF Buffer | Concentration | Concentration | Drug Product | Product | Concentration |
| | | Concentration | Target (mM) | Target | Concentration | Concentration | Target |
| | | Effective | without | (mM) | Effective | Target (mM) | (mM) |
| | | Range (mM) | Proline | with Proline | Range (mM) | without Proline | with Proline |
| | | Formulated With | | | Formulated In | | |
| Arginine | Arginine | 25-190 | 140 | 80 | 10-125 | 95 | 50 |
| Glutamate | Glutamate | 25-200 | 150 | 85 | 25-225 | 170 | 95 |
| Formulations | Proline | 0-250 | 0 | 100 | 0-220 | 0 | 85 |
| Calcium | Calcium | 15-130 | 100 | 60 | 15-195 | 110 | 70 |
| Glutamate | Glutamate | 30-300 | 230 | 140 | 25-320 | 240 | 145 |
| Formulations | Proline | 0-250 | 0 | 70 | 0-220 | 0 | 60 |

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the disclosure (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted.

Recitation of ranges of values herein are merely intended to serve as a shorthand method of referring individually to each separate value falling within the range and each endpoint, unless otherwise indicated herein, and each separate value and endpoint is incorporated into the specification as if it were individually recited herein.

All methods described herein can be performed in any suitable order unless otherwise indicated herein or otherwise clearly contradicted by context. The use of any and all examples, or exemplary language (e.g., “such as”) provided herein, is intended merely to better illuminate the disclosure and does not pose a limitation on the scope of the disclosure unless otherwise claimed. No language in the specification should be construed as indicating any non-claimed element as essential to the practice of the disclosure.

Preferred embodiments of this disclosure are described herein, including the best mode known to the inventors for carrying out the disclosure. Variations of those preferred embodiments may become apparent to those of ordinary skill in the art upon reading the foregoing description. The inventors expect skilled artisans to employ such variations as appropriate, and the inventors intend for the disclosure to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

What is claimed:

1. An aqueous composition comprising (a) an anti-TSLP antibody at a concentration greater than about 140 mg/mL, (b) a surfactant, and (c) at least one basic amino acid or a salt thereof, wherein the composition comprises about 10 mM to about 200 mM basic amino acid or a salt thereof.

2. The aqueous composition of claim 1, wherein the basic amino acid is arginine.

3. The aqueous composition of claim 2, comprising an organic salt of arginine.

4. The aqueous composition of claim 3, wherein the arginine salt is arginine acetate, arginine aspartate, arginine glutamate, arginine glycolate, arginine lactate, arginine methanesulfonate, arginine propionate, or a combination thereof.

5. The aqueous composition of claim 1, wherein the basic amino acid is histidine.

6. The aqueous composition of claim 5, comprising an organic salt of histidine.

7. The aqueous composition of claim 6, wherein the histidine salt is histidine acetate, histidine aspartate, histidine glutamate, histidine glycolate, histidine lactate, histidine methanesulfonate, histidine propionate, or a combination thereof.

8. The aqueous composition of claim 1, wherein the basic amino acid is lysine.

9. The aqueous composition of claim 8, comprising an organic salt of lysine.

10. The aqueous composition of claim 9, wherein the lysine salt is lysine acetate, lysine aspartate, lysine glutamate, lysine glycolate, lysine lactate, lysine methanesulfonate, lysine propionate, or a combination thereof.

11. An aqueous composition comprising (a) an anti-TSLP antibody at a concentration greater than about 140 mg/mL, (b) a surfactant, and (c) at least one calcium salt or magnesium salt, wherein the composition comprises about 15 mM to about 150 mM calcium salt or magnesium salt.

12. The aqueous composition of claim 12, wherein the calcium salt or magnesium salt comprises a counterion lacking chloride.

13. The aqueous composition of claim 12, wherein the counterion is acetate, aspartate, glutamate, glycolate, lactate, methanesulfonate, propionate, or a combination thereof.

14. The aqueous composition of claim 13, wherein the calcium salt is calcium acetate, calcium aspartate, calcium glutamate, calcium glycolate, calcium lactate, calcium methanesulfonate, calcium propionate, or a combination thereof.

15. The aqueous composition of claim 13, wherein the magnesium salt is magnesium acetate, magnesium aspartate, magnesium glutamate, magnesium glycolate, magnesium lactate, magnesium methanesulfonate, magnesium propionate, or a combination thereof.

16. The aqueous composition of any one of the preceding claims, further comprising N-acetyl arginine (NAR), N-acetyl lysine, methionine, glycine, proline, sodium acetate, tris acetate, a histidine salt, or a calcium salt, optionally, in an amount of about 50 mM to about 250 mM

17. The aqueous composition of claim 16, comprising an arginine salt and NAR or methionine or a combination thereof.

18. The aqueous composition of claim 17, wherein the arginine salt is arginine glutamate.

19. The aqueous composition of claim 18, comprising 25-190 mM arginine base and 25-200 mM glutamic acid.

20. The aqueous composition of claim 18 or 19, comprising 140 mM arginine base and 150 mM glutamic acid.

21. The aqueous composition of any one of claims 18 to 20, comprising proline from 0 to 250 mM.

22. The aqueous composition of claim 21, comprising 80 mM aginine base, 85 mM glutamic acid and 100 mM L-Proline

23. The aqueous composition of any one of claims 18 to 22, comprising 0.01% (w/v) Polysorbate 80.

24. The composition of claim 23, comprising 140 mM arginine base, 150 mM glutamic acid, 0.01% (w/v) Polysorbate 80.

25. The composition of claim 23, comprising 80 mM arginine base, 85 mM glutamic acid, 100 mM L-Proline, 0.01% (w/v) Polysorbate 80.

26. The aqueous composition of claim 18, comprising 10-125 mM arginine base and 25-225 mM glutamic acid.

27. The aqueous composition of claim 18 or 26, comprising 95 mM arginine base and 170 mM glutamic acid.

28. The aqueous composition of any one of claims 26 to 27, comprising proline from 0 to 220 mM.

29. The aqueous composition of claim 28, comprising 50 mM arginine base, 95 mM glutamic acid and 85 mM L-Proline.

30. The aqueous composition of any one of claims 26 to 29, comprising 0.01% (w/v) polysorbate 80.

31. The aqueous composition of claim 30, comprising 95 mM arginine base, 170 mM glutamic acid, 0.01% (w/v) polysorbate 80.

32. The aqueous composition of claim 30, comprising 50 mM arginine base, 95 mM glutamic acid and 85 mM L-Proline, 0.01% (w/v) polysorbate 80.

33. The aqueous composition of any one of the preceding claims, wherein the anti-TSLP antibody comprises:
(A) a light chain variable domain comprising: (i) a light chain CDR1 sequence comprising the amino acid sequence set forth in SEQ ID NO:3; (ii) a light chain CDR2 sequence comprising the amino acid sequence set forth in SEQ ID NO:4; and (iii) a light chain CDR3 sequence comprising the amino acid sequence set forth in SEQ ID NO:5; and
(B) a heavy chain variable domain comprising: (i) a heavy chain CDR1 sequence comprising the amino acid sequence set forth in SEQ ID NO:6; (ii) a heavy chain CDR2 sequence comprising the amino acid sequence set forth in SEQ ID NO:7, and (iii) a heavy chain CDR3 sequence comprising the amino acid sequence set forth in SEQ ID NO:8.

34. The aqueous composition of claim 33, wherein the anti-TSLP antibody comprises:
(A) a light chain variable domain selected from the group consisting of:
i. a sequence of amino acids at least 80% identical to SEQ ID NO:12;
ii. a sequence of amino acids encoded by a polynucleotide sequence that is at least 80% identical to SEQ ID NO:11; or
iii. a sequence of amino acids encoded by a polynucleotide that hybridizes under moderately stringent conditions to the complement of a polynucleotide consisting of SEQ ID NO:11; or
(B) a heavy chain variable domain selected from the group consisting of:
i. a sequence of amino acids that is at least 80% identical to SEQ ID NO:10;
ii. a sequence of amino acids encoded by a polynucleotide sequence that is at least 80% identical to SEQ ID NO:9; or
iii. a sequence of amino acids encoded by a polynucleotide that hybridizes under moderately stringent conditions to the complement of a polynucleotide consisting of SEQ ID NO:9; or
(C) a light chain variable domain of (A) and a heavy chain variable domain of (B).

35. The aqueous composition of claim 17, comprising a calcium salt and NAR or methionine or a combination thereof.

36. The aqueous composition of claim 35, wherein the calcium salt is calcium glutamate.

37. The aqueous composition of claim 36, comprising 15-130 mM calcium and 30-300 mM glutamate.

38. The aqueous composition of claim 36 or 37, comprising 100 mM calcium and 230 mM glutamate.

39. The aqueous composition of any one of claims 35 to 38, comprising proline from 0 to 250 mM.

40. The aqueous composition of claim 39, comprising 60 mM calcium, 140 mM glutamate and 70 mM L-Proline

41. The aqueous composition of claim 35, comprising 15-195 mM calcium and 25-320 mM glutamate.

42. The aqueous composition of claim 35 or 41, comprising 110 mM calcium and 240 mM glutamate.

43. The aqueous composition of any one of claims 35 or 41 to 42, comprising proline from 0 to 220 mM.

44. The aqueous composition of claim 43, comprising 70 mM calcium, 145 mM glutamate and 60 mM L-Proline.

45. The aqueous composition of any one of claims 35 to 44, comprising 0.01% (w/v) polysorbate 80.

46. The aqueous composition of claim 45, comprising 100 mM calcium, 230 mM glutamate, and 0.01% (w/v) polysorbate 80.

47. The aqueous composition of claim 45, comprising 60 mM calcium, 140 mM glutamate, 70 mM L-Proline, 0.01% (w/v) polysorbate 80.

48. The aqueous composition of claim 45, comprising 110 mM calcium, 240 mM glutamate, 0.01% (w/v) polysorbate 80.

49. The aqueous composition of claim 45, comprising 70 mM calcium, 145 mM glutamate, 60 mM L-Proline, 0.01% (w/v) polysorbate 80.

50. The aqueous composition of any one of claims 35 to 49, wherein the anti-TSLP antibody comprises:
(A) a light chain variable domain comprising: (i) a light chain CDR1 sequence comprising the amino acid sequence set forth in SEQ ID NO:3; (ii) a light chain CDR2 sequence comprising the amino acid sequence set forth in SEQ ID NO:4; and (iii) a light chain CDR3 sequence comprising the amino acid sequence set forth in SEQ ID NO:5; and
(B) a heavy chain variable domain comprising: (i) a heavy chain CDR1 sequence comprising the amino acid sequence set forth in SEQ ID NO:6; (ii) a heavy chain CDR2 sequence comprising the amino acid sequence set forth in SEQ ID NO:7, and (iii) a heavy chain CDR3 sequence comprising the amino acid sequence set forth in SEQ ID NO:8.

51. The aqueous composition of claim 50, wherein the anti-TSLP antibody comprises:
(A) a light chain variable domain selected from the group consisting of:
iv. a sequence of amino acids at least 80% identical to SEQ ID NO:12;
v. a sequence of amino acids encoded by a polynucleotide sequence that is at least 80% identical to SEQ ID NO:11; or
vi. a sequence of amino acids encoded by a polynucleotide that hybridizes under moderately stringent conditions to the complement of a polynucleotide consisting of SEQ ID NO:11; or
(B) a heavy chain variable domain selected from the group consisting of:
iv. a sequence of amino acids that is at least 80% identical to SEQ ID NO:10;
v. a sequence of amino acids encoded by a polynucleotide sequence that is at least 80% identical to SEQ ID NO:9; or
vi. a sequence of amino acids encoded by a polynucleotide that hybridizes under moderately stringent conditions to the complement of a polynucleotide consisting of SEQ ID NO:9; or
(C) a light chain variable domain of (A) and a heavy chain variable domain of (B).

52. The aqueous composition of any one of the preceding claims, wherein the concentration of the anti-TSLP antibody is about 160 mg/mL to about 250 mg/mL.

53. The aqueous composition of claim 52, wherein the concentration of the anti-TSLP antibody is about 165 mg/mL to about 225 mg/mL.

54. The aqueous composition of claim 53, wherein the concentration of the anti-TSLP antibody is about 165 mg/mL to about 200 mg/mL.

55. The aqueous composition of claim 54, wherein the concentration of the anti-TSLP antibody is about 175 mg/mL to about 185 mg/mL, optionally, about 180 mg/mL.

56. The aqueous composition of claim 52, wherein the concentration of the anti-TSLP antibody is about 189 mg/mL to about 231 mg/mL.

57. The aqueous composition of claim 52, wherein the concentration of the anti-TSLP antibody is about 205 mg/mL to about 215 mg/mL, optionally, about 210 mg/mL.

58. The aqueous composition of any one of the preceding claims, having a pH of about 4.5 to about 6.75.

59. The aqueous composition of claim 58, having a pH of about 4.8 to about 6.0.

60. The aqueous composition of claim 59, comprising 25-190 mM arginine base and 25-200 mM glutamic acid.

61. The aqueous composition of claim 59 or 60, comprising 140 mM arginine base and 150 mM glutamic acid.

62. The aqueous composition of any one of claims 60 to 61, comprising proline from 0 to 250 mM.

63. The aqueous composition of claim 62, comprising 80 mM arginine base, 85 mM glutamic acid and 100 mM L-Proline.

64. The aqueous composition of any one of claims 60 to 63, comprising 0.01% (w/v) polysorbate 80, pH 5.4.

65. The aqeuous composition of claim 60, comprising 140 mM arginine base, 150 mM glutamic acid, 0.01% (w/v) polysorbate 80, pH 5.4.

66. The aqueous composition of claim 60, comprising 80 mM arginine base, 85 mM glutamic acid, 100 mM L-Proline, 0.01% (w/v) polysorbate 80, pH 5.4.

67. The aqueous composition of claim 59, comprising 10-125 mM arginine base and 25-225 mM glutamic acid.

68. The aqueous composition of claim 67, comprising 95 mM arginine base and 170 mM glutamic acid.

69. The aqueous composition of any one of claim 67 or 68, comprising proline from 0 to 220 mM.

70. The aqueous composition of claim 69, comprising 50 mM arginine base, 95 mM glutamic acid and 85 mM L-Proline.

71. The aqueous composition of any one of claims 67 to 70, comprising 0.01% (w/v) polysorbate 80, pH 5.4.

72. The aqueous composition of claim 71, comprising 95 mM arginine base, 170 mM glutamic acid, 0.01% (w/v) polysorbate 80, at pH 5.4.

73. The aqueous composition of claim 71, comprising 50 mM arginine base, 95 mM glutamic acid, 85 mM L-Proline, 0.01% (w/v) polysorbate 80, at pH 5.4.

74. The aqueous composition of claim 59 comprising 15-130 mM calcium and 30-300 mM glutamate.

75. The aqueous composition of claim 74 comprising 100 mM calcium and 230 mM glutamate.

76. The aqueous composition of any one of claim 74 or 75 comprising proline from 0 to 250 mM.

77. The aqueous composition of claim 76 comprising 60 mM calcium, 140 mM glutamate and 70 mM L-Proline.

78. The aqueous composition of claim 59 comprising 15-195 mM calcium and 25-320 mM glutamate.

79. The aqueous composition of claim 78 comprising 110 mM calcium and 240 mM glutamate.

80. The aqueous composition of any one of claim 78 or 79 comprising proline from 0 to 220 mM.

81. The aqueous composition of claim 80, comprising 70 mM calcium, 145 mM glutamate and 60 mM L-Proline.

82. The aqueous composition of any one of claims 78 to 81, comprising 0.01% (w/v) polysorbate 80, pH 5.0.

83. The aqueous composition of claim 82, comprising 100 mM calcium, 230 mM glutamate, 0.01% (w/v) polysorbate 80, at pH 5.0.

84. The aqueous composition of claim 82, comprising 60 mM calcium, 140 mM glutamate, 70 mM L-Proline, 0.01% (w/v) polysorbate 80, at pH 5.0.

85. The aqueous composition of claim 82, comprising 110 mM calcium, 240 mM glutamate, 0.01% (w/v) polysorbate 80, at pH 5.0.

86. The aqueous composition of claim 82, comprising 70 mM calcium, 145 mM glutamate, 60 mM L-Proline, 0.01% (w/v) polysorbate 80, at pH 5.0.

87. The aqueous composition of any one of the preceding claims, wherein the viscosity of the aqueous composition is less than 100 cP at 23° C., 1000 s⁻¹.

88. The aqueous composition of claim 87, wherein the viscosity of the aqueous composition is less than 75 cP at 23° C., 1000 s⁻¹.

89. The aqueous composition of claim 88, wherein the viscosity of the aqueous composition is less than 60 cP at 23° C., 1000 s⁻¹.

90. The aqueous composition of claim 89, wherein the viscosity of the aqueous composition is less than 50 cP at 23° C., 1000 s⁻¹.

91. The aqueous composition of any one of the preceding claims, wherein the surfactant is amphipathic and/or nonionic.

92. The composition of claim 91, wherein the surfactant is a polysorbate.

93. The composition of claim 92, wherein the surfactant is polysorbate 20 or polysorbate 80 or a mixture thereof.

94. The composition of any one of the preceding claims, comprising a surfactant at a concentration less than or about 0.005% (w/v) to about 0.015% (w/v).

95. The composition of claim 94, comprising about 0.010% (w/v)±0.0025% (w/v) surfactant.

96. The composition of claim 95, comprising about 0.005% (w/v), 0.010% (w/v), or 0.015% (w/v) surfactant

97. The composition of any one of the preceding claims, wherein the composition is isotonic or has an osmolality in a range of about 200 mOsm/kg to about 500 mOsm/kg, or about 225 mOsm/kg to about 400 mOsm/kg, or about 250 mOsm/kg to about 350 mOsm/kg.

98. The composition of any one of the preceding claims, wherein the composition is isotonic or has an osmolality greater than about 350 mOsm/kg.

99. The aqueous composition of any one of the preceding claims, wherein less than 5% of the antibody is degraded after storage at 2° C. to 8° C. for at least or about 12 months, as determined by Size Exclusion Chromatography (SEC).

100. The aqueous composition of claim 99, wherein less than 5% of the antibody is degraded after storage at 2° C. to 8° C. for about 20 months to about 26 months, as determined by Size Exclusion Chromatography (SEC).

101. The aqueous composition of claim 100, wherein less than 5% of the antibody is degraded after storage at 2° C. to 8° C. for about 30 to about 40 months, as determined by Size Exclusion Chromatography (SEC).

102. The aqueous composition of any one of the preceding claims, wherein less than 5% of the antibody is degraded after storage at a temperature greater than 20° C. for at least or about 2 weeks, as determined by Size Exclusion Chromatography (SEC), optionally, for at least or about 4 weeks or 8 weeks.

103. The aqueous composition of claim 102, wherein the temperature is greater than or about 25° C. or greater than or about 30° C. or greater than or about 40° C.

104. The aqueous composition of any one of the preceding claims, wherein the anti-TSLP antibody is an IgG2 antibody.

105. The aqueous composition of any one of claims 33 to 104, wherein the anti-TSLP antibody comprises a heavy chain comprising the amino acid sequence of SEQ ID NO: 13, a light chain comprising the amino acid sequence of SEQ ID NO: 14, or a heavy chain comprising the amino acid sequence of SEQ ID NO: 13 and a light chain comprising the amino acid sequence of SEQ ID NO: 14.

106. The aqueous composition of any one of claims 33 to 105, wherein the anti-TSLP antibody specifically binds to a TSLP polypeptide as set forth in amino acids 29-159 of SEQ ID NO: 2.

107. The aqueous composition of any one of claims 1 to 106, wherein both binding sites of the anti-TSLP antibody have identical binding to TSLP.

108. An article of manufacture comprising the aqueous composition of any one of the preceding claims, optionally, comprising about 1 mL to about 5 mL of the aqueous composition.

109. A pre-filled syringe comprising the aqueous composition of any one of the preceding claims, optionally, comprising about 1 mL to about 5 mL of the aqueous composition.

110. A vial comprising the aqueous composition of any one of the preceding claims, optionally, comprising about 1 mL to about 5 mL of the aqueous composition.

111. An autoinjector containing the aqueous composition of claim 107.

112. The auto-injector of claim 111 wherein the auto-injector is an Ypsomed YpsoMate®.

113. The auto-injector of claim 111 wherein the auto-injector is disclosed in WO 2018/226565, WO 2019/094138, WO 2019/178151, WO 20120/072577, WO2020/081479, WO 2020/081480, PCT/US20/70590, PCT/US20/70591, PCT/US20/53180, PCT/US20/53179, PCT/US20/53178, or PCT/US20/53176.

114. Use of the aqueous composition of any one of the preceding claims for treating an inflammatory disease.

115. The use of claim 114, wherein the inflammatory disease is selected from the group consisting of: asthma, atopic dermatitis, chronic obstructive pulmonary disease (COPD), nasal polyps, chronic spontaneous urticaria, eosinophilic esophagitis (EoE), Ig-driven disease (such as IgA nephropathy & lupus nephritis), eosinophilic gastritis, chronic sinusitis without nasal polyps and idiopathic pulmonary fibrosis (IPF).

116. The use of claim 115, wherein the inflammatory disease is atopic dermatitis.

117. A method for treating an inflammatory disease in a subject comprising administering to the subject a therapeutically effective amount of the aqueous composition of any one of the preceding claims.

118. The method of claim 117, wherein the inflammatory disease is selected from the group consisting of: asthma, atopic dermatitis, chronic obstructive pulmonary disease (COPD), nasal polyps, chronic spontaneous urticaria, eosinophilic esophagitis (EoE), Ig-driven disease (such as IgA nephropathy & lupus nephritis), eosinophilic gastritis, chronic sinusitis without nasal polyps and idiopathic pulmonary fibrosis (IPF).

119. The method of claim 118, wherein the inflammatory disease is atopic dermatitis.

120. The method of any one of claims 117 to 119, wherein the aqueous composition is administered to the subject by subcutaneous administration.

121. The method of any one of claims 117 to 120, wherein about 10 mL to about 20 mL of the aqueous composition is administered to the subject.

122. A method of making a stable, liquid antibody composition having a viscosity of less than about 100 cP and comprising (A) an anti-TSLP antibody at a concentration greater than about 140 mg/mL, (B) a surfactant, and (C) a basic amino acid or a salt thereof, a calcium salt, a magnesium salt, or a combination thereof, said method comprising: (i) combining the antibody with an aqueous solution comprising about 50 mM to about 150 mM basic amino acid or a salt thereof, a calcium salt, a magnesium salt, or a combination thereof and (ii) adding a surfactant to achieve a final concentration of about 0.01% (w/v)±0.005% (w/v) surfactant.


SEQUENCE LISTING (14 sequences)

SEQ ID NO: 1 - DNA, 743 bp, Homo Sapiens
  misc_feature: TSLP
  CDS (200)..(676)
gcagccagaaagctctggagcatcagggagactccaacttaaggcaacagcatgggtgaataagggcttcctgtggactggcaatgagaggcaaaacctggtgcttgagcactggcccctaaggcaggccttacagatctcttacactcgtggtgggaagagtttagtgtgaaactggggtggaattgggtgtccacgtatgttcccttttgccttactatatgttctgtcagtttctttcaggaaaatcttcatcttacaacttgtagggctggtgttaacttacgacttcactaactgtgactttgagaagattaaagcagcctatctcagtactatttctaaagacctgattacatatatgagtgggaccaaaagtaccgagttcaacaacaccgtctcttgtagcaatcggccacattgccttactgaaatccagagcctaaccttcaatcccaccgccggctgcgcgtcgctcgccaaagaaatgttcgccatgaaaactaaggctgccttagctatctggtgcccaggctattcggaaactcagataaatgctactcaggcaatgaagaagaggagaaaaaggaaagtcacaaccaataaatgtctggaacaagtgtcacaattacaaggattgtggcgtcgcttcaatcgacctttactgaaacaacagtaaaccatctttattatggtcatatttcacagcccaaaataaatcatctttattaagtaaaaaaaaa

SEQ ID NO: 2 - protein, 159 aa, Homo Sapiens
MFPFALLYVLSVSFRKIFILQLVGLVLTYDFTNCDFEKIKAAYLSTISKDLITYMSGTKSTEFNNTVSCSNRPHCLTEIQSLTFNPTAGCASLAKEMFAMKTKAALAIWCPGYSETQINATQAMKKRRKRKVTTNKCLEQVSQLQGLWRRFNRPLLKQQ

SEQ ID NO: 3 - protein, 11 aa, Homo Sapiens
  MISC_FEATURE: LCDR1
GGNNLGSKSVH

SEQ ID NO: 4 - protein, 7 aa, Homo Sapiens
  MISC_FEATURE: LCDR2
DDSDRPS

SEQ ID NO: 5 - protein, 11 aa, Homo Sapiens
  MISC_FEATURE: LCDR3
QVWDSSSDHVV

SEQ ID NO: 6 - protein, 5 aa, Homo Sapiens
  MISC_FEATURE: HCDR1
TYGMH

SEQ ID NO: 7 - protein, 17 aa, Homo Sapiens
  MISC_FEATURE: HCDR2
VIWYDGSNKHYADSVKG

SEQ ID NO: 8 - protein, 13 aa, Homo Sapiens
  MISC_FEATURE: HCDR3
APQWELVHEAFDI

SEQ ID NO: 9 - DNA, 366 bp, Homo Sapiens
  misc_feature: Heavy Chain VH
cagatgcagctggtggagtctgggggaggcgtggtccagcctgggaggtccctgagactctcctgtgcagcgtctggattcaccttcagaacctatggcatgcactgggtccgccaggctccaggcaagggactggagtgggtggcagttatatggtatgatggaagtaataaacactatgcagactccgtgaagggccgattcaccatcaccagagacaattccaagaacactctgaatctgcaaatgaacagcctgagagccgaggacacggctgtgtattactgtgcgagagcccctcagtgggagctagttcatgaagcttttgatatctggggccaagggacaatggtcaccgtctcttca

SEQ ID NO: 10 - protein, 122 aa, Homo Sapiens
  MISC_FEATURE: Heavy Chain VH
QMQLVESGGGVVQPGRSLRLSCAASGFTFRTYGMHWVRQAPGKGLEWVAVIWYDGSNKHYADSVKGRFTITRDNSKNTLNLQMNSLRAEDTAVYYCARAPQWELVHEAFDIWGQGTMVTVSS

SEQ ID NO: 11 - DNA, 325 bp, Homo Sapiens
  misc_feature: Light Chain VL
tcctatgtgctgactcagccaccctcggtgtcagtggccccaggacagacggccaggattacctgtgggggaaacaaccttggaagtaaaagtgtgcactggtaccagcagaagccaggccaggcccctgtgctggtcgtctatgatgatagcgaccggccctcatggatccctgagcgattctctggctccaactctgggaacacggccaccctgaccatcagcaggggcgaagccggggatgaggccgactattactgtcaggtgtgggatagtagtagtgatcatgtggtatttcggcggagggaccaagctgaccgtccta

SEQ ID NO: 12 - protein, 108 aa, Homo Sapiens
  MISC_FEATURE: Light Chain VL
SYVLTQPPSVSVAPGQTARITCGGNNLGSKSVHWYQQKPGQAPVLVVYDDSDRPSWIPERFSGSNSGNTATLTISRGEAGDEADYYCQVWDSSSDHVVFGGGTKLTVL

SEQ ID NO: 13 - protein, 5 aa, Artificial Sequence
  Synthetic peptide
DYWMH

SEQ ID NO: 14 - protein, 19 aa, Artificial Sequence
  Synthetic peptide
HIKSKTDAGTTDYAAPVKG